(19) United States
(12) Patent Application Publication
(10) Pub. No.: US 2014/0228242 A1
(43) Pub. Date: Aug. 14, 2014

(54) QUANTITATIVE GENOMICS OF THE RELAXATION RESPONSE

(71) Applicants: Herbert Benson, Brookline, MA (US); Gregory Fricchione, Harvard, MA (US); Jeffery A. Dusek, Minneapolis, MN (US); Towia Aron Libermann, Chesnut Hill, MA (US); Manoj Kumar Bhasin, Acton, MA (US)

(72) Inventors: Herbert Benson, Brookline, MA (US); Gregory Fricchione, Harvard, MA (US); Jeffery A. Dusek, Minneapolis, MN (US); Towia Aron Libermann, Chesnut Hill, MA (US); Manoj Kumar Bhasin, Acton, MA (US)

(73) Assignees: BETH ISRAEL DEACONESS MEDICAL CENTER, INC., Boston, MA (US); THE GENERAL HOSPITAL CORPORATION, Boston, MA (US)

(21) Appl. No.: 14/236,124

(22) PCT Filed: Aug. 3, 2012
(86) PCT No.: PCT/US12/49539

§ 371 Date: Apr. 10, 2014

Related U.S. Application Data
(60) Provisional application No. 61/514,770, filed on Aug. 3, 2011.

(51) Int. Cl. C12Q 1/68

(52) U.S. Cl. CPC C12Q 1/6876; C12Q 2600/156
USPC 506/9; 435/6.11; 506/17

(57) ABSTRACT

Disclosed herein are assays for and methods of monitoring the relaxation response in a subject by measuring the expression level of one or more genes that are modulated in a relaxation response. Primers, probes and kits for use in the assays and methods are also provided.

### RELATED APPLICATIONS

This application claims the benefit under 35 U.S.C. §119(e) of U.S. Provisional Application Ser. No. 61/514,770, filed Aug. 3, 2011, the contents of which are incorporated herein by reference in their entirety.

### GOVERNMENTAL SUPPORT

This invention was made with Government support under CDCDP00309, R01 DP000339 and H75/CCH123424, awarded by the Centers for Disease Control and Prevention, and under RO1 AT006464-01 awarded by the National Center for Complementary and Alternative Medicine (NCCAM), and under M01 RR01032, awarded by the National Center for Research Resources, of the National Institute of Health. The Government has certain rights in the invention.

### FIELD OF THE INVENTION

The present invention relates to the field of gene expression profiling and the therapeutic achievement of the relaxation response in a subject.

### BACKGROUND OF THE INVENTION

Mind body practices that elicit the relaxation response (RR), the counterpart of the stress response, have been used for thousands of years and according to national surveys are increasing in use for general well-being but also for specific disease states (NHIS 2002, 2007). Despite these observations and the well-established clinical effects of RR-eliciting practices (Astin et al., J Am Board Fam Pract. 2003; 16:131-147; Esch T, Med Sci Monit. 2003; 9:RA23-34), the mechanisms underlying the RR have not been identified. Previously a distinctive gene expression profile was identified in long-term RR practitioners compared with novices either before or after their 8-week RR training (Dusek et al., PLoS One 2008; 3(7):e2576). Similarly, genomic profiling of neutrophil transcripts identified distinctive gene expression profiles in Asian Qigong practitioners (Li et al., The Journal of Alternative and Complementary Medicine 2005; (11): 29-39). This disclosure follows up on these findings to analyze temporal gene expression changes associated with one session of relaxation response elicitation among various subjects.

### SUMMARY OF THE INVENTION

One aspect of the invention relates to an assay for the relaxation response in a subject comprising measuring the expression levels of one or more genes shown in Tables 2-8, and combinations thereof, in a biological sample of the subject prior to and subsequent to relaxation practice by the subject, and calculating the difference in the measured expression levels of the one or more genes, wherein an increase in gene expression subsequent to relaxation practice of one or more of the genes shown in Tables 2-4, and/or a decrease in gene expression of one or more of the genes shown in Tables 5-8, indicates the relaxation response in the subject.

Another aspect of the invention relates to a method of monitoring the relaxation response in a subject, comprising measuring the expression levels of one or more genes shown in Tables 2-8, and combinations thereof, in a biological sample of the subject prior to and subsequent to relaxation practice by the subject, and calculating the difference in the measured expression levels of the one or more genes, herein an increase in gene expression subsequent to relaxation practice of one or more of the genes shown in Tables 2-4 and/or a decrease in gene expression of one or more of the genes shown in Tables 5-8, indicates the relaxation response in the subject.

Another aspect of the invention relates to a method of generating a relaxation gene expression profile of a subject, comprising measuring the expression levels of one or more genes shown in Tables 2-8, and combinations thereof, in a biological sample of the subject prior to and subsequent to relaxation practice by the subject, calculating the difference in the measured expression levels of the one or more genes, and recording the respective calculated differences in the measured expression levels of the one or more gene, to thereby generate the relaxation gene expression profile of the subject.

In one embodiment of the methods, assays and compositions described herein, the one or more genes are NFX1, PTPN11, SNRPD2, SRC, HSPA5, PLCG1, YBX1, SOS1, SNRPB, MAPK14, PIK3R1, RHOA, TP53, LCK, MYC, MAPK8, PIK3CA, MAPK1, IKBKG, TRAF6, MAPK14, FYN, HSPA8, SHC1, POLR2H, MAPK31, JUN, U2AF2, RELA, ENSGOOOOO234745, DHX9, TP53, TRAF6, YWHAZ, FUS, PTK2B, HSPA8, JUN, JAK1, MYC, STX1A, POLR2A, INS, SSR4, PRKCA, PKM2, PDHA1, PRKACA, CBL, UGDH, FGFR1, RPL7, SRC, GAPDH, ATP5B, AHCY, ATP5C1, GNAI2, GRB2, GNB2, or combinations thereof.

In one embodiment of the methods, assays, systems and computer readable medium described herein, the one or more genes are NFX1, PTPN11, SNRPD2, SRC, HSPA5, PLCG1, YBX1, SOS1, SNRPB, MAPK14, DHX9, TP53, TRAF6, YWHAZ, FUS, PTK2B, HSPA8, JUN, JAK1, MYC, or combinations thereof, and a decrease in gene expression of one or more of the genes indicates the relaxation response in the subject.

In one embodiment of the methods and compositions described herein, the one or more genes are PIK3R1, RHOA, TP53, LCK, MYC, MAPK8, PIK3CA, MAPK1, IKBKG, TRAF6, MAPK14, FYN, HSPA8, SHC1, POLR2H, MAPK31, JUN, U2AF2, RELA, ENSGOOOOO234745, or combinations thereof, and a decrease in gene expression of one or more of the genes indicates the relaxation response in the subject.

In one embodiment of the methods and compositions described herein, the one or more genes are STX1A, POLR2A, INS, SSR4, PRKCA, PKM2, PDHA1, PRKACA, CBL, UGDH, FGFR1, RPL7, SRC, GAPDH, ATP5B, AHCY, ATP5C1, GNAI2, GRB2, GNB2, or combinations thereof, and an increase in gene expression of one or more of the genes indicates the relaxation response in the subject.

One aspect of the invention relates to an assay for the relaxation response in a subject comprising a) measuring the expression level of one or more relaxation responsive genes in a biological sample of the subject prior to and subsequent to relaxation practice by the subject, and

b) calculating any difference in the measured expression levels of the one or more relaxation responsive genes. A significant increase in gene expression subsequent to relaxation practice of one or more of the relaxation responsive genes that are relaxation response induced genes, and/or a decrease in gene expression subsequent to relaxation practice of one or more of the relaxation responsive genes that are relaxation response inhibited genes, indicates the relaxation response in the subject.

Another aspect of the invention relates to a method of monitoring the relaxation response in a subject, comprising a) measuring the expression level of one or more relaxation responsive genes in a biological sample of the subject prior to and subsequent to relaxation practice by the subject, and b) calculating any difference in the measured expression levels of the one or more genes. A significant increase in gene expression subsequent to relaxation practice of one or more of the relaxation responsive genes that are relaxation response induced genes, and/or a decrease in gene expression subsequent to relaxation practice of one or more of the relaxation responsive genes that are relaxation response inhibited genes, indicates the relaxation response in the subject.

Another aspect of the invention relates to a method of generating a relaxation gene expression profile of a subject, comprising a) measuring the expression levels of one or more relaxation responsive genes in a biological sample of the subject prior to and subsequent to relaxation practice by the subject, b) calculating any difference in the measured expression levels of the one or more genes, and c) recording the respective calculated differences in the measured expression levels of the one or more genes, to thereby generate the relaxation gene expression profile of the subject.

Another aspect of the invention relates to a method of determining effectiveness of relaxation practice by a subject, comprising a) generating a relaxation gene expression profile of the subject in response to relaxation practice by the subject, and b) comparing the relaxation gene expression profile generated to a standard relaxation gene expression profile indicative of a complete relaxation response. The similarity of the relaxation gene expression profile generated for the subject to the standard gene expression profile indicates effectiveness of the relaxation practice by the subject.

Another aspect of the invention relates to a method for treating a subject diagnosed with, or at risk for a disease or disorder, comprising a) assaying for the relaxation response in the subject by the methods described herein, and b) prescribing relaxation response therapy, in lieu of, or in combination with, drug therapy appropriate for treatment of the disease or disorder, for the subject if identified as exhibiting the relaxation response, or prescribing drug therapy appropriate for treatment of the disease or disorder for the subject if identified as not exhibiting the relaxation response.

Another aspect of the invention relates to a method for determining if relaxation practice will be of clinical benefit to a subject diagnosed with, or at risk for a disease or disorder, comprising, assaying for the relaxation response in the subject by the methods described herein. An indication of the relaxation response in the subject indicates that relaxation practice will be of clinical benefit to the subject, and a lack of an indication of the relaxation response in the subject indicates that relaxation practice will not be of clinical benefit to the subject.

Another aspect of the invention relates to a microarray for high throughput detection of gene expression levels in a biological sample comprising probes for one or more relaxation-responsive genes.

Another aspect of the invention relates to a system for analyzing a biological sample of a subject comprising a determination module configured to receive a biological sample and to determine gene expression information, wherein the gene expression information comprises levels of expression of one or more genes described herein modulated in the relaxation response, a storage device configured to store the information from the determination module, a comparison module adapted to compare the information stored on the storage device with reference data, and to provide a comparison result, wherein the comparison result is a relaxation gene expression profile of the subject, and a display module for displaying a content based in part on the comparison result for the user, wherein the content is a signal indicative of the genes modulated from baseline in the biological sample, wherein the content is indicative of the relaxation gene expression profile of the subject.

In one embodiment of the system described herein, the reference data is the baseline gene expression profile of the subject.

Another aspect of the invention relates to a system for analyzing a biological sample comprising a determination module configured to receive a biological sample and to determine gene expression information, wherein the gene expression information comprises, genes modulated from baseline in a subject as determined by the method described herein, a storage device configured to store the information from the determination module, a comparison module adapted to compare the information stored on the storage device with reference data, and to provide a comparison result, wherein the comparison result is indicative of the extent of a relaxation response in the subject, and a display module for displaying a content based in part on the comparison result for the user, wherein the content is a signal indicative of the extent of the relaxation response in the subject.

In one embodiment of the system described herein, the reference data is the relaxation gene expression profile of an accomplished practitioner of relaxation practice.

Another aspect of the invention relates to a computer readable medium having computer readable instructions recorded thereon to define software modules including a comparison module and a display module for implementing a method on a computer, said method comprising comparing with the comparison module the data stored on a storage device with reference data to provide a comparison result, wherein the comparison result is the relaxation gene expression profile of a subject, and displaying a content based in part on the comparison result for the user, wherein the content is a signal indicative of the relaxation gene expression profile of the subject.

In one embodiment of the computer readable medium described herein, the reference data is the baseline gene expression profile of the subject.

Another aspect of the invention relates to a computer readable medium having computer readable instructions recorded thereon to define software modules including a comparison module and a display module for implementing a method on a computer, said method comprising comparing with the comparison module the data stored on a storage device with reference data to provide a comparison result, wherein the comparison result is the extent of the relaxation response of a subject, and displaying a content based in part on the comparison result for the user, wherein the content is a signal indicative of the extent of the relaxation response of the subject.

In one embodiment of the computer readable medium described herein, the reference data is the relaxation gene expression profile of an accomplished practitioner of relaxation practice.

In one embodiment of the methods, assays, systems, compositions, and computer readable medium described herein, the one or more genes are selected from the group consisting of the genes shown in Table 15, or a subset thereof, the genes shown in Table 10, or a subset thereof, the genes shown in Table 11, or a subset thereof, the genes shown in Table 12, or a subset thereof, the genes shown in Table 13, or a subset thereof, the genes shown in Table 14 or a subset thereof, and combinations thereof.

In one embodiment of the methods, assays, systems, compositions, and computer readable medium described herein the one or more genes comprise NF-κB, RELA, MAPK14, MAPK, JNK, TNF, INS, ATP5C1, and ATP5B.

In one embodiment of the methods, assays, systems, compositions, and computer readable medium described herein the one or more genes comprise MAPK, JNK, NF-κB, IKBKB, TP53, MYC, and TNF.

In one embodiment of the methods, assays, systems, compositions, and computer readable medium described herein the one or more genes comprise MAPK, NJK, NF-κB, MAP3K and/or MYC.

In one embodiment of the methods, assays, systems, compositions, and computer readable medium described herein the one or more genes comprise the one or more genes comprise ATPASE, INS and/or INSR, MAPK14, JUN and/or JNK, NF-κB, MAPK, IKBKG and/or IKBKB, and MAPK.

In one embodiment of the methods, assays, systems, compositions, and computer readable medium described herein the one or more genes comprise ATP5C1, ATP5B, INS, PRKACA, MAPK14, JUN, RELA and/or NF-κB, MAPK8, IKBKG, MAP3K1, HSPA5, TP53, CCNA1C, CYC1, and RUVBL1.

In one embodiment of the methods, assays, systems, compositions, and computer readable medium described herein the relaxation practice is at least about 20 minutes in duration.

In one embodiment of the methods, assays, systems, compositions, and computer readable medium described herein the biological sample is whole blood.

In one embodiment of the methods, assays, systems, compositions, and computer readable medium described herein the biological sample is peripheral blood mononuclear cells.

In one embodiment of the methods, assays, systems, compositions, and computer readable medium described herein the measuring prior to the relaxation practice is performed within 2 minutes of relaxation practice by the subject.

In one embodiment of the methods, assays, systems, compositions, and computer readable medium described herein the measuring subsequent to relaxation practice is performed within 1 minute of completion of the relaxation practice by the subject.

In one embodiment of the methods, assays, systems, compositions, and computer readable medium described herein the measuring subsequent to relaxation practice is performed within 15 minutes post completion of the relaxation practice by the subject.

In one embodiment of the methods, assays, systems, compositions, and computer readable medium described herein the measuring subsequent to relaxation practice is performed at more than one time point.

In one embodiment of the methods, assays, systems, compositions, and computer readable medium described herein measuring the gene expression levels is performed by quantitative PCR, microarray analysis, luminex gene platform analysis, or next generation sequencing analysis.

In one embodiment of the methods, assays, systems, compositions, and computer readable medium described herein the subject is diagnosed with, or at risk for, a disease or disorder caused or exacerbated by stress, selected from the group consisting of hypertension, anxiety, depression, infertility, insomnia, menopausal symptoms, premenstrual symptoms, pains, phobias, nausea, post-traumatic stress disorder, obesity, impotency related to stress or anxiety, tinnitus or sensation of sounds, postoperative swelling, allergic skin reactions, bronchial asthma, congestive heart failure, constipation, cough, diabetes mellitus, drowsiness, duodenal ulcers, fatigue and dizziness, herpes simplex, irritable bowel syndrome and inflammatory bowel disease, rheumatoid arthritis, and multiple sclerosis.

In one embodiment of the methods, assays, systems, compositions, and computer readable medium described herein the relaxation practice is selected from the group consisting of Qigong, yoga, meditation, repetitive prayer, tai chi, breathing exercises, progressive muscle relaxation, biofeedback, hypnosis, autogenic training and guided imagery.

### DEFINITIONS

The term “differential expression” or “differentially expressed” refers to increased or upregulated gene expression or decreased or downregulated gene expression as detected by the absence, presence, or at least statistically significant difference in the amount of transcribed messenger RNA or translated protein in a sample as compared to an appropriate control (e.g., a baseline level of expression or a negative control subject).

The term “binding fragment member of a specific binding pair” refers to an entity that specifically binds a polypeptide or modification thereof, with significant affinity for experimental detection of that polypeptide in the bound form. Detection is typically via the detection of a label present on the binding fragment member. One example is an antibody or antigen binding fragment thereof, which binds a specific antigen.

The term “antibody” means an immunoglobulin that binds to a specific antigen. The term “antibody” refers to an immunoglobulin protein that is capable of binding an antigen present on an indicated target protein/polypeptide. Antigen binding fragments of an antibody (e.g. F(ab′)2, Fab′, Fab, Fv) are capable of binding the antigen or antigenic fragment of interest. Antibodies can be IgG, IgM, IgA, IgD, and IgE antibodies. Antigen binding fragments of antibodies are also used in the present invention. The term includes polyclonal, monoclonal, monovalent, humanized, heteroconjugate, antibody compositions with polyepitopic specificity, chimeric, bispecific antibodies.

The term “array” or “microarray” means an ordered arrangement of at least two probes on a substrate. Typically at least one of the probes is a control or standard and at least one of the probes is a diagnostic probe for determination of gene expression level. The arrangement of probes on the substrate assures that the size and signal intensity of each labeled complex formed between a probe and a sample polynucleotide or polypeptide is individually distinguishable (e.g., from about two to about 40,000 probes).

The term “relaxation practice” and “relaxation response practice” are used interchangeably herein to refer to the performance of a physical and/or mental routine that promotes relaxation of the mind and body of the practitioner. Examples of such routines include Qigong, yoga, meditation, repetitive prayer, tai chi, breathing exercises, progressive muscle relaxation, biofeedback and guided imagery. Other forms of relaxation practice are known or can be devised by the skilled practitioner.

A “relaxation inducing event”, as the term is used herein, refers to an experimentally controlled event to which a test subject is subjected. In one example, the relaxation inducing event is listening to a relaxation recording. In one embodiment, the relaxation inducing even is a relaxation practice as described herein.

The term “relaxation gene expression profile” or “relaxation profile” used interchangeably herein, refers to the determined gene expression pattern of a given set of genes in a subject as modulated by performance of relaxation practice or experience of a relaxation inducing event. The profile is generated by comparing the gene expression of a given set of examined genes the subject prior to experiencing the relaxation practice/inducing event to the gene expression of those examined genes of the subject subsequent to the subject experiencing the relaxation practice/inducing event. One or more genes known or suspected of being modulated in a relaxation response (e.g., the genes identified herein) are typically in the given set of examined genes. The profile can be viewed as a specific, personalized data set for a given subject at the time period in which it was generated. The profiles generated for a subject may change over time with respect to external factors in the life of the subject, such as social and psychological stresses and also experience and skill acquired with relaxation practice.

To “compare” levels of gene expression (e.g., the levels determined in relaxation gene expression profiles) means to detect gene expression levels in two samples and to calculate so as to determine whether the levels are equal or if one or the other is greater. One of the sample can be that of a standardized control. A comparison can be done between quantified levels, allowing statistical comparison and calculation between the two values to determine a statistically significant difference in measured expression levels. A significant change in gene expression detected between the samples, refers to an increase or decrease, that is at least about 1.2 fold. In one embodiment, the change is at least about 1.3, 1.4, 1.5, 1.6, 1.7, 1.8, or 1.9 fold. In one embodiment, the change is at least about 2.0 fold. The comparison and/or calculation is typically performed by a non-human machine.

Alternatively, a comparison can be done in the absence of quantification, for example using qualitative methods of detection such as visual assessment by a human (e.g, as compared to a standardized control in the samples).

The “relaxation response” (RR) is defined in the art as a mind-body intervention that offsets the physiological effects caused by stress (Wallace et al., Am J. Physiol. 1971; 221:795-799; Benson et al., Psychiatry. 1974; 37:37-46). A complete RR is characterized by the occurrence of a number of physiological responses, such as decreased oxygen consumption (Benson et al., J Human Stress. 1975; 1:37-44; Kesterson et al., Am J. Physiol. 1989; 256:R632-638; Warrenburg et al., J Behav Med. 1980; 3:73-93), decreased carbon dioxide elimination, reduced blood pressure, heart and respiration rate (Wallace et al., Am J. Physiol. 1971; 221:795-799; Benson et al., Psychiatry. 1974; 37:37-46; Beary et al., Psychosom Med. 1974; 36:115-120), prominent low frequency heart rate oscillations (Peng et al., Int J. Cardiol. 2004; 95:19-27) and alterations in cortical and subcortical brain regions (Lazar et al., Neuroreport. 2000; 11:1581-1585; Jacobs et al., Biofeedback Self Regul. 1996; 21:121-129). A complete relaxation response is generally a response that is achieved by an accomplished practioner of relaxation practice.

As the term is used herein, “treating” and “treatment” refers to an action which results in an improvement in the disease or disorder, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease.

The term “effective relaxation response” refers to at least partial success of a practioner at accomplishing the goal of relaxation from the relaxation practice in which they take part. This is evidence by the induction of gene expression profile that indicates at least partial gene modulation as is experienced by the accomplished relaxation practitioner under similar circumstances.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIG. 1A-FIG. 1C is a collection of schematics showing top focus gene hubs identified from significantly enriched upregulated pathways. FIG. 1A) is a schematic showing top 20 focus gene hubs identified in upregulated progressive patterns. FIG. 1B) The grey scale represent the Bottleneck ranks from 1 to 20. The Bottleneck rank is a significance level with smaller rank indicating increasing confidence. FIG. 1C) Top 20 upregulated genes short focus hubs.

FIG. 2A-FIG. 2B is a collection of schematics showing top focus gene hubs identified from significantly enriched downregulated pathways. FIG. 2A) Progressive RR pattern top 20 genes. FIG. 2B) Integrated network with Long-, Short and Progressive RR effects top 20 genes. The grey color scale represent the Bottleneck ranks from 1 to 20. The Bottleneck rank is a significance level with smaller rank indicating increasing confidence. FIG. 2C) Long RR pattern top 20 genes. FIG. 2D) Short RR pattern top 20 genes. FIG. 2E) RR pattern genes.

FIG. 3 is a schematic showing various embodiments of the invention. System 10, Determination module 40, Storage Device 30, Comparison module 80, Display module 110, Content 140, Computer readable medium 200, Decision 300, Program stop 400.

FIG. 4 is a schematic showing various embodiments of the invention. System 10, Determination module 40, Storage Device 30, Comparison module 80, Display module 110, Content 140, Computer readable medium 200, Decision 300, Program stop 400.

FIG. 5 is a schematic view of the temporal relaxation response study design and analysis plans. The transcriptome profiling was performed on peripheral blood mononuclear cells (PBMCs) collected immediately prior to (T0), immediately after (T1) and 15 minutes after (T2) listening to a 20-minute Education CD by the Novices (N1) or a 20-minute RR CD by the Short term practitioners (N2) and the Long term practitioners (M). The global transcriptome of PBMCs was profiled using HT_U133A arrays containing >22,000 transcripts. The transcriptome data were analyzed using high-level bioinformatics algorithms to identify differentially expressed transcripts, significantly affected pathways and systems biology networks that are related to RR elicitation. The expression patterns were generated from differentially expressed genes using Self-Organizing Maps (SOM) analysis. The results of the GSEA from all comparisons were classified to temporal patterns (e.g. Progressive, Long) by developing a R-language script.

FIG. 6A-FIG. 6B shows the results of individual gene-based differential expression analysis. FIG. 6A) Differentially expressed genes identified by 3 across-group comparisons (N1 vs. N2, N1 vs. M, and N2 vs. M) at T0, T1 and T2. Venn diagrams depict the overlap of genes identified by these 3 comparisons at each time point. FIG. 6B) Differentially expressed genes identified by 3 within-group comparisons at different time points (T0 vs. T1, T0 vs. T2 and T1 vs. T2). Venn diagrams depict the overlap of genes identified by the 3 comparisons within each group.

FIG. 7 is a box plot of data from experimental results which shows the temporal genomic expression patterns during one session of RR elicitation. Genes that were differentially expressed either across or within groups comparisons at different time points were used as the seed set of genes for Self-Organizing Map (SOM) analysis. These differentially expressed genes were partitioned to 18 separate maps according to Pearson correlation coefficient based distance metrics. Selected biologically interesting SOM maps were manually clustered into 4 biologically relevant categories based on the gene expression of N1, N2 and M groups at the 3 time points in one session of RR elicitation: Long-term Downregulation; Long-term Upregulation; Progressive Upregulation; and Progressive Downregulation. One representative pattern for each of these 4 biologically relevant categories is shown in the figure. The figure displays the box plot of the gene expression with X-axis representing time points and groups, and Y-axis representing scaled gene expression data from −1 to +1.

FIG. 8A-FIG. 8B shows significantly enriched pathways with progressive patterns identified using gene set enrichment analysis FIG. 8A) Upregulated Pathways, FIG. 8 B) Downregulated Pathways. The solid dots indicate significantly affected pathways (False Discovery Rate <25%) identified from across group comparisons (N1 vs. N2, N1 vs. M and N2 vs. M) at a particular time point (T0, T1 and T2). The asterisks represent significance and directionality of enrichment (P value <0.09*, P value <0.05**, P value <0.01***) identified from within group comparisons at different time points (T0 vs. T1, T0 vs. T2, T1 vs. T2). “+” indicates up-regulated and “*” indicates down-regulated enrichment of pathways respectively. Selected genes from representative pathways are shown in panels on the right side. Pathways with progressive patterns were enriched (up- or down-regulated) in N2 and M groups with greater significance of enrichments in M group. Furthermore, increasing enrichment over time within one session of RR elicitation was observed in M group.

FIG. 9A-FIG. 9B shows significantly enriched pathways with long-term patterns identified using gene set enrichment analysis. FIG. 9A) Upregulated Pathways, FIG. 9B) Downregulated Pathways. The solid dots indicate significantly affected pathways (False Discovery Rate <25%) identified from across group comparisons (N1 vs. N2, N1 vs. M and N2 vs. M) at a particular time point (T0, T1 and T2). The asterisks represent significance and directionality of enrichment (P value <0.09*, P value <0.05**, P value <0.01***) identified from within group comparisons at different time points (T0 vs. T1, T0 vs. T2, T1 vs. T2). “+” indicates up-regulated and “*” indicates down-regulated enrichment of pathways respectively. Selected genes from representative pathways are shown in panels on the right side. Pathways with long-term patterns were enriched (up- or down-regulated) only in M group. Furthermore, increasing enrichment over time within one session of RR elicitation was observed in M group.

FIG. 10A-FIG. 10C show schematics of interactive network and top focus gene hubs identified from significantly affected pathways. The figure represents the top focus genes. FIG. 10A) Progressive upregulated Pathways, FIG. 10B) Progressive downregulated Pathways, and FIG. 10C) Integrated network of Long-term and Progressive affected pathways. The top focus hubs were identified from complex interactive networks generated from pathways with progressive and long-term patterns. The focus gene hubs were identified using the bottleneck algorithm for identification of the most interactive molecules with tree like topological structure. The bottleneck algorithm ranks genes on the basis of significance level with smaller rank indicating increasing confidence. The greycolor scale represents the bottleneck ranks from 1 to 20.

FIG. 11 is schematic that shows a schematic view of the study design and analysis plans for the Hypertension and IBD/IBS relaxation response study.

### DETAILED DESCRIPTION OF THE INVENTION

Aspects of the present invention are made possible by the finding that a specific gene expression profile is elicited in an accomplished practitioner of relaxation practice (e.g, a subject with years of experience at one or more relaxation practices) when the subject experiences a relaxation inducing event in a clinical test setting. Gene expression profiles generated from such accomplished relaxation practitioners are reported herein. These profiles reflect the specific genes modulated, the direction of modulation and the extent of the modulation of expression. These profiles are representative of a complete relaxation response. Such profiles can be used to monitor a given test subject for effective relaxation practice. Individual genes that are upregulated and individual genes that are down-regulated in the relaxation response of the experienced practitioner are reported herein. By monitoring a change in the expression levels of one or more of the genes identified herein in a subject, and comparing that change to that typically exhibited by an accomplished relaxation practitioner, the subject can be analyzed for their ability to achieve a relaxation response and the quality of the relaxation response that is achieved.

The expression level of specific genes identified herein is altered following elicitation of a relaxation response in a subject. These genes are referred to herein as “relaxation responsive genes”. Genes identified herein as exhibiting an increase in expression resulting from the relaxation response are referred to herein as “relaxation response induced genes”. Genes identified herein as exhibiting a decrease in expression resulting from the relaxation response are referred to herein as “relaxation response inhibited genes”.

One aspect of the invention relates to a method of generating a relaxation gene expression profile of a subject. The method comprises measuring the expression levels of one or more genes identified herein, in a biological sample of the subject prior to and subsequent to relaxation practice by the subject. The difference in the measured expression levels of the one or more genes is calculated. The respective values for the calculated differences in the measured expression levels of the one or more genes are recorded. The recorded information generated for a given subject is the relaxation gene expression profile of that subject. The relaxation gene expression profile of the subject can be compared to an appropriate positive control relaxation gene expression profile to determine the level of achievement (none, partial or complete) of the relaxation response of the subject.

A succession of different relaxation gene expression profiles can be generated over a period of time for a given subject. One such time period can be a time period of instruction and practice of a particular form of relaxation practice (e.g. prior to beginning instruction, and following instruction and practice, etc.). In this way the relaxation gene expression profile can be used to determine whether the subject is achieving the desired goal of an effective relaxation response.

### Genes to Analyze for Expression Modulation

By generating a gene expression profile, and using it to compare to gene expression profiles of appropriate control subjects, one can diagnose aspects of the relaxation response which the subject is experiencing or not experiencing from their relaxation practice. By the methods described herein, one can quantitate and track upregulation and/or downregulation of specific genes in a subject upon the performance of a relaxation inducing event, as determined in a clinical setting. Methods of determining upregulation or downregulation of a gene or a set of genes are known in the art, examples of which are provided herein.

Although hundreds of genes were determined modulated in a relaxation response, considerable information can be gained by monitoring the expression levels of one or more subsets of these genes. Particularly useful subsets are identified herein.

In one embodiment, the profile generated by the methods described herein comprises the expression level of one or more genes identified herein. In one embodiment, the profile comprises the expression level of one or more of the genes recited in Tables 2-8, and 10-15. In one embodiment, the profile comprises the expression level of one or more of the genes recited in Table 2, 3, 4, 5, 6, 7, 8, 10, 11, 12, 13, 14 or 15. In one embodiment, the profile comprises the expression level of the genes recited in one or more of the Table 2, 3, 4, 5, 6, 7, 8, 10, 11, 12, 13, 14 or 15. In one embodiment, the profile comprises the expression level of genes recited in one or more of the Table 2, 3, 4, 5, 6, 7, 8, 10, 11, 12, 13, 14 or 15, with the exception of one or more genes (e.g., 1, 2, 3, 4, . . . 15 genes) listed in the respective table. In one embodiment, the profile comprises the expression level of a combination of one or more of the genes recited in one or more of Table 2-8, and 10-15. Any combination of genes in the Tables recited herein can be used to produce a relaxation gene expression profile. For example, the expression level of one or more genes from Table 2, 3, 4, 5, 6, 7, 8, 10, 11, 12, 13, 14 or 15 can be combined with the expression level of one or more genes from any other table or combination of tables. In one embodiment, a plurality (e.g., 1, 2, 3, 4, . . . 20) of specific genes recited herein is excluded from the profile or analysis made in a method of the invention.

In one embodiment, the expression level of one or more of the following groups of genes are assessed in the methods described herein:

NF-κB, RELA, MAPK14, MAPK, JNK, TNF, INS, ATP5C1, and ATP5B.

MAPK, JNK, NF-κB, IKBKB, TP53, MYC, and TNF.

MAPK, NJK, NF-κB, MAP3K and/or MYC.

ATPASE, INS and/or INSR, MAPK14, JUN and/or JNK, NF-κB, MAPK, IKBKG and/or IKBKB, and MAPK.

ATP5C1, ATP5B, INS, PRKACA, MAPK14, JUN, RELA and/or NF-κB, MAPK8, IKBKG, MAP3K1, HSPA5, TP53, CCNA1C, CYC1, and RUVBL1.

In one embodiment, a combination of two or more of the above, discussed groups are genes are assessed.

Monitoring the modulation of genes identified as progressive in the experiments described below is of particular use in determining whether or not a subject is progressing in skill with respect to relaxation practice, as the modulation of these genes is expected to progressively increase as the skill of the practitioner increases. The increase can be in the number of actual genes identified as modulated, and/or in the level of gene expression for each gene that is modulated.

Analysis of modulation of the genes described herein can be quantitative or qualitative or combinations thereof. As such, the profile generated by the methods described herein can be quantitative or qualitative, or a combination thereof.

Various genes identified herein as modulated in the relaxation response are particularly relevant to certain diseases or disorders (e.g., insulin receptor is relevant to diabetes). As such, one embodiment of the methods described herein is the performance on a subject diagnosed with, suspected of having or at risk for such a disease or disorder, and includes the analysis of the expression modulation of genes identified herein that are related to or directly involved in the disease or disorder. By way of non-limiting example, ATP5C1 is related to or directly involved in Alzheimer, dementia and Huntington disease. HIST1H2BC is related to or directly involved in diseases or disorders of DNA repair, replication, chromosomal stability, and also to telomere maintenance.

Another aspect of the invention relates to a method of determining effectiveness of relaxation practice by a subject. The method comprises generating a relaxation gene expression profile of the subject in response to relaxation practice by the subject, and comparing the relaxation gene expression profile generated to a standard relaxation gene expression profile indicative of a complete relaxation response. The similarity of the relaxation gene expression profile generated for the subject to the standard gene expression profile indicates effectiveness of the relaxation practice by the subject.

An “effective relaxation response” is indicated by one or more genes in the relaxation expression profile of a subject exhibiting modulation (partial or complete) similar to that of the accomplished practitioner under the same or similar circumstances. Exemplary gene modulation of an accomplished practitioner is reported herein. An effective relaxation response encompasses a continuum ranging from a partial to a complete response and is indicated by a relaxation gene expression profile that is similarly partially or completely modulated. The determination of a partial relaxation response in a subject is useful to indicate the beginnings of success, and also can be used to guide instruction and practice toward a more complete relaxation response. Partial modulation can be complete or partial modulation of only a subset of the genes monitored, as compared to that of an appropriate positive control subject.

Another aspect of the present invention relates to an assay for the relaxation response in a subject. The assay comprises measuring the expression levels of one or more genes identified herein (e.g., Tables 2-8) in a biological sample of the subject prior to (baseline) and subsequent to relaxation practice by the subject. The difference in the measured expression levels of the one or more genes is then calculated. An increase in gene expression of one or more of the relaxation response induced genes (e.g., genes shown in Tables 2-4) subsequent to relaxation practice, and/or a decrease in gene expression of one or more of the relaxation response inhibited genes (e.g., genes shown in Tables 5-8) subsequent to relaxation practice, indicates effective relaxation response in the subject. The extent of the relaxation response can be quantitated and will correlate with the extent of gene modulation, with respect to the number of genes, the combination of genes modulated, and also the level of each individual gene modulation (e.g., complete or incomplete, when incomplete the extent as compared to complete modulation). The assay serves as a method of monitoring the relaxation response in the subject.

A detected increase or decrease in gene expression level that is considered useful in the methods described herein is a reproducible, statistically significant modulation (increase or decrease). Modulation exhibited/detected may be complete or incomplete. In one embodiment the modulation of a specific gene is complete (e.g., 100% as compared to modulation generally experienced a positive control subject). In one embodiment the modulation is substantial (e.g., 95%, 90%, 85%, 80%, 75%, 70%, 65%, 60%, 55%, 50%, 45%, 40%, 35%, 30% as compared to modulation generally experienced a positive control subject). In one embodiment the modulation is significant (e.g., 25%, 20%, 15%, 10%). In one embodiment, the modulation is detectable and relevant (e.g., 5%-2% as compared to modulation generally experienced a positive control subject)).

The extent of analysis of the gene expression modulation in a subject can vary. Extensive and quantitative analysis as well as less extensive and qualitative analysis is envisioned. Whether specific genes are or are not modulated to any extent described herein, in the subject can be determined. In one embodiment, a positive or negative result for a given gene is provided indicated in the analysis (qualitative). In one embodiment, the extent of a positive result is provided (quantitative). Combinations of such output for various genes is also envisioned as determined useful by the skilled practitioner. The number of total genes that are analyzed for expression modulation that exhibit modulation can also be calculated/measured. In one embodiment, all genes (100%) analyzed exhibit some modulation indicative of a relaxation response. In one embodiment, few than 100% (e.g., 95%, 90%, 85%, 80%, 75%, 70%, 65%, 60%, 55%, 50%, 45%, 40%, 35%, 30%, 25%, 20%, 15%, 10%, 5%-2%, as compared to modulation generally experienced a positive control subject) exhibit modulation.

Measuring the level of expression of the gene(s) in the methods described herein is performed prior to the relaxation practice to provide a baseline, and also subsequent to the relaxation practice. Such measurements are made using biological samples obtained from the subject at the indicated times and treated so that the contents reflect the gene expression at the time the sample is taken.

Measurements taken prior to the relaxation practice can be made immediately prior to (e.g., between 1 and 2 minutes prior to) or relatively prior to (e.g., between 3-5, 5-15, 15-20, 20-30 minutes prior to) or longer before (e.g., from 0.5-1, 1-2, 2-3 hrs) the relaxation practice.

In one embodiment, the measuring subsequent to relaxation practice is performed within 1 minute of completion of the relaxation practice by the subject. In one embodiment, the measuring subsequent to relaxation practice is performed within 15 minutes of completion of the relaxation practice by the subject. In one embodiment, the measuring subsequent to relaxation practice is performed within 30 minutes of completion of the relaxation practice by the subject. Useful time period subsequent to relaxation practice can be determined by the skilled practitioner, and include, without limitation 10, 20, 30, 40, 50, 60, 90, 120 minutes following completion of the relaxation practice.

In one embodiment, the biological sample is obtained by use of an indwelling catheter to obtain a blood sample from the subject at the indicated times prior to and subsequent to the practice.

In one embodiment, the indwelling catheter is placed in the subject who then sits quietly for 15 minutes. At the end of these 15 minutes, blood is drawn in order to provide a baseline. Immediately after (within 2 min), the subject starts the relaxation practice (otherwise referred to herein as the relaxation response technique). Blood is drawn again with the same catheter after the relaxation practice.

The time of the relaxation practice experienced in the herein described methods can vary. In one embodiment, the relaxation practice is about 20 minutes. In one embodiment, the relaxation practice is about 10 minutes. In one embodiment, the relaxation practice is about 5 minutes. Longer period of relaxation practice may also be useful, for example 30, 35, 40, 45, 50, 55, and 60 minutes. Benefit may also be had from extended periods of relaxation practice, e.g., 1.5 hours, 2 hours, 2.5 hours, 3 hours, etc.

Expression levels of the gene(s) in the methods described herein are determined either quantitatively or qualitatively. Quantitative detection of a level is by determining the amount of an agent (protein or nucleic acid) in a sample. Quantitative detection is useful, for example, when comparing to a standard level obtained from an appropriate positive control subject. Qualitative detection of a level is by determining the amount of the agent in question and comparing it to the amount of another like agent (e.g., another mRNA when measuring an mRNA, or another protein when measuring a protein) in the same sample, typically measured by the same technique and under the same conditions.

An appropriate positive control subject is a subject who has several years of experience at relaxation practice, who practices regularly (e.g., every day). Such a subject is referred to herein as an accomplished relaxation practitioner or an accomplished practitioner of relaxation practice. A positive control gene expression profiles can also be generated from a survey of a plurality of such subjects. The positive control subject should be exposed to the same conditions as the test subject, for the same periods of time, and have biological samples taken at identical intervals. Biological sample processing of a positive control is also optimally identical to that of the test subject.

A biological sample useful for the methods described herein includes blood (e.g., whole blood), and other biological fluids which potentially contain gene expression nucleic acid products or protein products. Such biological fluids include, without limitation, semen, urine, blood, serum, saliva, cerebrospinal fluid, nipple aspirates, and supernatant from cell lysate. The use of isolates of a biological sample in the methods of the invention is also envisioned. As used herein, an “isolate” of a biological sample (e.g., an isolate of a tissue or biological fluid sample) refers to a material or composition (e.g., a biological material or composition) which has been separated, derived, extracted, purified or isolated from the sample and preferably is substantially free of undesirable compositions and/or impurities or contaminants associated with the biological sample. In one embodiment, an isolate of a biological fluid is comprised of one or more specific cell types present in the fluid (e.g., leukocytes, neutrophils, eosinophil, basophyls, lymphocytes (T-cell, B-cell, natural killer), monocytes, erythrocytes, myoblasts, macrophages, dendritic cells, and combinations thereof). In another embodiment, an isolate of blood is plasma. In one embodiment, the isolate is peripheral blood mononuclear cells isolated from whole blood of the subject.

Proper performance of relaxation practice has multiple health benefits. In addition to the general overall health benefit conferred to a subject from experiencing the RR, a number of diseases and disorders in a subject can be therapeutically treated. A disease or disorder for which experiencing a relaxation response may prove therapeutic to the sufferer includes, without limitation, diseases or disorders caused by or exacerbated by stress. Such diseases or disorders include, without limitation, hypertension, anxiety, depression, infertility, insomnia, menopausal symptoms, premenstrual symptoms, pains, phobias, nausea, post-traumatic stress disorder, obesity, impotency related to stress or anxiety, tinnitus or sensation of sounds, postoperative swelling, allergic skin reactions, bronchial asthma, congestive heart failure, constipation, cough, diabetes mellitus, drowsiness, duodenal ulcers, fatigue and dizziness, herpes simplex, irritable bowel syndrome and inflammatory bowel disease, rheumatoid arthritis, and multiple sclerosis (Samuelson et al., Journal of Alternative and Complementary Medicine 2010, 16(2): 1-6; Benson et al., Psychotherapy and Psychosomatics 1978, 30(3-4): 229-42; Benson et al., Journal of Chronic Diseases 27 (1974): 163-69; Benson et al., The Lancet (1974): 289-91; Peters et al., American Journal of Hypertension 2001, 14(6 Pt. 1): 546-52; Kagan et al., Menopause 13(5): 727-29; Irvin et al., Journal of Psychosomatic Obstetrics and Gynecology, 1996, 17: 202-7; Figueroa-Mosley et al., Journal of the National Comprehensive Cancer Network, 2007, 5(1): 44-50; Caudill et al., the Clinical Journal of Pain 7 (1991): 305-10). In addition, a relaxation response may also be therapeutic to a subject experiencing complications arising during pregnancy, or to a subject who has received an organ transplant. As such, a subject who is diagnosed with or suspected of having, or at risk for, one or more diseases or disorders that can benefit from a relaxation response is an appropriate subject for participation in the methods described herein.

Another aspect of the invention relates to a method for determining if relaxation practice is of clinical benefit to a subject diagnosed with or at risk for a disease or disorder that would benefit from successful relaxation practice. The subject is analyzed for the relaxation response by the method described herein. For instance, the subject can experience a single round of relaxation practice, as described herein, and their relaxation response assayed. Relaxation practice is expected to be of clinical benefit to a subject who is seen to exhibit at least a partial relaxation response.

Another aspect of the invention relates to a method for treating a subject diagnosed with, or at risk for a disease or disorder described herein. In the method, the subject is assayed for whether or not they would benefit from relaxation practice. For instance, the subject can experience a single round of relaxation practice, as described herein, and their relaxation response assayed. A subject who is seen to exhibit at least a partial relaxation response can be prescribed relaxation therapy. Such a prescription can be in lieu of drug therapy. This can be particularly beneficial to a subject who is exhibiting borderline signs of the disease or disorders (e.g., borderline hypertension). A subject who fails to exhibit any relaxation response could instead be prescribed drug therapy for the disease or disorder. In one embodiment, the subject who fails to exhibit any relaxation response could also be prescribed relaxation therapy in combination with drug therapy. That subject could be monitored over time for the ability to exhibit the relaxation response from the relaxation practice. Such a subject could then, for example, be prescribed lower and lower amounts of drug therapy if and when they begin to exhibit a relaxation response from the relaxation practice.

Another aspect of the invention relates to a method of teaching a relaxation practice. The method comprises providing instruction to a subject in a form of relaxation practice, and using the methods described herein on the subject in order to monitor the relaxation response in the subject following performance by the subject of the relaxation practice. The instruction is then adjusted accordingly in light of the information provided by the assay results.

Various mind-body approaches can be used to elicit the RR, and as such the methods described herein can be used with any such approaches as a form a relaxation practice. These include various forms of meditation, repetitive prayer, yoga, tai chi, breathing exercises, progressive muscle relaxation, biofeedback, guided imagery, hypnosis, autogenic training, and Qi Gong (Benson H. TINS. 1983; 6:281-284). One way that the relaxation response can be elicited is when individuals repeat a word, sound, phrase, prayer or focus on their breathing with a disregard of intrusive everyday thoughts (Benson et al., Psychiatry. 1974; 37:37-46). Typically the relaxation practice used in the methods described herein is standardized in the form of a relaxation inducing event experienced in a clinical setting.

Gene expression levels can be measured by measuring the amount of transcribed RNA from the expressed genes, or by measuring the amount of protein product produced from the transcribed RNA.

### Methods of Measuring Gene Expression via Nucleic Acids

Decreased or increased gene expression can be measured at the nucleic acid level (e.g., RNA level) using any of the methods well known in the art for the quantitation of polynucleotides, such as, for example, PCR (including, without limitation, RT-PCR and qPCR), RNase protection, luminex gene platform analysis, next generation sequencing analysis Northern blotting, microarray (Lee and Saeed. Methods Mol Biol. 2007; 353:265-300), macroarray, and other hybridization methods. The nucleic acids that are measured for expression according to the invention are typically in the form of mRNA or reverse transcribed mRNA. The genes may be amplified to produce the nucleic acid that is measured.

General methods for RNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a biological sample tumor can be isolated, for example, by cesium chloride density gradient centrifugation. Additionally, large numbers of biological samples can readily be processed using techniques well known to those of skill in the art, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to an intrinsic gene of the present invention, or any derivative DNA or RNA. Hybridization of an mRNA with the probe indicates that the intrinsic gene in question is being expressed.

In one embodiment, the mRNA is immobilized on a solid surface and contacted with a probe, for example by running the isolated mRNA on an agarose gel and transferring the mRNA from the gel to a membrane. In one embodiment, the probes are immobilized on a solid surface and the mRNA is contacted with the probes, for example, in an Agilent gene chip array.

Another method for determining the level of gene expression RNA product in a sample involves the process of nucleic acid amplification, for example, by RT-PCR (U.S. Pat. No. 4,683,202), ligase chain reaction (Barany, Proc. Natl. Acad. Sci. USA 88:189-93, 1991), self sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using commonly known techniques.

Gene expression can be assessed by quantitative RT-PCR. Numerous different PCR or QPCR protocols are known in the art and exemplified herein below and can be directly applied or adapted for use using the compositions described herein for the detection and/or quantification of the genes identified herein. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, SMARTCYCLER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (Biorad Laboratories, Hercules, Calif.) and MX4000® (Stratagene, La Jolla, Calif.).

Quantitative PCR (QPCR) (also referred as real-time PCR) is preferred under some circumstances because it provides not only a quantitative measurement, but also reduced time and contamination.

In one embodiment, arrays (e.g., microarrays) are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample.

One aspect of the invention relates to a collection of nucleic acid probes specific for one or more of the sets or subsets of genes identified herein. In one embodiment, the nucleic acid probes are immobilized to a solid substrate. In one embodiment, the solid substrate and nucleic acid probes comprise a microarray or gene chip.

Another aspect of the invention relates to a kit comprising the above-discussed collection of nucleic acid probes. In one embodiment, the kit further comprises instructions for use in the methods described herein. In one embodiment, the kit further comprises one or more reagents for use in performing the methods described herein.

### Methods of Measuring Gene Expression via the Protein Product

The expressed protein product of a gene can be used to measure gene expression. Quantitative detection of a protein can be performed for a given biological sample. In one embodiment, a protein binding fragment of a specific binding pair (e.g., an antibody or antigen binding fragment thereof) can be used to detect and measure the level of specific proteins in the sample. Techniques useful for quantitative or qualitative detection of specific proteins can be used in the methods described herein. Such methods include, without limitation, immunodetection methods (western blot analysis, ELIZA, immunofluorescence), mass spectrometry, etc. Protein activity can also be assayed to detect gene expression modulation.

### Probes and Kits for Gene Expression Profiling

Kits for gene profiling are also encompassed by the present invention. Such kits can contain probes or primers used to detect expression of the various combinations of genes described herein.

In one embodiment, the capture probes are immobilized on an array. By “array” is intended a solid support or a substrate with peptide or nucleic acid probes attached to the support or substrate. Arrays typically comprise a plurality of different capture probes that are coupled to a surface of a substrate in different, known locations. The arrays of the invention comprise a substrate having a plurality of capture probes that can specifically bind an intrinsic gene expression product. The number of capture probes on the substrate varies with the purpose for which the array is intended. The arrays may be low-density arrays or high-density arrays and may contain 4 or more, 8 or more, 12 or more, 16 or more, 32 or more addresses, but will minimally comprise capture probes for the 20 genes listed in Tables 2-8, or combinations thereof.

The oligonucleotide primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as discussed infra. The kit may further comprise reagents and instructions sufficient for the amplification of expression products from the genes.

In an embodiment, the kit comprises nucleic acid probes/primers that binds specifically with a nucleic acid (e.g., RNA) or a fragment of the nucleic acid of the gene(s) recited herein. The kit may further comprise means for performing PCR reactions. The kit may further comprise media and solution suitable for taking a sample and for extracting RNA from said biological sample (e.g., blood sample). The kit can further comprise additional components for carrying out the method of the invention, such as RNA extraction solutions, purification column and buffers and the like. The kit of the invention can further include any additional reagents, reporter molecules, buffers, excipients, containers and/or devices as required described herein or known in the art, to practice a method of the invention.

Another aspect of the invention relates to a set of probes for detecting the expression levels of the one or more genes described herein. In one embodiment the probes are within a composition. In one embodiment, the probes comprise (a) polynucleotides that specifically hybridize to two or more genes from the sets or subets described herein (e.g., Tables 2-8 and 10-15), or fragments thereof, or (b) polypeptide binding agents that specifically bind to two or more polypeptides selected from proteins encoded by genes from the sets or subsets described herein (e.g., Tables 2-8 and 10-15), or fragments thereof.

One aspect of the invention relates to a collection of nucleic acid probes specific for one or more of the sets or subsets of genes identified herein. In one emboidment, the nucleic acid probes are immobilized to a solid substrate. In one embodiment, the solid substrate and nucleic acid probes comprise a microarray or gene chip.

Another aspect of the invention relates to a kit comprising the above-discussed collection of nucleic acid probes. In one embodiment, the kit further comprises instructions for use in the methods described herein. In one emboidment, the kit further comprises one or more reagents for use in performing the methods described herein.

### Data Gathering and Storage

In order to facilitate ready access, e.g., for comparison, review, recovery, and/or modification, the expression profiles are typically recorded in a database. Most typically, the database is a relational database accessible by a computational device, although other formats, e.g., manually accessible indexed files of expression profiles as photographs, analogue or digital imaging readouts, spreadsheets, etc. can be used. Regardless of whether the expression patterns initially recorded are analog or digital in nature, the expression patterns, expression profiles (collective expression patterns), and molecular signatures (correlated expression patterns) are stored digitally and accessed via a database. The database can be compiled and maintained at a central facility, with access being available locally and/or remotely.

Embodiments of the invention also provide for systems (and computer readable medium for causing computer systems) to perform a method for determining a relaxation gene expression profile of a subject, and also for determining or the extent of similarity of a relaxation gene expression profile with that of an accomplished practitioner (indicative of the extent of the relaxation response in a subject based on gene expression levels).

### Automated Detection, Quantitation, and Comparison of Measurements

In the various assays and methods described herein, the detection and/or quantitation of the measurements can be performed by a non-human machine, such as a computer. In the various assays and methods described herein, the comparison of the various gene expression levels can be performed by a non-human computer to thereby produce the calculations and determine the differences in expression levels recited in the methods.

Some aspects of the invention relate to functional modules, which are defined by computer executable instructions recorded on computer readable media and which cause a computer to perform method steps described herein when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible media that can be accessed by a computer. Computer readable media includes volatile and nonvolatile, removable and non-removable tangible media implemented in any method or technology for storage of information such as computer readable instructions, data structures, program modules or other data. Computer readable media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

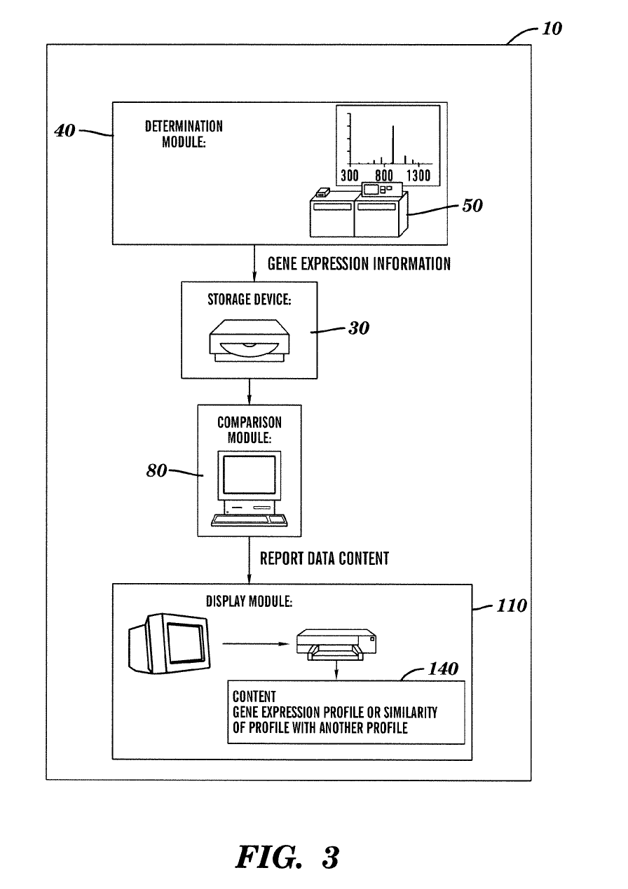

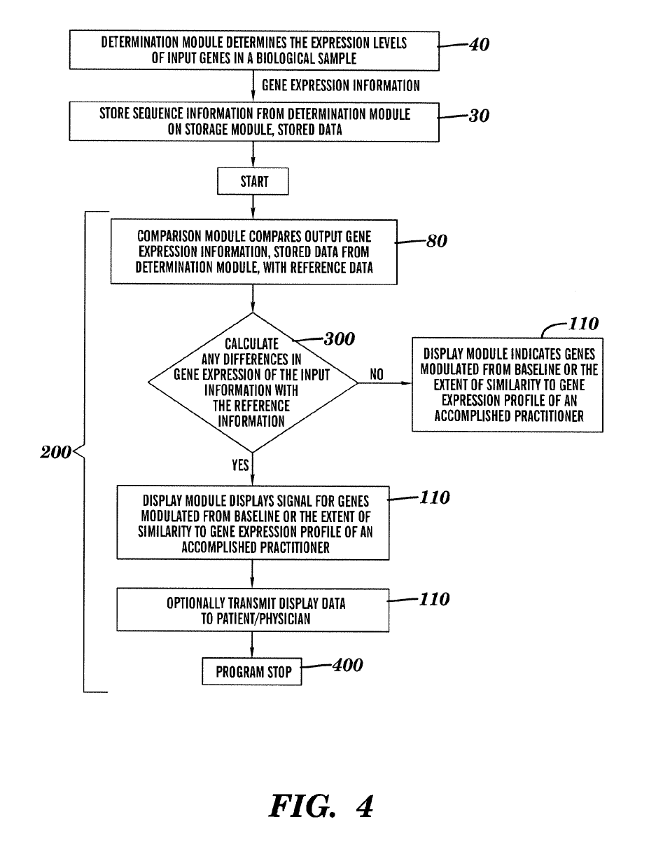

The following description refers to FIGS. 3 and 4. Computer-readable data generated and collected by the methods described herein embodied on one or more computer-readable media, or computer readable medium 200, may define instructions, for example, as part of one or more programs, that, as a result of being executed by a computer, instruct the computer to perform one or more of the functions described herein (e.g., in relation to system 10, or computer readable medium 200), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system 10, or computer readable medium 200 described herein, may be distributed across one or more of such components, and may be in transition there between.

The computer-readable media may be transportable such that the instructions stored thereon can be loaded onto any computer resource to implement the aspects of the present invention discussed herein. In addition, it should be appreciated that the instructions stored on the computer readable media, or the computer-readable medium 200, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The functional modules of certain embodiments of the invention include a determination module, a storage device, a comparison module and a display module. The functional modules can be executed on one, or multiple, computers, or by using one, or multiple, computer networks. The determination module 40 has computer executable instructions to provide sequence information in computer readable form. As used herein, “gene expression information” refers to the levels of expression of genes described herein, as detected by one or more methods described herein. The term “gene expression information” is intended to include the presence or absence of gene expression, as well as specific levels of detected expression as calculated by the methods described herein.

As an example, determination modules 40 for determining gene expression level information may include known systems for automated sequence analysis including but not limited to Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.:; ABI PRISM® 377 DNA Sequencer, ABI® 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics FluorImager™ 575, SI Fluorescent Scanners, and Molecular Dynamics FluorImager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England).

Alternative methods for determining gene expression levels i.e. determination modules 40, include systems for protein and DNA analysis. For example, mass spectrometry systems including Matrix Assisted Laser Desorption Ionization—Time of Flight (MALDI-TOF) systems and SELDI-TOF-MS ProteinChip array profiling systems; systems for analyzing gene expression data (see, for example, published U.S. Patent Application, Pub. No. U.S. 2003/0194711); systems for array based expression analysis: e.g., HT array systems and cartridge array systems such as GeneChip® AutoLoader, Complete GeneChip® Instrument System, GeneChip® Fluidics Station 450, GeneChip® Hybridization Oven 645, GeneChip® QC Toolbox Software Kit, GeneChip® Scanner 3000 7G plus Targeted Genotyping System, GeneChip® Scanner 3000 7G Whole-Genome Association System, GeneTitan™ Instrument, and GeneChip® Array Station (each available from Affymetrix, Santa Clara, Calif.); automated ELISA systems (e.g., DSX® or DS2® (available from Dynax, Chantilly, Va.) or the Triturus® (available from Grifols USA, Los Angeles, Calif.), The Mago® Plus (available from Diamedix Corporation, Miami, Fla.); Densitometers (e.g. X-Rite-508-Spectro Densitometer® (available from RP Imaging™, Tucson, Ariz.), The HYRYS™ 2 HIT densitometer (available from Sebia Electrophoresis, Norcross, Ga.); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

The gene expression level information determined in the determination module can be read by the storage device. As used herein the “storage device” 30 is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices 30 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon sequence information or expression level information. Such information may be provided in digital form that can be transmitted and read electronically, e.g., via the Internet, on diskette, via USB (universal serial bus) or via any other suitable mode of communication.

As used herein, “gene expression level information” refers to any nucleotide and/or amino acid expression level information, including but not limited to full-length nucleotide and/or amino acid sequences, partial nucleotide and/or amino acid sequences, or mutated sequences. Moreover, information “related to” the expression level information includes detection of the presence or absence of a sequence (e.g., presence or absence of an amino acid sequence, nucleotide sequence, or post translational modification), determination of the concentration of a sequence in the sample (e.g., amino acid sequence levels, or nucleotide (RNA or DNA) expression levels, or level of post translational modification), and the like.

As used herein, “stored” refers to a process for encoding information on the storage device 30. Those skilled in the art can readily adopt any of the presently known methods for recording information on known media to generate manufactures comprising the sequence information or expression level information.

A variety of software programs and formats can be used to store the sequence information or expression level information on the storage device. Any number of data processor structuring formats (e.g., text file or database) can be employed to obtain or create a medium having recorded thereon the sequence information or expression level information.

By providing gene expression level information in computer-readable form, one can use the sequence information or expression level information in readable form in the comparison module 80 to compare a specific sequence or expression profile with the reference data within the storage device 30. For example, search programs can be used to identify fragments or regions of the sequences that match a particular sequence (reference data, e.g., sequence information obtained from a control sample) or direct comparison of the determined expression level can be compared to the reference data expression level (e.g., information obtained from a control sample). The comparison made in computer-readable form provides a computer readable comparison result which can be processed by a variety of means. Content 140 based on the comparison result can be retrieved from the comparison module 80 to indicate a relaxation gene expression profile and the extent of a relaxation response of a subject.

In one embodiment the reference data stored in the storage device 30 to be read by the comparison module 80 is gene expression levels or sequence information data obtained from a control biological sample of the same type as the biological sample to be tested. Alternatively, the reference data are a database, e.g., an expression level profile (RNA, protein or peptide). In one embodiment the reference data are baseline gene expression levels or expression level profiles that are indicative of a relaxation gene expression profile and the extent of a relaxation response of a subject.

In one embodiment, the reference data are electronically or digitally recorded and annotated from databases including, but not limited to GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting information can be stored in a relational data base that may be employed to determine homologies between the reference data or genes or proteins within and among genomes.

The “comparison module” 80 can use a variety of available software programs and formats for the comparison operative to compare sequence information determined in the determination module 40 to reference data. In one embodiment, the comparison module 80 is configured to use pattern recognition techniques to compare sequence information from one or more entries to one or more reference data patterns. The comparison module 80 may be configured using existing commercially-available or freely-available software for comparing patterns, and may be optimized for particular data comparisons that are conducted. The comparison module 80 provides computer readable information related to the sequence information that can include, for example, detection of the presence or absence of a sequence (e.g., detection of a mutation or deletion (protein or DNA), information regarding distinct alleles, detection of post-translational modification, or omission or repetition of sequences); determination of the concentration of a sequence in the sample (e.g., amino acid sequence/protein expression levels, or nucleotide (RNA or DNA) expression levels, or levels of post-translational modification), or determination of a relaxation gene expression profile, or the extent of similarity of a relaxation gene expression profile with that of an accomplished practitioner (indicative of the extent of the relaxation response in a subject).

In one embodiment, the comparison module 80 permits the prediction of protein sequences from polynucleotide sequences, permits prediction of open reading frames (ORF), or permits prediction of homologous sequence information in comparison to reference data, i.e., homologous protein domains, homologous DNA or RNA sequences, or homologous exons and/or introns.

In one embodiment, the comparison module 80 uses sequence information alignment programs such as BLAST (Basic Local Alignment Seartch Tool) or FAST (using the Smith-Waternan algorithm) may be employed individually or in combination. These algorithms determine the alignment between similar regions of sequences and a percent identity between sequences. For example, alignment may be calculated by matching, bases-by-base or amino acid-by amino-acid.

The comparison module 80, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the comparison module 80 performs comparisons with mass-spectometry spectra, for example comparisons of peptide fragment sequence information can be carried out using spectra processed in MATLB with script called “Qcealign” (see for example WO2007/022248, herein incorporated by reference) and “Qpeaks” (Spectrum Square Associates, Ithaca, N.Y.), or Ciphergen Peaks 2.1™ software. The processed spectra can then be aligned using alignment algorithms that align sample data to the control data using minimum entropy algorithm by taking baseline corrected data (see for example WIPO Publication WO2007/022248, herein incorporated by reference). The comparison result can be further processed by calculating ratios. Protein expression profiles can be discerned.

In one embodiment, computational algorithms are used in the comparison module 80, such as expectation-maximization (EM), subtraction and PHASE are used in methods for statistical estimation of haplotypes (see, e.g., Clark, A. G. Mol Biol Evol 7:111-22 (1990); Stephens, M., Smith, N. J. & Donnelly, P. Am J Hum Genet 68:978-89 (2001); Templeton, A. R., Sing, C. F., Kessling, A. & Humphries, Genetics 120:1145-54 (1988)).

Various algorithms are available which are useful for comparing data and identifying the predictive gene signatures. For example, algorithms such as those identified in Xu et al., Physiol. Genomics 11:11-20 (2002). There are numerous software available for detection of SNPs and polymorphisms that can be used in the comparison module, including, but not limited to: HaploSNPer, a web-based program for detecting SNPs and alleles in user-specified input sequences from both diploid and polyploid species (available on the world-wide web at bioinformatics.nl/tools/haplosnper/; see also Tang et al., BMC Genetics 9:23 (2008)); Polybayes, a tool for SNP discovery in redundant DNA sequences (Marth, G T., et al., Nature Genetics 23(4):452-6 (1999); SSAHA-SNP, a polymorphism detection tool that uses the SSAHA alignment algorithm (available from Wellcome Trust Sanger Institute, Cambridge, United Kingdom, see also Ning Z., et al., Genome Research 11(10):1725-9 (2001)); Polyphred, A SNP discovery package built on phred, phrap, and consed tools (available on the world-wide web, see Nickerson, D A et al., Nucleic Acids Research 25(14):2745-51 (1997)); NovoSNP, a graphical Java-based program (PC/Mac/Linux) to identify SNPs and indels (available on the world-wide web, see Weckx, S. et al., Genome Research 15(3):436-442 (2005)); SNPdetector™, for automated identification of SNPs and mutations in fluorescence-based resequencing reads (available from Affymetrix, Santa Clara, Calif.), see also Zhang et al. PLoS Comput Biol (5):e53 (2005). SNPdetector runs on Unix/Linux platform and is available publicly; Affymetrix (Santa Clara, Calif.) has multiple data analysis software that can be used, for example Genotyping Console™ Software, GeneChip® Sequence Analysis Software (GSEQ), GeneChip® Targeted Genotyping Analysis Software (GTGS) and Expression Console™ Software.

In one embodiment, the comparison module 80 compares gene expression profiles (e.g., before and after relaxation practice, or a relaxation profile of a subject to an accomplished practitioner of relaxation practice). For example, detection of gene expression profiles can be determined using Affymetrix Microarray Suite software version 5.0 (MAS 5.0) (available from Affymetrix, Santa Clara, Calif.) to analyze the relative abundance of a gene or genes on the basis of the intensity of the signal from probe sets, and the MAS 5.0 data files can be transferred into a database and analyzed with Microsoft Excel and GeneSpring 6.0 software (available from Agilent Technologies, Santa Clara, Calif.). The detection algorithm of MAS 5.0 software can be used to obtain a comprehensive overview of how many transcripts are detected in given samples and alows a comparative analysis of 2 or more microarray data sets.

In one embodiment, the comparison module 80 compares protein expression profiles. Any available comparison software can be used, including but not limited to, the Ciphergen Express (CE) and Biomarker Patterns Software (BPS) package (available from Ciphergen Biosystems, Inc., Freemont, Calif.). Comparative analysis can be done with protein chip system software (e.g., The Proteinchip Suite (available from Bio-Rad Laboratories, Hercules, Calif.). Algorithms for identifying expression profiles can include the use of optimization algorithms such as the mean variance algorithm (e.g. JMP Genomics algorithm available from JMP Software Cary, N.C.).

In one embodiment of the invention, pattern comparison software is used to determine whether patterns of expression or mutations are indicative of a disease.

The comparison module 80 provides computer readable comparison result that can be processed in computer readable form by predefined criteria, or criteria defined by a user, to provide a content based in part on the comparison result that may be stored and output as requested by a user using a display module 110. The display module 110 enables display of a content based in part on the comparison result for the user, wherein the content 140 is a signal indicative of a relaxation gene expression profile, or the extent of similarity of a relaxation gene expression profile with that of an accomplished practitioner (indicative of the extent of the relaxation response in a subject).

Such signal, can be for example, a display of content 140 indicative of a relaxation gene expression profile, or the extent of similarity of a relaxation gene expression profile with that of an accomplished practitioner (indicative of the extent of the relaxation response in a subject), on a computer monitor, a printed page of content 140 indicating a relaxation gene expression profile, or the extent of similarity of a relaxation gene expression profile with that of an accomplished practitioner (indicative of the extent of the relaxation response in a subject) from a printer, or a light or sound indicative of a relaxation gene expression profile, or the extent of similarity of a relaxation gene expression profile with that of an accomplished practitioner (indicative of the extent of the relaxation response in a subject).

The content 140 based on the comparison result may include an expression profile of one or more proteins, or an expression profile of one or more genes. In one embodiment, the content 140 based on the comparison includes a sequence of a particular gene or protein and a determination of the presence of one or more mutations, or specific post-translational modification. In one embodiment, the content 140 based on the comparison result is merely a signal indicative of a relaxation gene expression profile, or the extent of similarity of a relaxation gene expression profile with that of an accomplished practitioner (indicative of the extent of the relaxation response in a subject).

In one embodiment of the invention, the content 140 based on the comparison result is displayed a on a computer monitor. In one embodiment of the invention, the content 140 based on the comparison result is displayed through printable media. In one embodiment of the invention, the content 140 based on the comparison result is displayed as an indicator light or sound. The display module 110 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content 140 based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to the sequence information, e.g., display of an indication of the presence or absence of mutation or deletion (DNA or protein); display of expression levels of an amino acid sequence (protein); display of nucleotide (RNA or DNA) expression levels; display of expression, SNP, or mutation profiles, or haplotypes, or display of information based thereon. In one embodiment, the sequence information of the reference sample data is also displayed.

In one embodiment, the display module 110 displays the comparison result and whether the comparison result is indicative of a relaxation response in a subject.

In one embodiment, the content 140 based on the comparison result that is displayed is a signal (e.g. positive or negative signal) indicative of a relaxation response in the subject, thus only a positive or negative indication may be displayed.

The present invention therefore provides for systems 10 (and computer readable medium 200 for causing computer systems) to perform methods for determining a relaxation gene expression profile, or the extent of similarity of a relaxation gene expression profile with that of an accomplished practitioner (indicative of the extent of the relaxation response in a subject), or whether the subject exhibits a relaxation response based on expression profiles.

System 10, and computer readable medium 200, are merely an illustrative embodiments of the invention for performing methods described herein, and is not intended to limit the scope of the invention. Variations of system 10, and computer readable medium 200, are possible and are intended to fall within the scope of the invention.

The modules of the system 10 or used in the computer readable medium 200, may assume numerous configurations. For example, function may be provided on a single machine or distributed over multiple machines.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.
1. An assay for the relaxation response in a subject comprising:
   a) measuring the expression level of one or more relaxation responsive genes in a biological sample of the subject prior to and subsequent to relaxation practice by the subject; and
   b) calculating any difference in the measured expression levels of the one or more relaxation responsive genes;
   wherein a significant increase in gene expression subsequent to relaxation practice of one or more of the relaxation responsive genes that are relaxation response induced genes, and/or a decrease in gene expression subsequent to relaxation practice of one or more of the relaxation responsive genes that are relaxation response inhibited genes, indicates the relaxation response in the subject.
2. A method of monitoring the relaxation response in a subject, comprising:
   a) measuring the expression level of one or more relaxation responsive genes in a biological sample of the subject prior to and subsequent to relaxation practice by the subject; and
   b) calculating any difference in the measured expression levels of the one or more genes;
   wherein a significant increase in gene expression subsequent to relaxation practice of one or more of the relaxation responsive genes that are relaxation response induced genes, and/or a decrease in gene expression subsequent to relaxation practice of one or more of the relaxation responsive genes that are relaxation response inhibited genes, indicates the relaxation response in the subject.
3. A method of generating a relaxation gene expression profile of a subject, comprising:
   a) measuring the expression levels of one or more relaxation responsive genes in a biological sample of the subject prior to and subsequent to relaxation practice by the subject;
   b) calculating any difference in the measured expression levels of the one or more genes; and
   c) recording the respective calculated differences in the measured expression levels of the one or more genes;
      to thereby generate the relaxation gene expression profile of the subject.
4. The method of any one of paragraphs 1-3, wherein the one or more genes are selected from the group consisting of the genes shown in Table 15, or a subset thereof, the genes shown in Table 10, or a subset thereof, the genes shown in Table 11, or a subset thereof, the genes shown in Table 12, or a subset thereof, the genes shown in Table 13, or a subset thereof, the genes shown in Table 14 or a subset thereof, and combinations thereof.
5. The method of any one of paragraphs 1-3, wherein the one or more genes comprise NF-κB, RELA, MAPK14, MAPK, JNK, TNF, INS, ATP5C1, and ATP5B.
6. The method of any one of paragraphs 1-3, wherein the one or more genes comprise MAPK, JNK, NF-κB, IKBKB, TP53, MYC, and TNF.
7. The method of any one of paragraphs 1-3, wherein the one or more genes comprise MAPK, NJK, NF-κB, MAP3K and/or MYC.
8. The method of any one of paragraphs 1-3, wherein the one or more genes comprise ATPASE, INS and/or INSR, MAPK14, JUN and/or JNK, NF-κB, MAPK, IKBKG and/or IKBKB, and MAPK.
9. The method of any one of paragraphs 1-3, wherein the one or more genes comprise ATP5C1, ATP5B, INS, PRKACA, MAPK14, JUN, RELA and/or NF-κB, MAPK8, IKBKG, MAP3K1, HSPA5, TP53, CCNA1C, CYC1, and RUVBL1.
10. The method of any one of paragraphs 1-9, wherein the relaxation practice is at least about 20 minutes in duration.
11. The method of any one of paragraphs 1-9, wherein the biological sample is whole blood.
12. The method of any one of paragraphs 1-9, wherein the biological sample is peripheral blood mononuclear cells.
13. The method of any one of paragraphs 1-12, wherein the measuring prior to the relaxation practice is performed within 2 minutes of relaxation practice by the subject.
14. The method of any one of paragraphs 1-13, wherein the measuring subsequent to relaxation practice is performed within 1 minute of completion of the relaxation practice by the subject.
15. The method of any one of paragraphs 1-13, wherein the measuring subsequent to relaxation practice is performed within 15 minutes post completion of the relaxation practice by the subject.
16. The method of any one of paragraphs 1-13, wherein the measuring subsequent to relaxation practice is performed at more than one time point.
17. The method of any one of paragraphs 1-16, wherein measuring the gene expression levels is performed by quantitative PCR, microarray analysis, luminex gene platform analysis, or next generation sequencing analysis.
18. A method of determining effectiveness of relaxation practice by a subject, comprising:
   a) generating a relaxation gene expression profile of the subject in response to relaxation practice by the subject; and
   b) comparing the relaxation gene expression profile generated to a standard relaxation gene expression profile indicative of a complete relaxation response;
      wherein the similarity of the relaxation gene expression profile generated for the subject to the standard gene expression profile indicates effectiveness of the relaxation practice by the subject.
19. The method of any one of paragraphs 1-18, wherein the subject is diagnosed with, or at risk for, a disease or disorder caused or exacerbated by stress, selected from the group consisting of hypertension, anxiety, depression, infertility, insomnia, menopausal symptoms, premenstrual symptoms, pains, phobias, nausea, post-traumatic stress disorder, obesity, impotency related to stress or anxiety, tinnitus or sensation of sounds, postoperative swelling, allergic skin reactions, bronchial asthma, congestive heart failure, constipation, cough, diabetes mellitus, drowsiness, duodenal ulcers, fatigue and dizziness, herpes simplex, irritable bowel syndrome and inflammatory bowel disease, rheumatoid arthritis, and multiple sclerosis.
20. The method of any one of paragraphs 1-19, wherein the relaxation practice is selected from the group consisting of Qigong, yoga, meditation, repetitive prayer, tai chi, breathing exercises, progressive muscle relaxation, biofeedback, hypnosis, autogenic training and guided imagery.
21. A method for treating a subject diagnosed with, or at risk for a disease or disorder, comprising:
   a) assaying for the relaxation response in the subject by the method of any one of paragraphs 1-20; and
   b) prescribing relaxation response therapy, in lieu of, or in combination with, drug therapy appropriate for treatment of the disease or disorder, for the subject if identified as exhibiting the relaxation response, or prescribing drug therapy appropriate for treatment of the disease or disorder for the subject if identified as not exhibiting the relaxation response.
22. A method for determining if relaxation practice will be of clinical benefit to a subject diagnosed with, or at risk for a disease or disorder, comprising, assaying for the relaxation response in the subject by the method of any one of paragraphs 1-20, wherein an indication of the relaxation response in the subject indicates that relaxation practice will be of clinical benefit to the subject, and a lack of an indication of the relaxation response in the subject indicates that relaxation practice will not be of clinical benefit to the subject.
23. The method of one of paragraphs 21 or 22, wherein the disease or disorder is caused or exacerbated by stress and is selected from the group consisting of hypertension, anxiety, depression, infertility, insomnia, menopausal symptoms, premenstrual symptoms, pains, phobias, nausea, post-traumatic stress disorder, obesity, impotency related to stress or anxiety, tinnitus or sensation of sounds, postoperative swelling, allergic skin reactions, bronchial asthma, congestive heart failure, constipation, cough, diabetes mellitus, drowsiness, duodenal ulcers, fatigue and dizziness, herpes simplex, irritable bowel syndrome and inflammatory bowel disease, rheumatoid arthritis, and multiple sclerosis.
24. A microarray for high throughput detection of gene expression levels in a biological sample comprising probes for one or more relaxation-responsive genes.
25. The microarray of paragraph 25, wherein the one or more relaxation-responsive genes are selected from the group consisting of the genes shown in Table 15, or a subset thereof, the genes shown in Table 10, or a subset thereof, the genes shown in Table 11, or a subset thereof, the genes shown in Table 12, or a subset thereof, the genes shown in Table 13, or a subset thereof, the genes shown in Table 14 or a subset thereof, and combinations thereof.
26. The microarray of paragraph 25, wherein the one or more genes comprise NF-κB, RELA, MAPK14, MAPK, JNK, TNF, INS, ATP5C1, and ATP5B.
27. The microarray of paragraph 25, wherein the one or more genes comprise MAPK, JNK, NF-κB, IKBKB, TP53, MYC, and TNF.
28. The microarray of paragraph 25, wherein the one or more genes comprise MAPK, NJK, NF-κB, MAP3K and/or MYC.
29. The microarray of paragraph 25, wherein the one or more genes comprise ATPASE, INS and/or INSR, MAPK14, JUN and/or JNK, NF-κB, MAPK, IKBKG and/or IKBKB, and MAPK.
30. The microarray of paragraph 25, wherein the one or more genes comprise ATP5C1, ATP5B, INS, PRKACA, MAPK14, JUN, RELA and/or NF-κB, MAPK8, IKBKG, MAP3K1, HSPA5, TP53, CCNA1C, CYC1, and RUVBL1.
31. A system for analyzing a biological sample of a subject comprising:
   a) a determination module configured to receive a biological sample and to determine gene expression information, wherein the gene expression information comprises:
      1) levels of expression of one or more relaxation responsive genes;
   b) a storage device configured to store the information from the determination module;
   c) a comparison module adapted to compare the information stored on the storage device with reference data, and to provide a comparison result, wherein the comparison result is a relaxation gene expression profile of the subject; and
   d) a display module for displaying a content based in part on the comparison result for the user, wherein
      1) the content is a signal indicative of the genes modulated from baseline in the biological sample, wherein the content is indicative of the relaxation gene expression profile of the subject.
32. The system of paragraph 31 wherein the reference data is the baseline gene expression profile of the subject.
33. A system for analyzing a biological sample comprising:
   a) a determination module configured to receive a biological sample and to determine gene expression information, wherein the gene expression information comprises:
      1) genes modulated from baseline in a subject as determined in paragraph 1 or paragraph 2
   b) a storage device configured to store the information from the determination module;
   c) a comparison module adapted to compare the information stored on the storage device with reference data, and to provide a comparison result, wherein the comparison result is indicative of the extent of a relaxation response in the subject; and
   d) a display module for displaying a content based in part on the comparison result for the user, wherein
      1) the content is a signal indicative of the extent of the relaxation response in the subject.
34. The system of paragraph 33, wherein the reference data is the relaxation gene expression profile of an accomplished practitioner of relaxation practice.
35. A computer readable medium having computer readable instructions recorded thereon to define software modules including a comparison module and a display module for implementing a method on a computer, said method comprising:
   a) comparing with the comparison module the data stored on a storage device with reference data to provide a comparison result, wherein the comparison result is the relaxation gene expression profile of a subject; and
   b) displaying a content based in part on the comparison result for the user, wherein the content is a signal indicative of the relaxation gene expression profile of the subject.
36. The computer readable medium of paragraph 35, wherein the reference data is the baseline gene expression profile of the subject.
37. A computer readable medium having computer readable instructions recorded thereon to define software modules including a comparison module and a display module for implementing a method on a computer, said method comprising:
   a) comparing with the comparison module the data stored on a storage device with reference data to provide a comparison result, wherein the comparison result is the extent of the relaxation response of a subject; and
   b) displaying a content based in part on the comparison result for the user, wherein the content is a signal indicative of the extent of the relaxation response of the subject.
38. The computer readable medium of paragraph 37, wherein the reference data is the relaxation gene expression profile of an accomplished practitioner of relaxation practice.

The invention is further illustrated by the following examples, which should not be construed as further limiting.

### EXAMPLES

### Example 1

### Methods

The study subjects were 26 healthy RR long-term practitioners (group M), and 26 healthy controls before (group N1) and after completing 8 weeks of RR training (group N2). During a laboratory session where M and N2 groups listened to a RR-eliciting CD and N1 group listened to a health education CD, blood samples were collected (i) immediately prior to, (ii) immediately after and (iii) 15 minutes after listening to the respective CDs. We assessed whole blood transcriptional profiles on the blood samples to compare the genomic changes at these three time points among M, N1 and N2 groups. The comparison was performed on each individual gene and also on groups of genes linked to biological pathways using Gene Set Enrichment Analysis (GSEA). We then performed system biology analysis to identify the RR signature and the key focus gene hubs that might be playing a critical role in RR. The expression pattern of selected genes (e.g. most modulated or focus gene hubs of RR) was further validated by quantitative polymerase chain reaction (QPCR).

### Results

Analysis of the individual time points identified a set of genes with significantly differential expressions at each of the three time points between long-term (M) as well as short-term (N2) practitioners and novices (N1). These genes are associated with a distinct set of pathways including blood pressure regulation, neurotransmitter regulation, and leukocyte migration. We also identified genes whose expressions are significantly different only between M and N1 groups. These genes are associated with cell cycle, cell adhesion, and insulin secretion pathways.

GSEA of M vs N1 and N2 vs N1 transcriptional profiles revealed a set of upregulated pathways with progressively higher levels for the former comparison (progressive pattern). These pathways are linked to energy metabolism (electron transport chain, integration of energy metabolism) and insulin secretion pathways. Similarly, GSEA identified downregulated pathways that had the progressive expression pattern and are linked to inflammatory processes (NF-κB, TNF R2, CCR5, IL-7, RELA). The analysis also identified several pathways that were significantly downregulated in M vs N1 and M vs N2 only (long term pattern). These pathways are linked to immune response (TCR signaling, Antigen Processing and Presentation, IL10 signaling), cell cycle (apoptotic pathways) as well as well as stress-related pathways (stress pathway, P38 MAPK). In upregulated long-term patterns, we identified overrepresentation of pathways linked to telomere maintenance and cardiac muscle contraction.

Further systems biology analysis using interactive network analysis of genes from significantly modulating pathways identified Insulin Receptor (INSR) and a mitochondrial ATP synthase (ATP5C1) as top upregulated focus hub genes. Similarity an interactive network of downregulated genes consists of many upstream and downstream targets of the NF-κB complex indicting its critical role in RR.

### Gene Set Enrichment Analysis of Enriched Biological Pathways

Gene Set Enrichment Analysis (GSEA) of M versus N1 and N2 versus N1 transcriptional profiles revealed a set of upregulated pathways with progressively higher levels for the former comparison (progressive pattern). These pathways are linked to energy metabolism (electron transport chain, integration of energy metabolism) and insulin secretion pathways (Table 1A). Similarly, GSEA analysis identified downregulated pathways that had the progressive expression pattern and are linked to inflammatory processes (NF-κB, TNF R2, CCR5, IL-7, RELA) and T cell signaling pathways (Table 1B). In Table 1, “15” represents baseline, “35” represents immediately after listening to the CD, and “50” represents 15 minutes after listening to the CD.

The analysis also identified several pathways that were significantly downregulated in M versus N1 and M versus N2 only (long term pattern). These pathways are significantly linked to immune response (TCR signaling, Antigen Processing and Presentation, IL10 signaling), cell cycle (apoptotic pathways) as well as stress-related pathways (stress pathway, P38 MAPK). In upregulated long-term patterns, we identified overrepresentation of pathways linked to telomere maintenance and cardiac muscle contraction.

Table 1A and 1B show a list of pathways with significant progressive relaxation response patterns:

TABLE 1A
Progressive Downregulated

| | N1 vs N2 | | | N1 vs M | | | N2 vs M | | |
|---|---|---|---|---|---|---|---|---|---|
| Name | 15 | 35 | 50 | 15 | 35 | 50 | 15 | 35 | 50 |
| ADHERENS JUNCTIONS INTERACTIONS | ◯ | ◯ | | | ◯ | | | | ◯ |
| 0 CL DEPENDENT NEUROTRANSMITTER TRANSPORTERS | ◯ | | ◯ | ◯ | | | ◯ | | |
| STEROID HORMONE BIOSYNTHESIS | ◯ | ◯ | | ◯ | | | ◯ | | |
| CYTOCHROME P450 ARRANGED BY SUBSTRATE TYPE | ◯ | ◯ | | ◯ | | | ◯ | | |
| PHASE 1 FUNCTIO0LIZATION OF COMPOUNDS | ◯ | ◯ | | ◯ | | | ◯ | | |
| BIOLOGICAL OXIDATIONS | ◯ | ◯ | | ◯ | | | ◯ | | |
| STEROID HORMONES | ◯ | ◯ | | ◯ | | | ◯ | | |
| HORMONE BIOSYNTHESIS | ◯ | ◯ | | ◯ | | | ◯ | | |
| XENOBIOTICS | ◯ | ◯ | | ◯ | | | ◯ | | |
| ELECTRON TRANSPORT CHAIN | | ◯ | ◯ | | | ◯ | | | ◯ |
| PARKINSONS DISEASE | | ◯ | ◯ | | | ◯ | | | ◯ |
| GLUCOSE REGULATION OF INSULIN SECRETION | | ◯ | ◯ | | | ◯ | | | ◯ |
| HUNTINGTONS DISEASE | | ◯ | ◯ | | | ◯ | | | ◯ |
| ALZHEIMERS DISEASE | | ◯ | ◯ | | | ◯ | | | ◯ |
| DIABETES PATHWAYS | | ◯ | ◯ | | | ◯ | | | ◯ |
| INTEGRATION OF ENERGY METABOLISM | | ◯ | ◯ | | | ◯ | | | ◯ |
| REGULATION OF INSULIN SECRETION | | ◯ | ◯ | | | ◯ | | | ◯ |
| SHCMEDIATED CASCADE | ◯ | ◯ | | | | | ◯ | | ◯ |
| FGFR LIGAND BINDING AND ACTIVATION | ◯ | ◯ | | | | | ◯ | | ◯ |
| PHOSPHOLIPASE CMEDIATED CASCADE | ◯ | ◯ | | | | | ◯ | | ◯ |
| FRS2MEDIATED CASCADE | ◯ | ◯ | | | | | ◯ | | ◯ |
| DOWNSTREAM SIG0LING OF ACTIVATED FGFR | ◯ | ◯ | | | | | ◯ | | ◯ |
| CELL CELL ADHESION SYSTEMS | ◯ | ◯ | | | ◯ | | ◯ | | ◯ |
| CELL JUNCTION ORGANIZATION | ◯ | ◯ | | | ◯ | | ◯ | | ◯ |
| RETINOL METABOLISM | ◯ | ◯ | | ◯ | | | ◯ | | ◯ |
| DRUG METABOLISM CYTOCHROME P450 | ◯ | ◯ | | ◯ | | | ◯ | ◯ | |
| SLC MEDIATED TRANSMEMBRANE TRANSPORT | ◯ | | ◯ | ◯ | | | ◯ | ◯ | |

TABLE 1B
Progressive Downregulated

| | N1 vs N2 | | | N1 vs M | | | N2 vs M | | |
|---|---|---|---|---|---|---|---|---|---|
| Name | 15 | 35 | 50 | 15 | 35 | 50 | 15 | 35 | 50 |
| VIP PATHWAY | ◯ | | ◯ | | | | ◯ | | |
| FURTHER PLATELET RELEASATE | ◯ | | ◯ | | | | ◯ | | |
| NEUROTROPHIN SIGNALING PATHWAY | ◯ | | ◯ | | | | ◯ | | |
| RELA PATHWAY | ◯ | ◯ | | | | | ◯ | | |
| TNFR2 PATHWAY | ◯ | ◯ | | | | | ◯ | | |
| NFKB PATHWAY | ◯ | ◯ | | | | | ◯ | | |
| GRAFT VERSUS HOST DISEASE | ◯ | | ◯ | ◯ | | | ◯ | | |
| GPCR PATHWAY | ◯ | | | | | | ◯ | | |
| CALCINEURIN PATHWAY | ◯ | | | | | | ◯ | | |
| TRANSPORT OF MATURE MR0 DERIVED FROM AN INTRON | ◯ | | | | | | ◯ | | |
| CONTAINING TRAN | | | | | | | | | |
| MRNA 3 END PROCESSING | ◯ | | | | | | ◯ | | |
| NUCLEAR IMPORT OF REV PROTEIN | ◯ | | | | | | ◯ | | |
| VPR MEDIATED NUCLEAR IMPORT OF PICS | ◯ | | | | | | ◯ | | |
| PROCESSING OF CAPPED INTRON CONTAINING PRE MRNA | ◯ | | | | | | ◯ | | |
| TRANSPORT OF THE SLBP INDEPENDENT MATURE MRNA | ◯ | | | | | | ◯ | | |
| TRANSPORT OF RIBONUCLEOPROTEINS INTO THE HOST NUCLEUS | ◯ | | | | | | ◯ | | |
| NEP NS2 INTERACTS WITH THE CELLULAR EXPORT MACHINERY | ◯ | | | | | | ◯ | | |
| GLUCOSE TRANSPORT | ◯ | | | | ◯ | | ◯ | | |
| GCR PATHWAY | ◯ | | ◯ | | | | ◯ | ◯ | |
| IL7 PATHWAY | ◯ | | ◯ | ◯ | | | ◯ | ◯ | |
| DOWNSTREAM TCR SIG0LING | ◯ | | ◯ | ◯ | | | ◯ | ◯ | |
| CSK PATHWAY | ◯ | | ◯ | ◯ | | | ◯ | ◯ | |
| MEMBRANE TRAFFICKING | ◯ | ◯ | | ◯ | | | ◯ | ◯ | |

Table 1A shows upregulated pathways. Table 1B shows downregulated pathways. The columns represent time points of comparison between two groups and rows represent the biological pathways. The solid dots denote significantly affected pathways (False Discovery Rate <25%) at a particular time point.

### Upregulated Progressive Changes Induced by RR are Linked to Energy Production in Mitochondria

To identify a set of focus gene hubs and RR signatures that may be playing a critical role in relaxation response, we applied systems biology strategies to generate interactive gene networks. For upregulated progressive patterns, focus gene hubs were identified from 27 pathways depicting changes from N1 versus N2 as well as N1 versus M, but with greater significant levels for the former comparison. These pathways are related to energy production (i.e. Electron Transport Chain, Integration of Energy Metabolism), metabolism (i.e. Retinol Metabolism, Hormone Biosynthesis), growth factors (i.e. FGFR ligand binding and activation, FRS2 mediated cascade) and glucose regulation (i.e. Regulation of Insulin Secretion). Interestingly, the genes with progressive induction patterns depicted significant enrichment in pathways linked to diabetes and neurological diseases (i.e. Parkinson and Alzheimer's Disease).

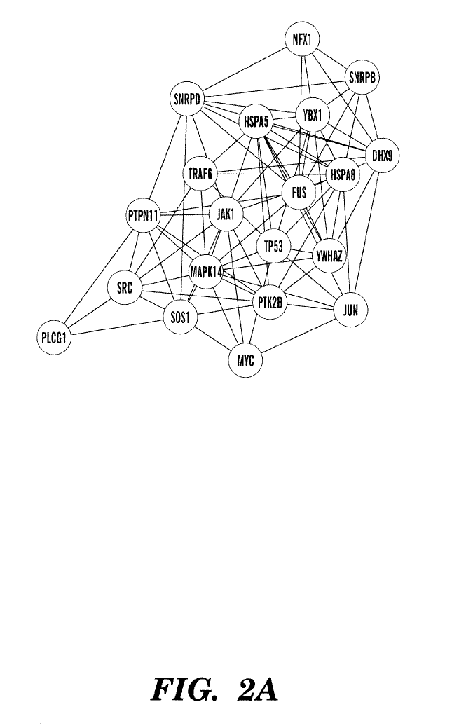

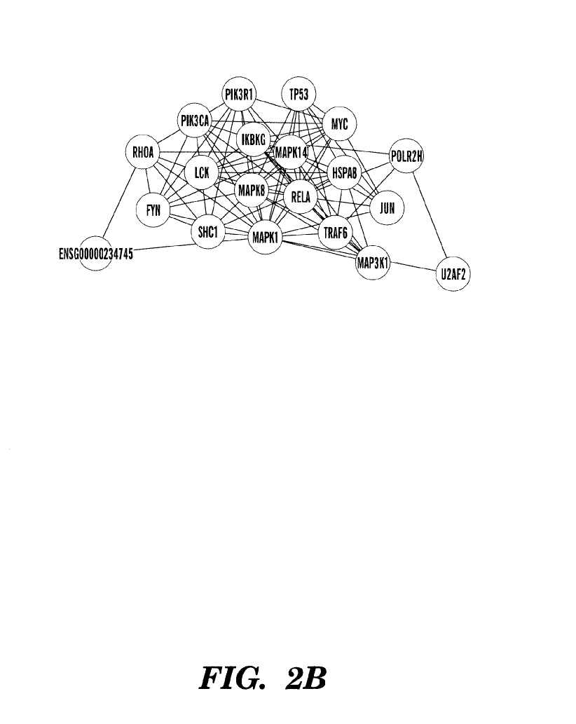

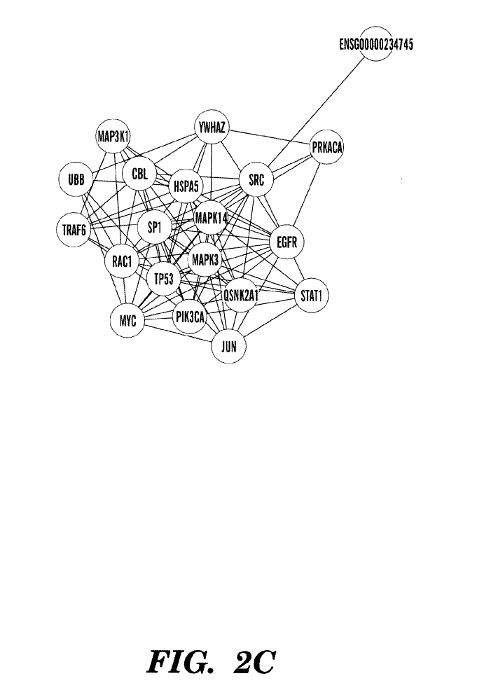

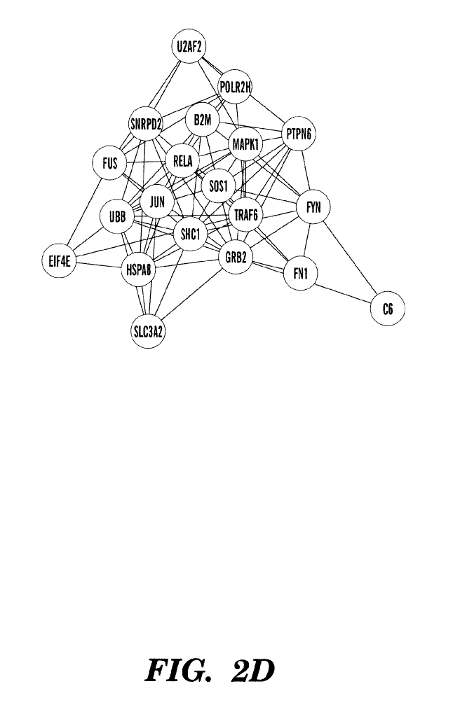

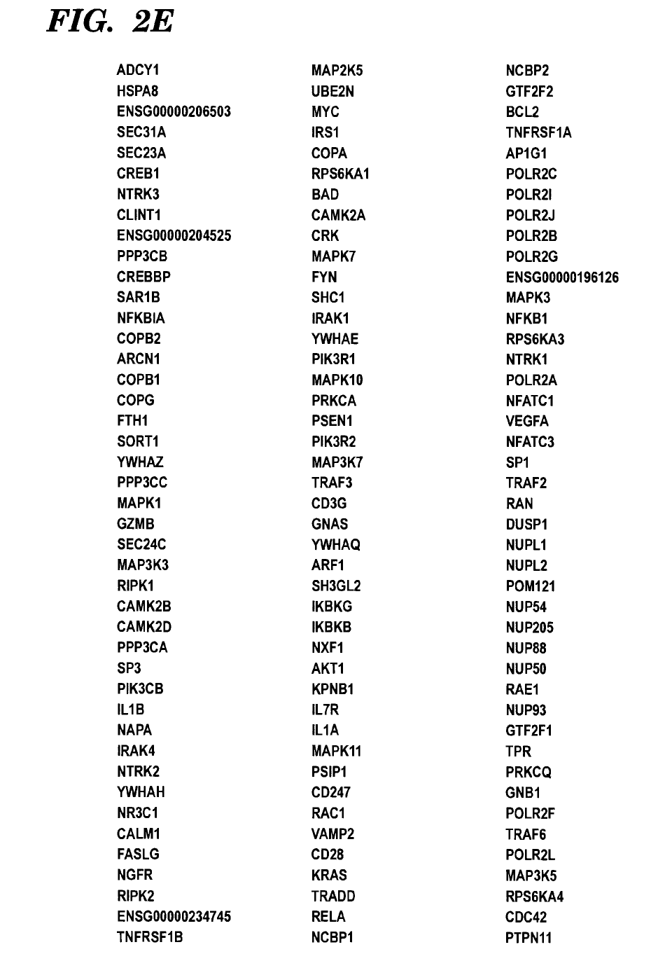

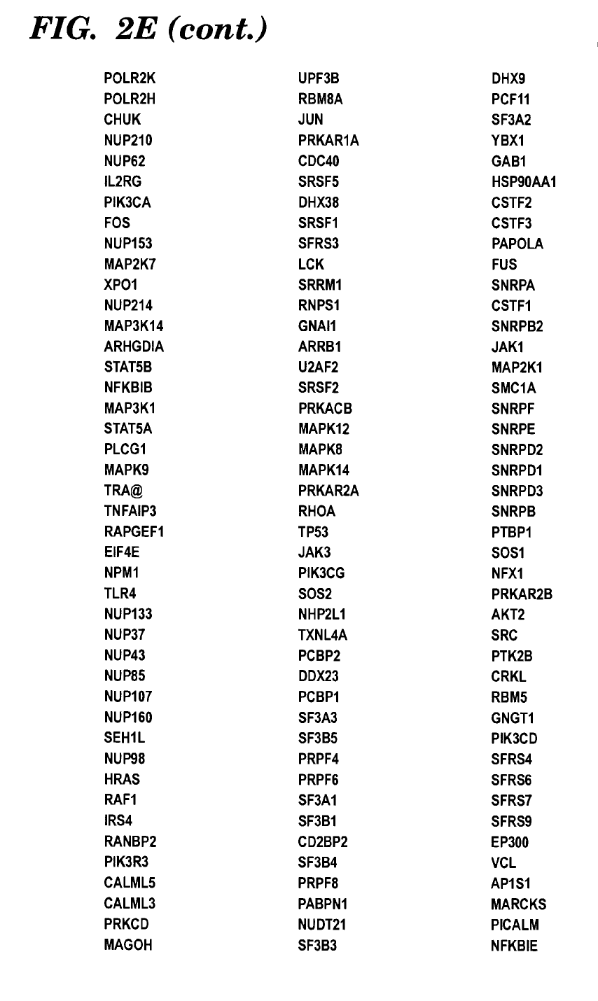

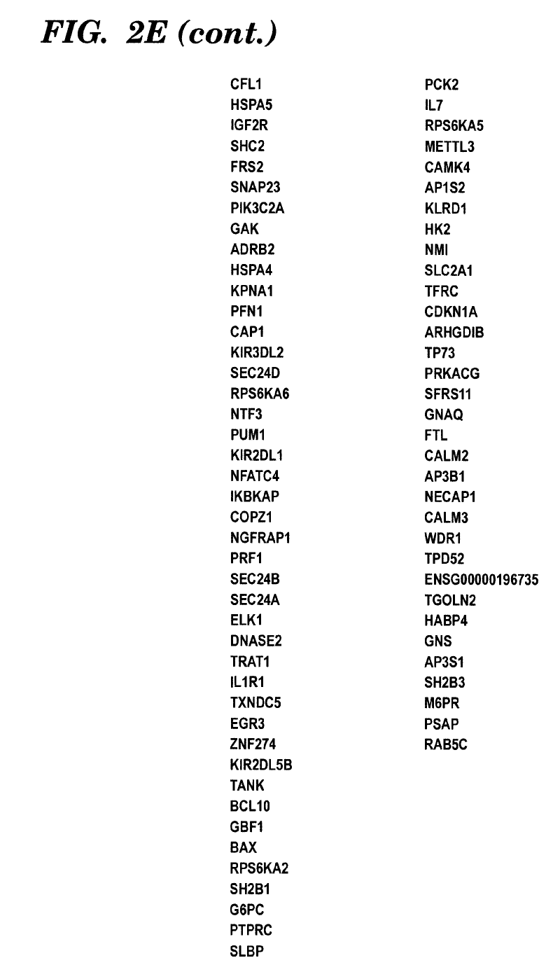

The Cytoscape program was used to generate interactive networks from enriched pathways depicting progressive, long term or short-term patterns. The networks were generated mainly on the basis of direct physical or biochemical protein-protein interactions, with a relatively small number of experimentally verified protein-DNA or protein-RNA interactions. These interactive networks were further analyzed to identify network hubs and bottlenecks, which may represent the key regulatory nodes in the network. The analysis identified a sub-network of the top 20 focus gene hubs (progressive RR signature). ATP synthase (ATP5C1), cAMP-dependent protein kinase (PRKACA) and Insulin Receptor (INSR) genes are the top focus genes hubs in pathways depicting progressive upregulated patterns (FIG. 2). All these focus gene hubs are linked to energy production and usage in mitochondria.

In a similar manner, systems biology analysis of pathways with short- and long-term RR patterns showed enrichment of G-Protein signaling molecules. The analysis of pathways depicting long-term RR effects also showed genes linked with DNA stability, recombination and repair (i.e. HIST1H2BC, PCNA) as focus genes. These genes play a critical role in telomere stability and maintenance.

### Downregulated Gene Expression Changes Induced by RR are Linked to Alteration of NF-κB Activity

Interactive network and focus hub investigation analysis was also performed on 23 pathways depicting the progressive downregulated pattern. These pathways are mainly related to inflammation (i.e. NF-κB, TNFR2, RELA), immune response (i.e. IL7, CCR5), T cell signaling (i.e. Downstream TCR signaling) and mRNA Processing. The progressively downregulated genes depict significant enrichment in pathways linked to Graft vs. Host Disease (GVHD). The focus hub investigation analysis on the progressive down-regulated network identified many molecules related to NF-κB activity including MAPK14, MYC, PTPKB2, TP53, and TRAF6 as focus gene hubs (FIG. 3A).

Additionally, systems biology analysis of short term pathways as well as long term RR effect pathways demonstrated enrichment of NF-κB activity related molecules (e.g. RELA, TRAF6, MAPK14, MAPK11, TP53, MYC). To better understand the molecular mechanism of RR and identify the most critical focus genes, we merged the long, short and progressive system biology networks and investigated the focus hubs in this integrated network. The network of the top 20 focus hubs (RR signature) clearly depicted enrichment for NF-κB upstream and downstream target molecules indicating its critical role in RR (FIG. 3B).

### Discussion

Mitochondria use a complex signaling cascade to sense internal and external pertubations and to finely tune and manipulate bioenergetic, immune, oxidative and apoptotic pathways in an effort to maintain stability of physiological processes in the face of change, i.e., allostasis. Psychosocial stress has been shown to cause mitochondrial oxidative stress with subsequent metabolic wear and tear in the above pathways leading to what is called allostatic loading, which has been associated with disease vulnerability and aging. On the basis of temporal analysis of GEP, GSEA, pathway analyses and identification of critical focus gene regulators, the data presented suggest that the practice of RR can progressively enhance energy metabolism and insulin secretion while diminishing T-cell signaling and long term as well as progressive effect of RR practice can modulate downward inflammatory and stress-related pathways. In addition, long term RR practice appears to be linked to telomere maintenance. This is of interest because telomere dysfunction can cause disruption of mitochondrial regulators and cause mitochondrial compromise that ends in apoptosis. Taken together this study suggests that RR practice can progressively enhance mitochondrial resiliency, which may reduce allostatic loading and become responsible at the cellular level for the downstream health benefits of reducing psychosocial stress and enhancing overall resiliency.

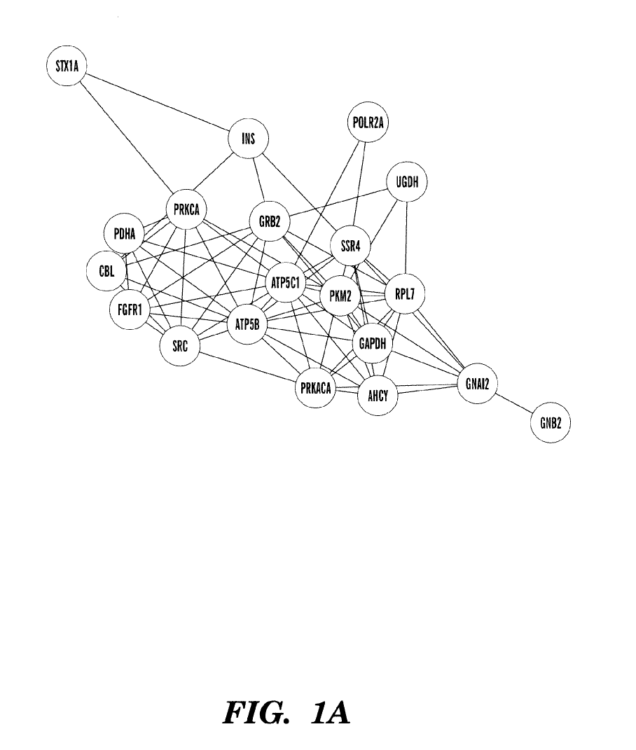

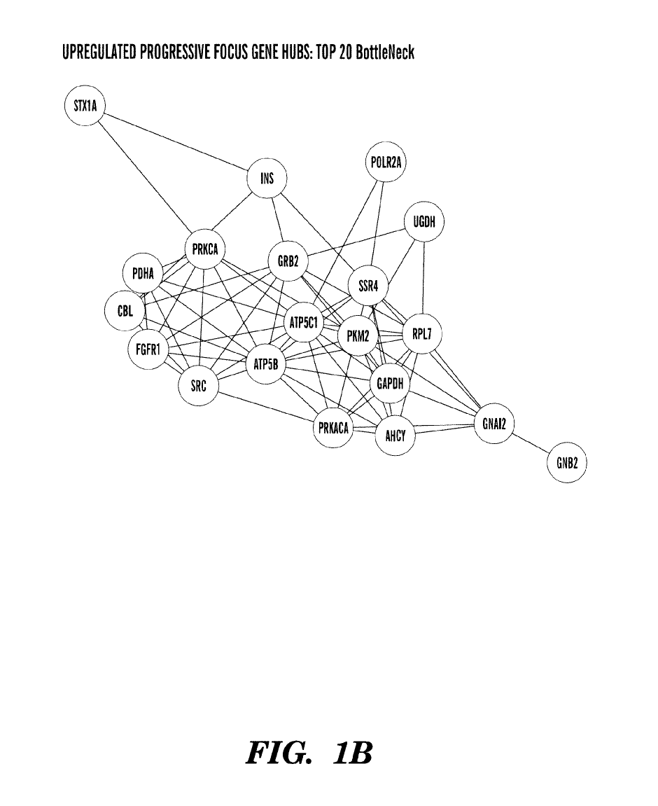

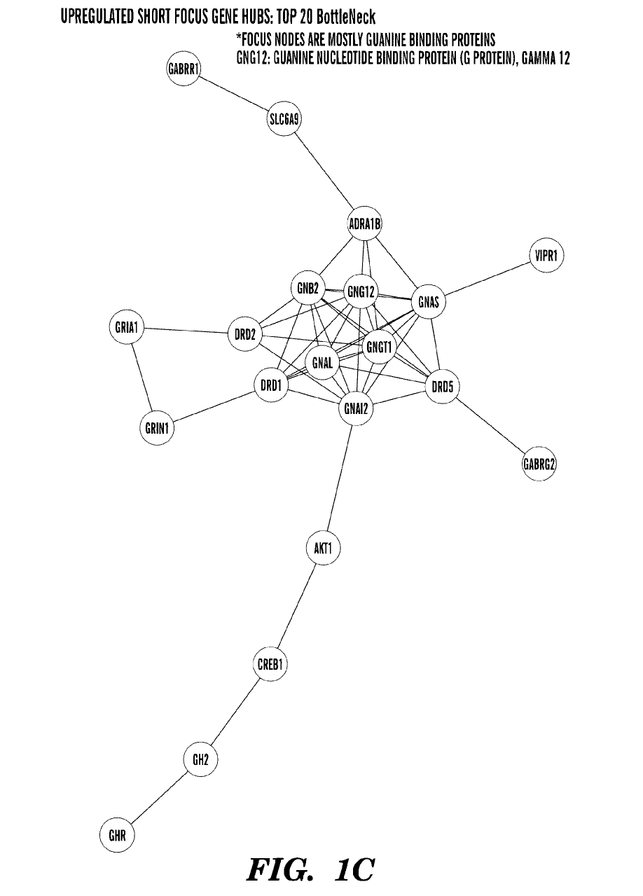

TABLE 2
Upregulated genes (progressive focus) in relaxation response
(shown in FIG. 1A)

| | |
|---|---|
| STX1A | POLR2A |
| INS | SSR4 |
| PRKCA | PKM2 |
| PDHA1 | PRKACA |
| CBL | UGDH |
| FGFR1 | RPL7 |
| SRC | GAPDH |
| ATP5B | AHCY |
| ATP5C1 | GNAI2 |
| GRB2 | GNB2 |

TABLE 3
Upregulated genes (long focus) in relaxation response
(shown in FIG. 1B)

| | |
|---|---|
| CYC2 | GNB2 |
| GNB1 | GNG12 |
| F2 | IL8 |
| CACNA1C | POU2F1 |
| PARD3 | POLR2K |
| RARD6A | BRF1 |
| INADL | RUVBL1 |
| HIST1H2BC | H2AFX |
| PLG | PCNA |
| CXCL2 | RPA1 |

TABLE 4
Upregulated genes (short focus) in relaxation response
(shown in FIG. 1C)

| | |
|---|---|
| GABRR1 | DRD1 |
| SLC6A9 | GNAL |
| ADRA1B | GNGT1 |
| GNB2 | GNAI2 |
| GNG12 | AKT1 |
| GNAS | CREB1 |
| VIPR1 | GH2 |
| GRIA1 | GHR |
| GRIN1 | DRD5 |
| DRD2 | GABRG2 |

TABLE 5
Downregulated genes (progressive) in relaxation response
(shown in FIG. 2A)

| | |
|---|---|
| NFX1 | PTPN11 |
| SNRPD2 | SRC |
| HSPA5 | PLCG1 |
| YBX1 | SOS1 |
| SNRPB | MAPK14 |
| DHX9 | TP53 |
| TRAF6 | YWHAZ |
| FUS | PTK2B |
| HSPA8 | JUN |
| JAK1 | MYC |

TABLE 6
Downregulated genes (merged top 20, short, long, progressive)
in relaxation response (in FIG. 2B)

| | |
|---|---|
| PIK3R1 | RHOA |
| TP53 | LCK |
| MYC | MAPK8 |
| PIK3CA | MAPK1 |
| IKBKG | TRAF6 |
| MAPK14 | FYN |
| HSPA8 | SHC1 |
| POLR2H | MAPK31 |
| JUN | U2AF2 |
| RELA | ENSGOOOOO234745 |

TABLE 7
Downregulated genes (long) in relaxation response
(shown in FIG. 2C)

| | |
|---|---|
| ENSGOOOOO234745 | EGFR |
| MAP3K1 | TRAF6 |
| YWHAZ | RAC1 |
| PRKACA | TP53 |
| SRC | MYC |
| CBL | JUN |
| UBB | STAT1 |
| HSPA5 | PIK3CA |
| MAPK14 | CSNK2A1 |
| SP1 | MAPK3 |

TABLE 8
Downregulated genes (short) in relaxation response
(shown in FIG. 2D)

| | |
|---|---|
| U2AF2 | FN1 |
| POLR2H | FUS |
| B2M | JUN |
| SNRPD2 | SHC1 |
| RELA | GBR2 |
| MAPK1 | C6 |
| PTPN6 | UBB |
| SOS1 | EIF4E |
| TRAF6 | HSPA8 |
| FYN | SLC3A2 |

### Example 2

### Relaxation Response Induces Temporal Transcriptome Changes in Energy Metabolism, Insulin Secretion and Inflammatory Pathways

The relaxation response (RR) is a physiological and psychological state opposite to the stress or flight-or-flight response (1-3). Results from rigorous research studies indicate the ability of various mind-body interventions to reduce chronic stress and enhance wellness through induction of the RR (1, 4). Several studies also reported that elicitation of the RR is an effective therapeutic intervention to counteract the adverse clinical effects of stress in disorders that include: hypertension (5); anxiety (6, 7); insomnia (8, 9); diabetes (10); rheumatoid arthritis (11); and aging (12, 13).

The RR is elicited when an individual focuses on a word, sound, phrase, repetitive prayer, or movement, and disregards everyday thoughts (2). These two steps break the train of everyday thinking. Millennia-old mind-body approaches that elicit the RR include: various forms of meditation (e.g., mindfulness meditation and transcendental meditation); different practices of yoga (e.g., Vipassana and Kundalini); Tai Chi; Qi Gong; progressive muscle relaxation; biofeedback; and breathing exercises (14). Elicitation of the RR is associated with coordinated biochemical changes, characterized by decreased oxygen consumption (15), carbon dioxide elimination, blood pressure, heart and respiratory rate (16, 17), and norepinephrine responsivity (18), as well as increased heart rate variability (18, 19), as well as increased heart rate variability (19) and alterations in cortical and subcortical brain regions (20, 21).

A previous study provided the first evidence that RR practice in healthy subjects at rest results in genomic expression alterations when comparing long-term RR practitioners to novices before and after their short-term RR training (22). Specifically, sustained expression changes in genes significantly linked to oxidative phosphorylation, antigen processing and presentation, and apoptosis were identified in both short-term (N2) and long-term RR (M) practitioners at rest when compared to novices (N1) (22). It is noteworthy that these changes were particularly evident among long-term practitioners suggesting that RR induces lasting changes of the base level of expression for a selected set of genes that persist over prolonged periods of time (22).

Regular daily practice of techniques that can be used to elicit the RR are often recommended for sustaining its beneficial effects. The immediate psychological and physiological effects from one session of RR-eliciting practice have been reported (1, 23). No study so far, however, has examined the acute changes in gene expression within one session of RR-eliciting practice and the impact of the length of previous practices on these immediate effects. In this current study, the rapid temporal gene expression changes were determined among these same study subjects using blood samples collected at 3 successive time points during a single RR practice session, which included listening to a 20-minute RR-eliciting CD. It was recently reported that these healthy subjects evoked psychobiological changes during one session of RR practice and that the psychological reactions correlate with biological changes only among long-term practitioners (23). It was hypothesized that in both long-term and short-term practitioners, one session of RR practice would evoke changes in gene expression that would be linked to a select set of biological pathways. It was further hypothesized that the changes would be more profound among long-term practitioners than those with short-term practice. In this study, systems biology and interactive network analyses were employed to identify focus gene hubs of RR. Identification of these gene hubs, which are focal points or critical molecules in broad networks of interacting genes, could provide an empiric foundation for future investigations of genomic mechanisms of RR practices in specific clinical conditions. In addition, the investigation of the genomic expression changes that might occur during one session of RR practice will likely provide the scientific rationale for daily practice of RR elicitation, which is the common practice method recommended and followed by practitioners.

### Methods

Samples.

Twenty-six healthy practitioners of various RR-eliciting techniques (including several types of meditation, Yoga, and repetitive prayer) participated in the time series study. Twenty-seven individuals without any prior RR-eliciting experience served as controls (N group; N1=27). Novice subjects had 8-weeks of RR training, listened to a 20-minute RR-eliciting CD daily and are classified as N2 after 8 week of training (N2=26). Whole blood (5 ml) was collected at 0, 20 and 35 minutes each of the study participants (FIG. 5). Total RNA extraction and purification was performed using previously described protocols (22).

Transcriptional Profiling.

For transcriptional profiling, the Affymetrix human genome high throughput arrays plates with 96 arrays (HT U133A), containing more than 22,000 transcripts, was used. Microarray analysis was conducted by the BIDMC Genomics and Proteomics Center at the Beth Israel Deaconess Medical Center according to previously described protocols for total RNA extraction and purification, complementary DNA (cDNA) synthesis, in vitro transcription for production of biotin-labeled cRNA, hybridization of cRNA with human genome HT U133A Affymetrix plates, and scanning of image output file. The quality of scanned arrays images were determined on the basis of background values, percent present calls, scaling factors, and 3′-5′ ratio of actin and GAPDH using the SimpleAffy package for R (27).

Scanned array images were analyzed by dChip (54). The raw probe level data was normalized using smoothing-spline invariant set method, and the signal value for each transcript was summarized using the PM-only based signal modeling algorithm in which the signal value corresponds to the absolute level of expression of a transcript (54). To calculate model based expression signal values, array and probe outliers were interrogated and image spikes were treated as signal outliers. The outlier detection was carried out using the dChip outlier detection algorithm. A chip was considered to be an outlier if the probe, signal or array outlier percentage exceeded a threshold of 10%. No chips were found to be outliers.

Data Analysis.

The differentially expressed genes among the two classes or two time points were defined using a random-variance t-test. The random-variance t-test is an improvement over the standard separate t-test as it permits sharing information among genes about within-class variation without assuming that all genes have the same variance. For the comparison of N1 vs. M or N2 vs. M at any time point unpaired univariate t-test was used. The paired t-test was used for the time dependence within each group or comparison of N1 group with N2 group. Genes were considered statistically significant if their p value was less than 0.01. P-values for significance were computed based on 1,000 random permutations, at a nominal significance level of each univariate test of 0.01.

To identify time and group dependent patterns from differentially expressed genes, the Self Organizing Map (SOM) clustering technique was adopted (26). SOM allows the grouping of gene expression patterns into an imposed structure in which adjacent clusters are related, thereby identifying sets of genes that follow certain expression patterns across different conditions. SOM clustering was performed on transcript expression values using Pearson correlation coefficient based distance metrics and a target of 18 groups.

Gene Ontology (GO) Enrichment Analysis.

To identify the over-represented GO categories in the different gene expression patterns obtained from SOM clustering, the Biological Processes and Molecular Functions Enrichment Analysis available from the Database for Annotation, Visualization and Integrated Discovery (DAVID) was used (28). DAVID is an online implementation of EASE software that produces a list of over-represented categories using jackknife iterative resampling of the Fisher exact probabilities. A p-value gets assigned to each category on the basis of enrichments. Smaller p-value reflects increasing confidence in over-representation. The GO categories with p-value <0.05 were considered significant.

Pathways and Interactive Network Analysis.

Interactive networks and pathways were analyzed for different patterns identified using SOM analysis of differentially expressed genes using the commercial system biology oriented package Ingenuity Pathways Analysis (IPA 4.0) (http://www.ingenuity.com/). The knowledge base of this software consists of ontology and network models derived by systematically exploring the peer reviewed scientific literature. It calculates the P value using Fisher Exact test for each network and pathway according to the fit of user's data to the IPA database (55).(55). It displays the results as a score (−log P value) indicating the likelihood of a gene to be found in a network or pathways by random chance. For example, a network achieving a score of 2 has at least 99% confidence of not being generated by chance alone.

Gene Set Enrichment Analysis.

Gene Set Enrichment Analysis (GSEA) was used to determine whether an a priori defined sets of genes showed statistically significant, concordant differences between 2 groups (N2 vs. N1, and M vs. N1) or two time points (15 minutes vs. 35 minutes, 15 minutes vs. 50 minutes) in the context of known biological sets. GSEA is more powerful than conventional single-gene methods for studying the effects of interventions such as RR in which many genes each make subtle contributions. GSEA calculates an enrichment score using the Kolmogorov-Smirnov test (KS-test) for determining whether a rank-ordered list of genes for a particular comparison of interest is enriched in a biologically related geneset. The enrichment analysis was performed using the 880 canonical pathways derived from MSigDB2.0 (24, 25). The enriched gene sets had nominal p-value (NPV) less than 5% and a False Discovery Rate (FDR) less than 25% after 500 random permutations. These criteria ensured that there was minimal chance of identifying false positives.

The genes from enriched pathways were merged into functional modules on the basis of overlap of significantly enriched genes using enrichment map plugin (56) in cytoscape: An Open Source Platform for Complex Network Analysis and Visualization (57). Genes with significant overlap (70% common genes) were considered neighbor and substitutable with each other. The patterns in significantly enrichment genesets from different comparisons (e.g. N vs. M, N1 vs. N2, 15 min vs. 35 min, 15 min vs. 50 min) were identified by developing a dotplots in lattice package. The interesting selected patterns were divided into the following major groups: i) long-term effects (M vs. N₁ or M vs. N₂), ii) progressive effects (changes which occur in both M and N₂ compared to N₁, and are of greater significance in M). Furthermore, by virtue of having three time points, it was possible to describe constitutive changes (present at all three time points), acute changes (only present immediately after RR-elicitation) and delayed changes (only present 15 minutes after RR completion). Pattern classification details are discussed below.

Systems Biology Analysis.

Genes of pathways from various patterns were selected and an integrated network was generated using known Protein-Protein, Protein-DNA and Protein-RNA interactions. The interaction information was obtained using literature search, information from knowledge base databases such as MIPS, DIPS, HPRD and ingenuity systems (58, 59). Networks were analyzed using the cyto-Hubba plug-in for Cytoscape 2.8 platform to identify network hubs and bottlenecks, which may represent the key regulatory nodes in the network (60). The network consisting of top 20 focus gene hubs was considered as the RR core signature network.

Classification of GSEA Enrichment Patterns.

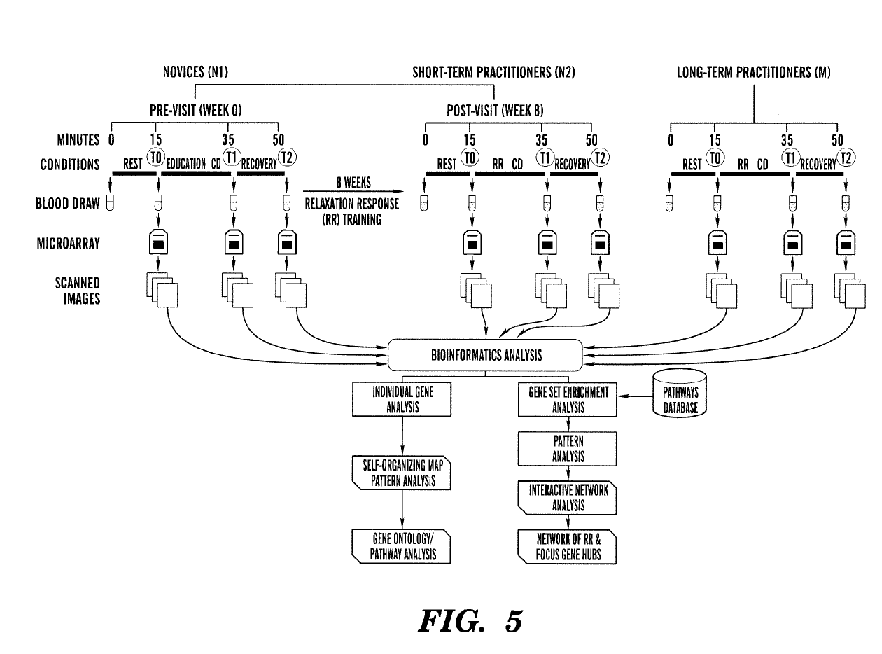

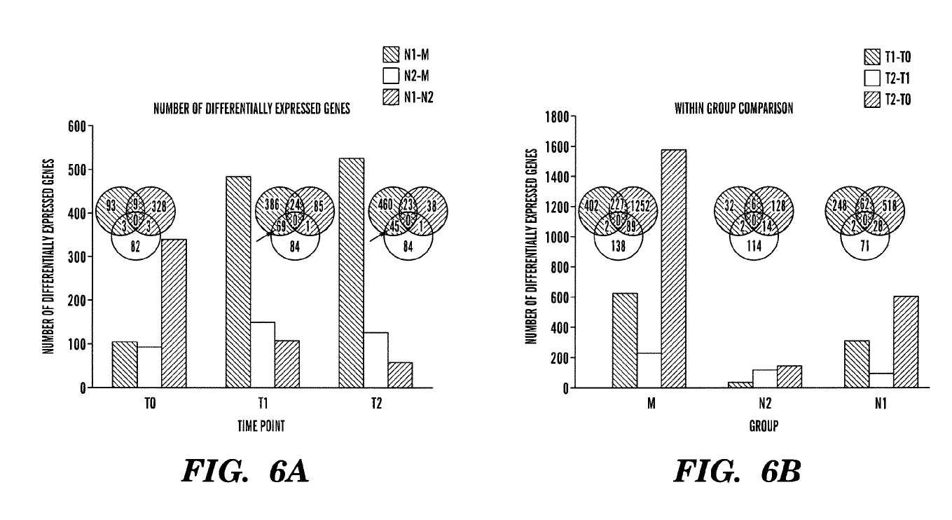

The patterns are explained in detail below:
i) Progressive I patterns: The Progressive-I Upregulated pattern consisted of gene sets that were significantly enriched in both N2 and M as compared to N1 and with greater enrichments in M (i.e., more time points with significant enrichments in M compared to N1 and N2) (FIG. 6A, solid dots indicating significant group differences). In addition to these across group differences at each time point, most gene sets also showed significant changes across time points within each group (FIG. 6A, asterisks indicating significant time difference). Similarly, GSEA analysis identified Progressive-I Downregulated gene sets based on both across and within group comparisons.
ii) Progressive II patterns: GSEA identified pathways that depicted similar enrichments for M and N2 as compared to N1 at T1 and T2 in across group comparison. In addition to these across group differences at each time point, most gene sets also showed significant changes across time points within M group only. These pathways were classified as ‘Progressive II’ gene sets since the rapid enrichment within one session of RR practice can be evoked by short-term as well as long-term practitioners. In addition to across group changes, the M group was also able to depict the temporal changes in gene expression.
iii) Long-term patterns: GSEA identified pathways that were significantly upregulated in M at 2 or 3 time points compared to both N1 and N2, of which there were no significant group differences. In addition to these across group differences at each time point, most gene sets also showed significant changes across time points within M group only.

### Results

### Study Design

The study design was composed of both prospective and cross-sectional features (FIG. 5). The prospective aspect of the study involved enrolling 26 healthy subjects who had no prior RR-eliciting experience (Novices, N1). They then underwent 8 weeks of RR-eliciting training (Short-term Practitioners, N2). The cross-sectional aspect of the study involved enrolling another 26 healthy subjects who had significant prior experience of regular RR-eliciting practice for 4-20 years (Long-Term Practitioners, M) to be compared with novices either before or after their 8-week RR training. The details of the study recruitment and intervention have been described elsewhere (22).

All study subjects attended morning laboratory sessions, during which M and N2 listened to a 20-minute RR-eliciting CD and N1 listened to a 20-minute health education CD (control). Blood samples for gene expression profiling were collected immediately prior to (T0), immediately after (T1) and 15 minutes after (T2) listening to the respective 20-minute CD (FIG. 5). The gene expression changes were examined in peripheral blood mononuclear cells (PBMC) across the three time points (i.e., rapid or “temporal” changes) in the laboratory session. The study design allowed investigation of immediate transcriptome changes that were induced by one session of 20-minute RR practice and also examination of whether the changes differed by the length (long-term/years vs. short-term/weeks) of prior RR practice. Individual gene-based analysis and Gene Set Enrichment Analysis (GSEA) (24, 25) were performed to identify temporal gene expression changes that differentiate the three study groups (N1, N2, M) and the biological pathways associated with these gene expression changes (FIG. 5). To partition the differentially expressed genes into clusters with similar temporal expression patterns Self Organizing Map (SOM) (26) analysis was carried out. Rigorous bioinformatics and systems biology analysis was applied, including Gene Ontology (GO), pathway enrichment analysis and Interactive Network analysis to identify biological pathways and focus gene hubs that were affected most significantly during one session of RR practice (FIG. 5).

### RR Leads to Qualitative and Quantitative Temporal Transcriptome Changes: Individual Gene Analysis

Transcriptional profiling analysis was performed on all samples using the Affymetrix HT Human Genome U133A Array Plate. After stringent quality control analysis (27), group comparisons were conducted on the normalized gene expression data using permutated univariate t-tests to identify differentially expressed genes. The across-group comparison identified the sets of genes that were significantly differentially expressed between groups at each time point (FIG. 6A). Both Short-term (N2) and Long-Term (M) practitioners evoked significant temporal gene expression changes as compared to novices (N1) during one session of RR elicitation. A larger number of genes were differentially expressed between M and N1 groups than between M and N2 or N2 and N1 at T1, right after listening to the CD, and at T2, 15 minutes after listening to the CD (FIG. 6A), indicating that long-term practitioners exhibit more pronounced transcriptional changes in response to RR elicitation. These results corroborate with the previous observations that long term RR practitioners have more transcriptional changes as compared to short-term practitioners at rest (22).

It was next determined which differentially expressed genes were common to individual pairwise comparisons between groups (e.g., M vs. N1). These common genes are shown as the intersecting areas of the Venn diagrams in FIG. 6A. There was a significant overlap of differentially expressed genes between M vs. N1 and M vs. N2 at both T1 and T2 (69 and 45 transcripts, respectively, marked by arrows in FIG. 6A). These overlapping transcripts, corresponding to 39 well-annotated unique genes (when duplicates and multiple transcripts from the same gene were removed), represent temporal expression changes across the different groups (N1, N2, M) and across the different time points (T0, T1, T2). The expression of these genes showed a gradually decreasing or increasing trend from N1 to N2 to M (FIG. 6B). Most of these genes are significantly linked to immune response, apoptosis and cell death based on Gene Ontology (GO) Enrichment analysis (P value <0.05) (28). Similarly, the within-group comparison identified a number of genes that were differentially expressed across the three time points within each group. The long-term practitioners (M) demonstrated a rapid and more consistent response to RR elicitation in gene expression changes as indicated by a larger number of differentially expressed genes across the 3 time points (T1 vs. T0, T2 vs. T0, and T2 vs. T1;) than both the short-term practitioners (N2) and novices (N1). In addition, a larger number of genes showed significant expression changes from T2 to T0 than from T1 to T0, indicating possible lag effects of RR elicitation in the M group. In comparison to the M group, the N2 group had lower numbers of consistently differentially expressed genes, which may be a reflection of a greater heterogeneity among short-term practitioners with regard to proficiency in RR elicitation.

### RR Elicits Distinct Temporal Patterns of Differential Gene Expression: Self-Organizing Map (SOM) Analysis

To identify temporal gene expression patterns, SOM analysis was performed on all of the differentially expressed genes identified by the individual gene analysis above (26). Initially, differentially expressed genes were partitioned to 18 SOM patterns with different expression structures.

By way of example, temporal genomic expression patterns identified during one session of RR elicitation were analyzed. Genes that were differentially expressed either across or within groups comparisons at different time point were used as seed sets of genes for Self-Organizing Map (SOM) analysis. These differentially expressed genes were partitioned to 18 separate maps according to Pearson correlation coefficient based distance metrics. Each pattern represented a set of genes that depicted a similar expression pattern suggesting that they are biologically linked to a specific function. A box plot of the gene expression with X-axis representing time points and groups, and Y-axis representing scaled gene expression data from −1 to +1 was generated (not shown). The patterns were merged into 10 expression categories on the basis of similarities in expression patterns.

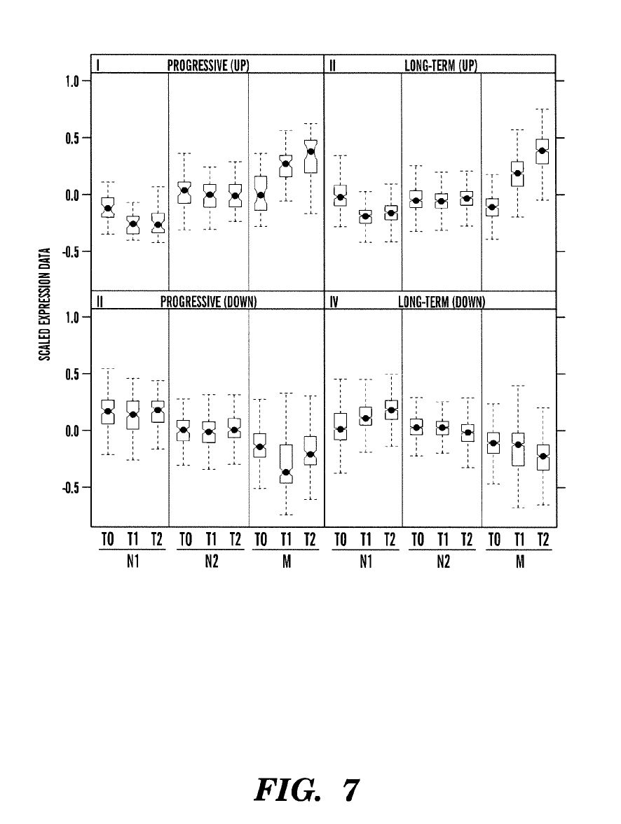

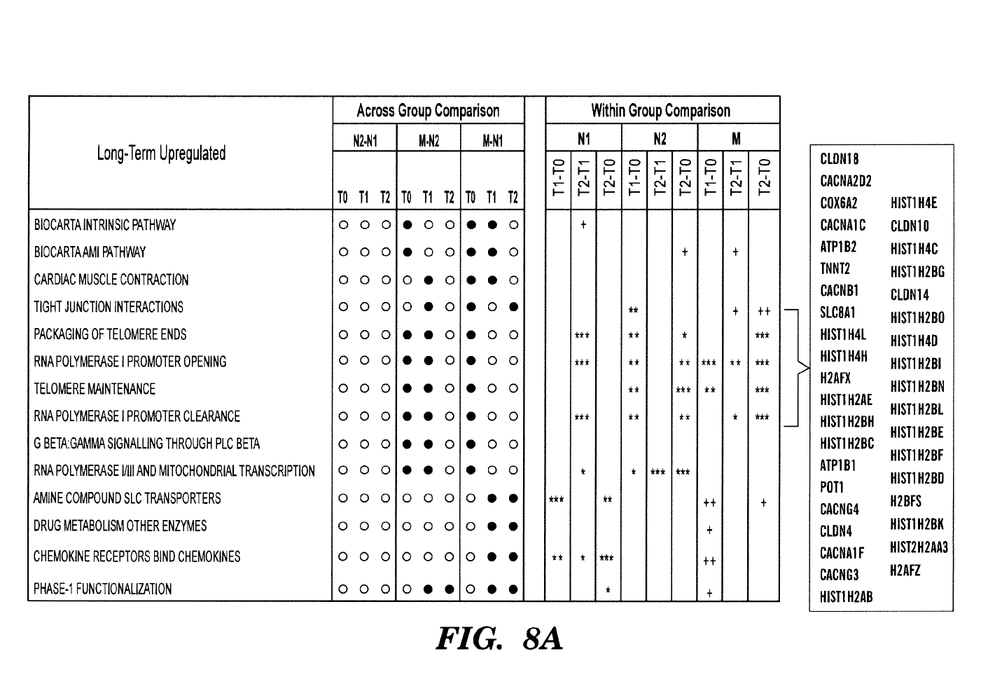

Based on their similarity in gene expression patterns, these 18 patterns were further merged into four distinct categories of related patterns. These 4 categories reflected temporal gene expression changes (i.e., over minutes, from T0-T2) associated with RR elicitation in relation to length of previous practice (i.e., weeks to years): 1. “Progressive” Upregulation; 2. “Progressive” Downregulation; 3. “Long-term” Upregulation; and 4. “Long-term” Downregulation. Representative sets of patterns from Long-term and Progressive categories are shown in FIG. 7, which displays the box plot of the standardized gene expression values at each time point for the three groups. A Progressive Upregulation pattern was defined as genes with gradual increases in expression according to the length of prior RR practices—none (group N1), weeks-long (group N2), vs. years-long (group M)—and time trend within one RR session. In other words, the gene expression values were the lowest in N1, higher in N2, and the highest in M, especially at T1 and T2. In addition, the gene expression increased sequentially from T0 to T1 to T2 in M, but little change was observed in N1 or N2 across the three time points (FIG. 7, Panel I). GO and Pathway Enrichment analyses using DAVID (28) identified genes with Progressive Upregulation patterns to be significantly linked to regulation of cell differentiation, cell adhesion, cell communication and transport, hormone stimulus, blood pressure, cAMP, metabolic processes, biological oxidation, GnRH signaling, neuroactive ligand-receptor interaction, type II diabetes mellitus, and oxidoreductase activity (shown below in Table 9).

TABLE 9
Gene-ontology enrichment analysis of progressive and long-term expression
patterns.

| | Enrichment | Related Processes |
|---|---|---|
| Progressive Upregulated | | |
| System Development | 10.6 | Nervous System Development |
| Cell Differentiation | 5.9 | Regulation Of Cell Proliferation |
| Protein Binding | 5.7 | |
| Transmission Of Nerve Impulse | 5.2 | |
| Cell Adhesion And Communication | 4.46 | |
| Regulation Of Transcription From | 4.2 | |
| Muscle Contraction | 4.05 | |
| Blood Vessel Morphogenesis | 3.8 | Vasculature Development |
| Steroid Hormone Receptor Activity | 3.5 | |
| Gland Development | 3.72 | |
| Regulation Of Transport | 3.36 | |
| Positive Regulation Of Transcription, | 3.25 | Regulation Of Transcription From |
| Glial Cell Differentiation | 3.06 | |
| Regulation Of Growth | 3 | |
| Response To Steroid Hormone | 2.8 | |
| Positive Regulation Of Phospholipase | 2.8 | |
| Response To Vitamin | 2.77 | |
| Second-Messenger-Mediated Signaling | 2.7 | Regulation Of Camp Metabolic |
| Regulation Of Systemic Arterial Blood | 2.4 | |
| Oxidoreductase Activity | 2.23 | |
| Response To Oxygen Levels | 2.23 | |
| Progressive Downregulated | | |
| Mrna Processing | 2.14 | |
| Intracellular Protein Transport | 2 | |
| Antigen Processing And Presentation | 1.6 | |
| Immune System Development | 1.5 | |
| Primary Metabolic Process | 1.5 | |
| Regulation Of DNA Binding | 1.5 | |
| Apoptosis | 1.36 | |
| Long-Term Upregulated | | |
| Protein Binding | 5.7 | |
| Glutamine Family Amino Acid | 2.6 | |
| Cellular Component Organization | 2.6 | |
| Cell-Matrix Adhesion | 2.26 | |
| Transcription Regulator Activity | 2.15 | |
| Regulation Of Neuronal Synaptic | 2.15 | |
| Multicellular Organismal Response To | 2 | |
| Cell Communication | 2 | |
| Cell Death | 1.96 | |
| Muscle Cell Differentiation | 1.94 | |
| Regulation Of Cytoskeleton | 1.79 | |
| Nervous System Development | 1.77 | |
| Membrane Organization | 1.67 | |
| Transcription Repressor Activity | 1.6 | |
| Amine Transport | 1.54 | |
| ATPase Activity | 1.53 | |
| Ubiquitin Cycle | 1.51 | |
| Long-Term Downregulated | | |
| Protein Binding | 11.3 | |
| Regulation Of Apoptosis | 3.8 | Cell Death, Apoptosis |
| Nuclear Transport | 3.6 | Protein, Macromolecule Transport |
| Mrna Processing | 2.8 | |
| Cellular Metabolic Process | 2.7 | |
| Positive Regulation Of Cellular Process | 2.4 | |
| JAK-STAT Cascade | 2.3 | |
| T Cell Activation | 2.1 | Lymphocyte Activation, Leukocyte |
| Response To Insulin Stimulus | 2.1 | Regulation Of Glucose Transport |
| Cell Cycle | 2 | Cell Division, Cell Cycle Process |
| Regulation Of DNA Binding | 2 | Negative Regulation Of NF-Kappa β |
| Response To Stress | 2 | |

In contrast, a distinct set of genes exhibited Progressive Downregulation patterns that had highest gene expression values in N1, lower in N2 and the lowest in M at all three time points. Furthermore, only the M group showed a decreasing time trend in gene expression from T0 to T1 (FIG. 7, Panel II). These genes are significantly linked to mRNA processing, intracellular protein transport, antigen processing and presentation, immune system, graft-versus-host disease, intestinal immune network for IgA production, and primary metabolism (Table 9).

Long-term Upregulation patterns were defined as those for which gene expression levels were elevated in M compared to both N1 and N2, for which there were few gene expression differences between the two at all three time points. Only the M group showed higher expression across the three time points as compared to N1 and N2 (FIG. 7, Panel III). Long-term upregulated genes are involved in adenosine triphosphate (ATP) activity, protein binding, cell matrix adhesion, defense response, amine transport, response to stress, gap junction, and muscle cell differentiation (Table 9).

Similarly, Long-term Downregulation patterns that contain genes with expression lower in M than both N1 and N2 were identified. In addition, only the M group exhibited a decreasing time trend across the 3 time points in gene expression. These genes are significantly associated with regulation of apoptosis, nuclear transport, metabolic processes, JAK-STAT cascade, T- and B-cell activation, regulation of cell cycle, insulin sensitivity, glucose transport, DNA replication, chemokine signaling, EGF signaling, and stress response (Table 9).

### RR Progressively Affected Energy Metabolism and Inflammation Pathways: Canonical Pathways Based on Gene Set Enrichment Analysis (GSEA)

While identification of gene expression differences and gene expression patterns based on individual-gene analysis described above is able to reveal a subset of statistically significant changes in gene expression, subtle (but statistically significant) gene expression differences in biologically- and functionally-linked genes in response to RR might be missed in this analysis. Gene Set Enrichment Analysis (GSEA) is a statistical approach that identifies enrichment of sets of differentially expressed genes that share a common biological function or regulation (24, 25).

GSEA was performed to identify enrichment of the statistically significantly affected gene sets that are associated with various pathways by comparing two groups at each time point as well as comparing changes across time points within each group. A False Discovery Rate (FDR) <25% was used to indicate significant group difference and a p value of <0.05 was used to indicate significant time difference. As previously, enriched pathways were categorized into 4 patterns based on the number of time points with group differences in gene set expressions: Progressive Upregulation, Progressive Downregulation, Long-term Upregulation and Long-term Downregulation. The progressive patterns were further categorized into Progressive I and Progressive II patterns on the basis of the similarity between M and N2 groups on genomic expression changes in comparison to N1.

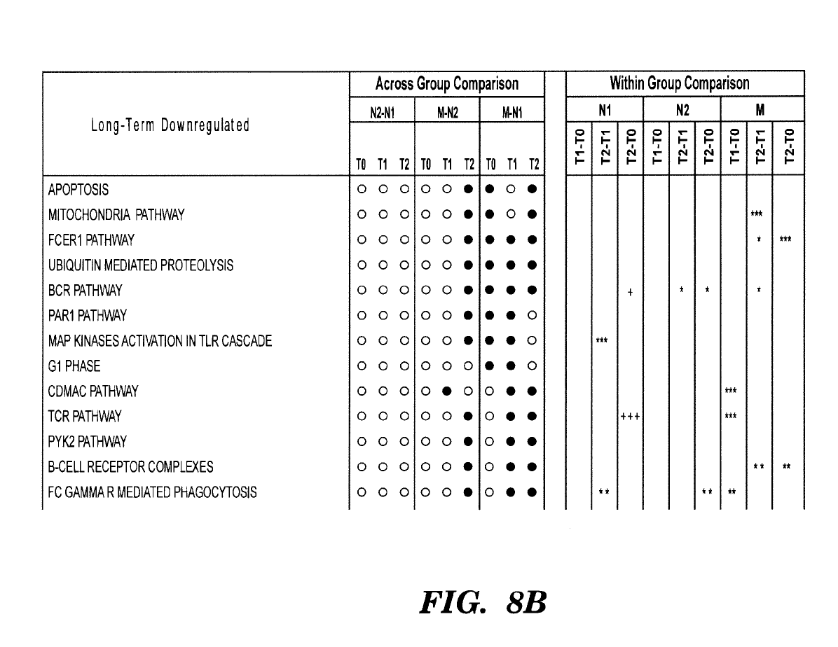

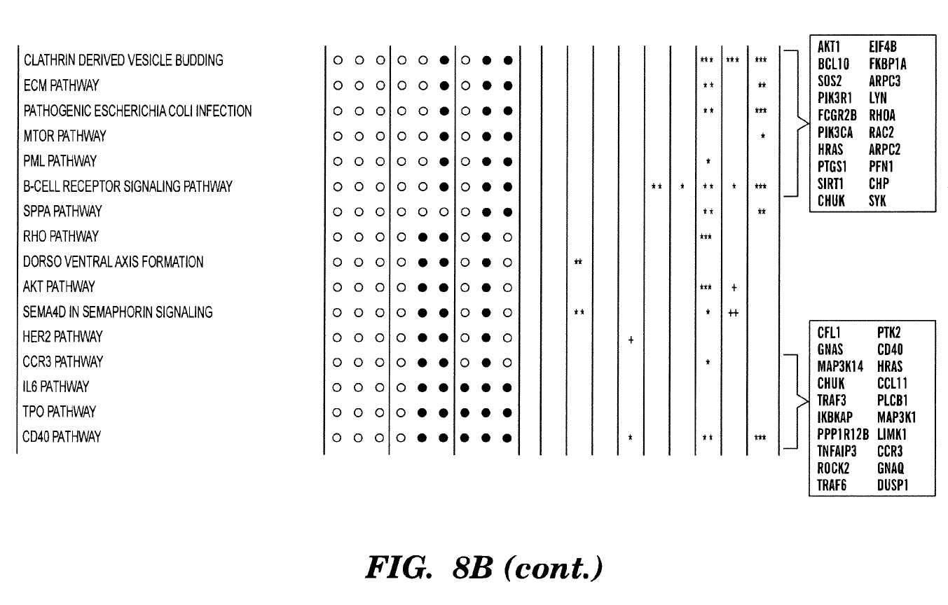

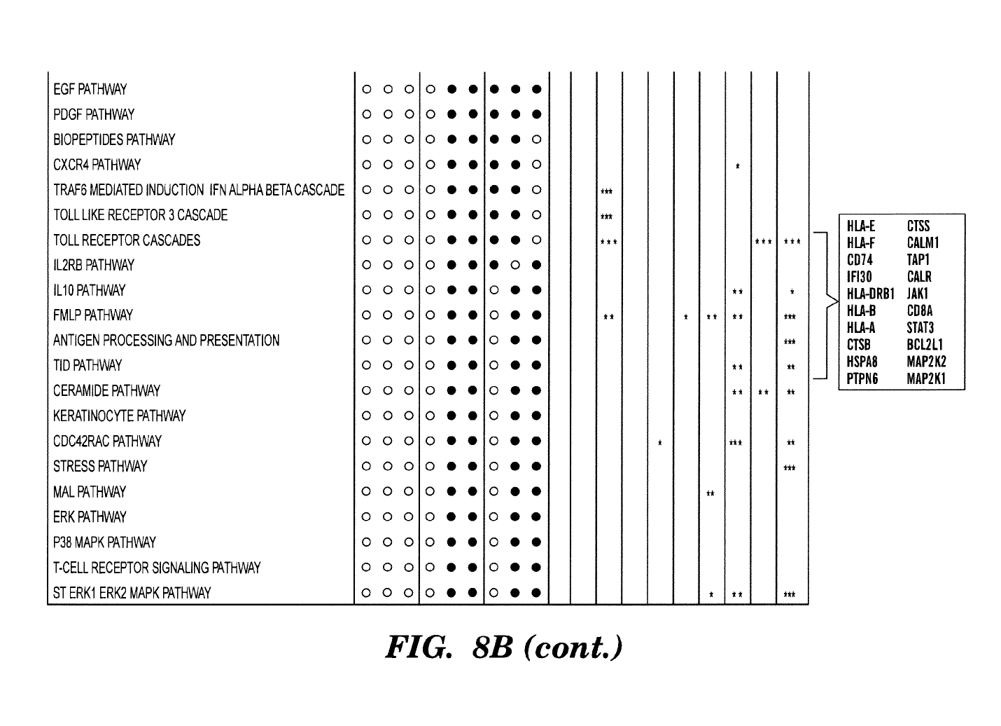

The Progressive I Upregulated pattern consisted of gene sets that were significantly enriched in both N2 and M subjects as compared to N1 subjects and with greater enrichments in M subjects at each time point (i.e., more time points with significant enrichments in M compared to N1 and N2) (FIG. 8A, solid dots indicating significant group differences). In addition to these across-group differences at each time point, most gene sets also showed significant changes across time points, particularly in the M group (FIG. 8A, asterisks indicating significant time difference). For example, gene sets for the cytochrome P450 (CYP450) family, steroid hormones, retinol metabolism, and cell adhesion pathways were upregulated in M and N2 with greater enrichment in M, based on both across- and within-group comparisons. The CYP450 enzyme family is involved in the oxidative metabolism of a variety of compounds to regulate the production of reactive oxygen species that, in turn, regulate oxidative stress as well as many other signaling pathways and cellular functions (29).

Gene sets linked to energy metabolism (electron transport chain, integration of energy metabolism) and insulin secretion pathways were also upregulated in M compared to N1 and N2, as indicated by the significant group differences at T0 and T1 and the relatively higher gene expression values seen. Although there was a slight downregulation in gene expression across the three time points in M, the gene expression values remained higher in the M group than the N1 and N2 groups.

Similarly, GSEA identified Progressive Downregulated gene sets with Progressive I pattern based on both across and within group comparisons. These gene sets are linked to inflammatory processes (NF-κB, TNF R2, CCR5, IL-7, RELA) and T cell signaling pathways (FIG. 8B). The expression patterns observed clearly show the progressive downregulation across N1, N2 and M, as well as across time points in M (FIG. 8B).

GSEA also identified pathways that had similar enrichments for M and N2 as compared to N1 where there was no significant difference between M and N2 at T0 and T1 (FIG. 8A, Progressive II). Only the M group, however, showed enrichment across time points in most of these pathways. These pathways were classified as Progressive II gene sets, for which both short-term and long-term practitioners (as compared to novices) had rapid enrichment within one session of RR practice. Progressive II Upregulated gene sets included pathways linked to glucose transport, neuroactive ligand receptor interaction and olfactory signaling, whereas downregulated gene sets were linked to immune response (CCR5, MEF2D, Phosphorylation of CD3 and TCR zeta chains, NTHI pathways) and mRNA preprocessing (maturation, metabolism, splicing and deadenylation) (FIG. 8B, Progressive II).

### Immune Response and Telomere Maintenance Related Pathways are Affected Among Long-Term RR Practitioners

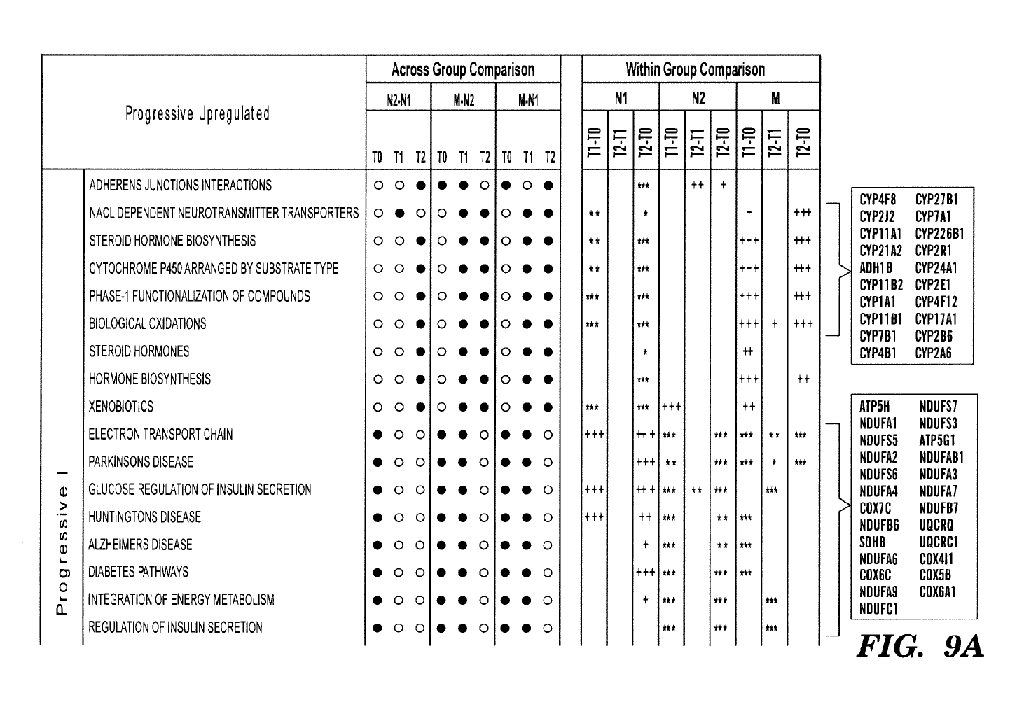

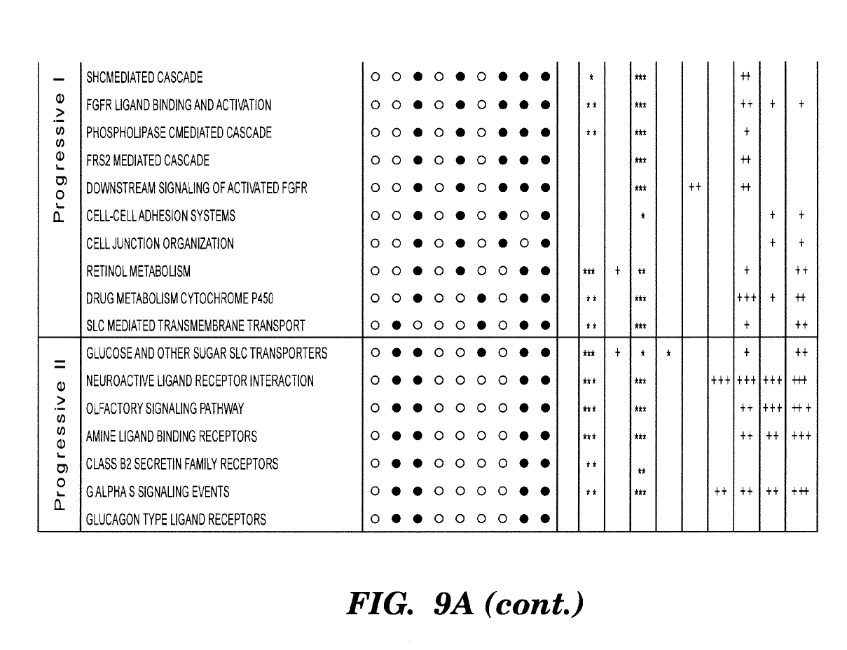

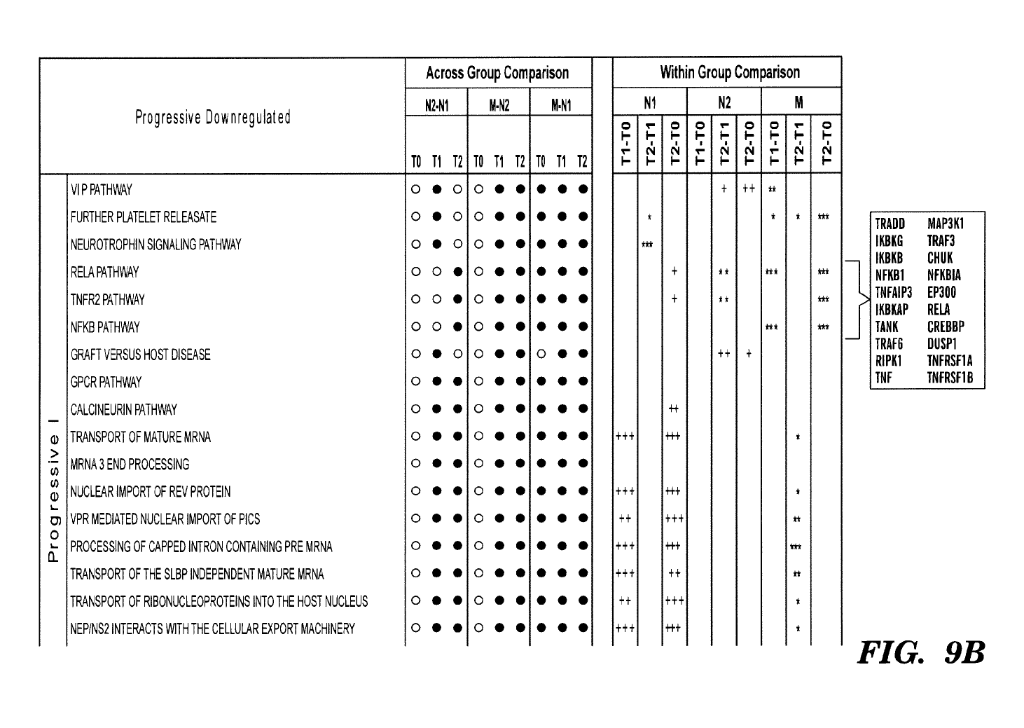

GSEA identified pathways that were significantly upregulated in M subjects at 2 or 3 time points compared to both N1 and N2, for which there were no significant group differences. Some of these pathways, however, even though they were elevated in the M group as compared to N1 and N2, were downregulated within the M group from T0 to T2 (FIG. 9A). These pathways, classified as Long-term Upregulated pathways, were linked to telomere maintenance and cardiac muscle contraction (FIG. 9A). Likewise, GSEA detected several pathways that were significantly downregulated in the M group as compared to N1 or N2 groups at 2 or 3 time points. In addition, the M group showed a significant downregulation in gene expression from T0 to T2 (FIG. 9B). These pathways were classified as Long-term Downregulated pattern, and were significantly associated with immune response (antigen processing and presentation, TOLL receptor cascade, CXCR4, CCR3, IL6, CD40, TLR3, B cell receptor signaling, IL10 and IL2RB signaling, FC gamma mediated phagocytosis), cell cycle (apoptotic pathways) as well as stress-related pathways (stress pathway, P38 MAPK) (FIG. 9B). Indeed, the downregulation of immune and inflammatory response pathways and upregulation of energy production pathways were consistent findings from the data using multiple different analytic approaches.

Upregulated Progressive Changes Induced by RR are Linked to Energy Production in Mitochondria: Systems Biology Analysis

To identify the key molecules—so-called focus gene hubs—affected by RR elicitation, systems biology analysis was applied to generate interactive gene networks. The interactive networks were generated from enriched genes of gene sets that are associated with RR practices identified by GSEA as described above. The networks were generated mainly on the basis of direct physical or biochemical protein-protein interactions, with a relatively small number of experimentally verified protein-DNA or protein-RNA interactions. The interaction information about the genes was obtained from public interaction databases or ingenuity commercial package (30-33). These interactive networks were further analyzed to identify network hubs using the bottleneck algorithm, which may represent the key nodes in the network.

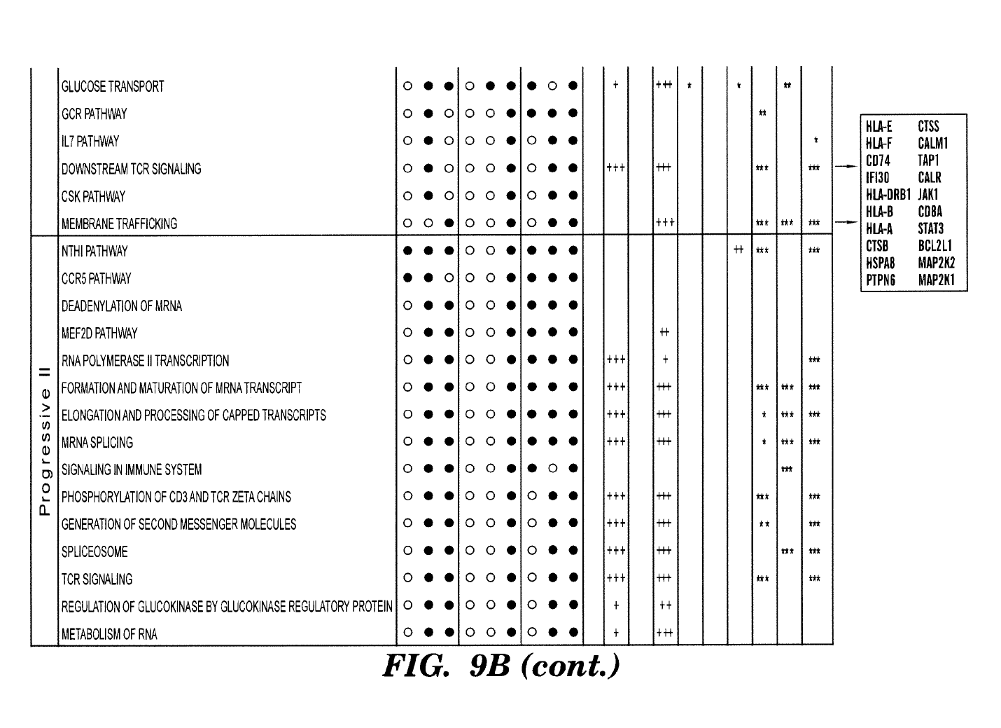

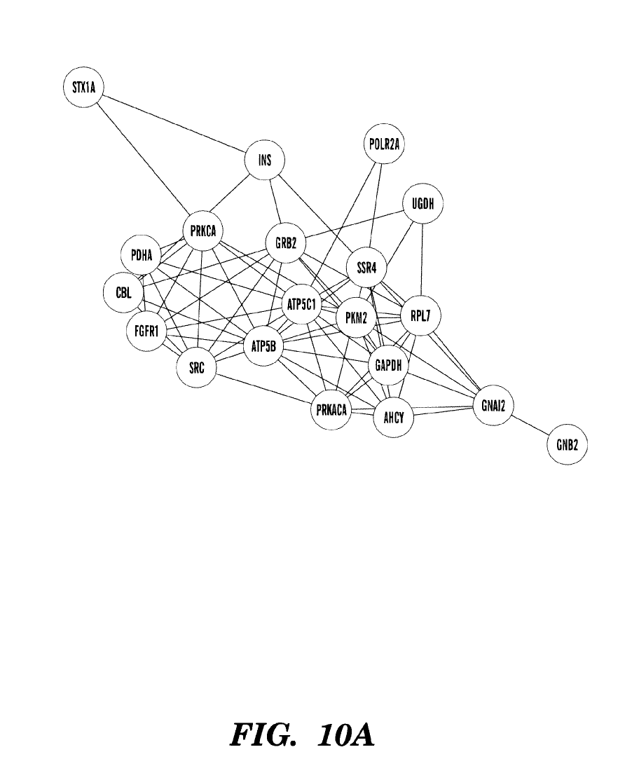

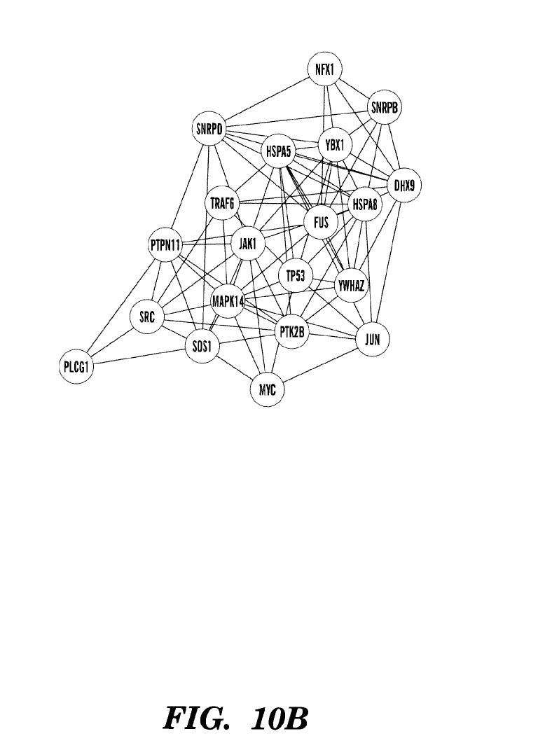

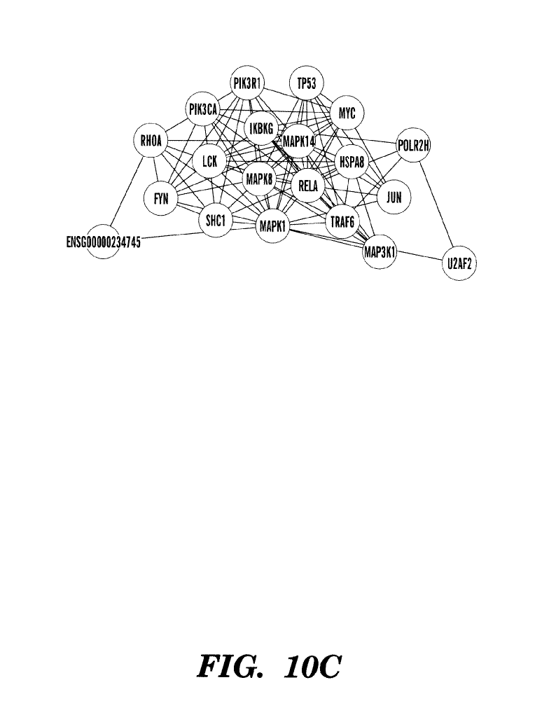

The analysis on 27 upregulated pathways with the Progressive I pattern (FIG. 8A) generated a complex network that consists of genes from pathways related to energy production (e.g., electron transport chain, integration of energy metabolism), metabolism (e.g., Retinol Metabolism, Hormone Biosynthesis), growth factors (i.e., FGFR ligand binding and activation, FRS2 mediated cascade) and glucose regulation (i.e., regulation of insulin secretion). Within this complex interactive network, the top 20 bottleneck genes (focus hubs) with the highest number of molecular interactions with neighboring molecules were identified. These focus hubs included the ATP synthase subunit gamma (ATP5C1), cAMP-dependent protein kinase (PRKACA) and insulin (INS) genes (FIG. 10A), all of which are linked to energy production and usage in mitochondria as well as glucose regulation (34). A list of the 20 bottleneck genes identified is: STX1A, POLR2A, INS, SSR4, PRKCA, PKM2, PDHA1, PRKACA, CBL, UGDH, FGFR1, RPL7, SRC, GAPDH, ATP5B, AHCY, ATP5C1, GNAI2, GRB2, GNB2.

### Upregulated Long-Term Changes Induced by RR are Linked to Telomerase Stability and Maintenance: Systems Biology Analysis

The interactive network and focus hub identification analysis on 14 Long-term Upregulated pathways identified an interactive network and also focus hubs. The focus gene hubs were identified using the bottleneck algorithm for identificasiton of the most interactive molecuels with a tree linke toplogical structure. The bottleneck algorithm ranks genes on the basis of significance level with smaller rank indicating increasing confidence. Genes linked to DNA stability, recombination and repair (i.e., HIST1H2BC, CACNA1C, and CYC1) as the top focus genes. These genes play a critical role in telomere stability and maintenance. The 20 genes identified were: CYC2, GNB2, GNB1, GNG12, F2, IL8, CACNA1C, POU2F1, PARD3, POLR2K, RARD6A, BRF1, INADL, RUVBL1, HIST1H2BC, H2AFX, PLG, PCNA, CXCL2, and RPA1.

### Progressive and Long-Term Downregulated Gene Expression Changes Induced by RR are Linked to Alteration of NF-κB-Dependent Pathways: Systems Biology Analysis

Interactive network analysis on the 23 downregulated pathways with Progressive I pattern generated a complex network that consists of genes from pathways related to inflammation (i.e., NF-κB, TNFR2, RELA), immune response (i.e., IL7, CCR5), T cell signaling (i.e., downstream TCR signaling) and mRNA processing. Within this interactive network we identified the top 20 focus hubs of these pathways to be related to NF-κB activity including MAPK14, MYC, PTPKB2, TP53, and TRAF6 (FIG. 10B). The 20 genes identified were: NFX1, PTPN11, SNRPD2, SRC, HSPA5, PLCG1, YBX1, SOS1, SNRPB, MAPK14, TP53, TRAF6, YWHAZ, FUS, PTK2B, HSPA8, JAK1, JUN, and MYC.

The interactive network analysis on the 15 downregulated pathways with acute Progressive II pattern revealed a similar enrichment of NF-κB activity related molecules (e.g., RELA, TRAF6, MAPK14, MAPK11, TP53, MYC). The interactive network and focus hub identification analysis was performed on genes from 15 Progressively Down-regulated (Progressive I) pathways linked to mRNA processing and immune response. The focus gene hubs were identified using the bottleneck algorithm for identification of the most interactive molecules with a tree like topological structure. The bottleneck algorithm ranks genes on the basis of significance level with smaller rank indicating increasing confidence. Bottleneck ranks were assigned from 1 to 20.

The interactive network analysis on the Long-term Downregulated pathways also revealed the enrichment of NF-κB activity related molecules (e.g., MAPK1, MAPK3, JUN, SRC, TRAF6). The 20 genes identified were: ENSGOOOOO234745, EGFR, TRAF6, MAP3K1, RAC1, YWHAZ, PRKACA, TP53, SRC, MYC, CBL, JUN, STAT1, UBB, PIK3CA, HSPA5, MAPK14, CSNK2A1, MAPK3, and SP1.

Finally, in an attempt to better understand the molecular mechanism of RR and to identify the most critical focus genes, we merged the Long-term and Progressive systems biology networks were merged and the focus hubs investigated in this integrated network. The network of the top 20 focus hubs of this analysis clearly showed enrichment for NF-κB upstream and downstream target molecules (e.g., RELA, IKBKG, TRAF6, MAPK14, MAPK11, TP53, MYC) (FIG. 6C) and identified NF-κB associated molecules (e.g., MAPK14, HSPA5, PTK2B) as top focus hub genes, indicating the critical role of NF-κB in RR. These findings further support the notion that reduced NF-κB activity may be associated with RR elicitation. The 20 genes identified were: PIK3R1, RHOA, TP53, LCK, MYC, MAPK8, PIK3CA, MAPK1, IKBKG, TRAF6, MAPK14, FYN, HSPA8, SHC1, POLR2H, MAPK31, JUN, U2AF2, RELA, and NSGOOOOO234745.

### Discussion

Substantial research on mind-body interventions has established their ability to reduce chronic stress and enhance wellness through induction of the RR (2-4, 7, 14, 17, 18, 23); however, little is known about the molecular mechanisms underlying RR-induced physiological changes. Previously, the first evidence was provided that RR practice results in specific, lasting base-level gene expression changes that are opposite to transcriptional changes induced by chronic stress (22). The study indicated that distinctive gene expression patterns associated with long- and short-term RR practices are sustained outside of RR-elicitation sessions. In contrast to the previous study, in the present study, the rapid and transient transcriptome changes (i.e., ‘temporal’ changes) were investigated during one session of RR practice among practitioners with years of practice (M) and novices before (N1) and after (N2) 8 weeks of RR training. It was reasoned that temporal expression analysis across several time points would enable the identification of the immediate effects of one session of RR on gene expression and signaling and that these effects would differ among N1, N2 and M groups. Temporal analysis enables identification of genes that are affected by RR at multiple time points and reduces the likelihood of identifying false positives.

Analysis of the transcriptome data revealed that temporal modulation of gene expression occurred in both short-(N2) and long-term (M) practitioners as compared to novices (N1). Long-term RR practitioners exhibited more pronounced and consistent immediate gene expression changes as compared to short-term practitioners. Some genes were modified only in long-term practitioners (Long-term patterns), whereas others were modified in both short- and long-term practitioners with a greater intensity in the latter (Progressive patterns). The Progressive pattern indicates the cumulative effects of RR practices on gene expression alteration and provides a genomics rationale for the recommendation to practice consistently in order to achieve the downstream health benefits of RR-based mind body approaches.

Importantly, this study demonstrates that one session of RR practice induces rapid changes in gene expression (on the order of minutes) that are linked to a select set of biological pathways among both long-term and short-term practitioners that might explain the health benefits of RR practices. These gene have been linked to pathways responsible for energy metabolism, electron transport chain, biological oxidation and insulin secretion. These pathways play central roles in mitochondrial energy mechanics, oxidative phosphorylation and cell aging (34, 35). It was hypothesized that upregulation of biological oxidation gene sets may enhance efficiency of oxidation-reduction reactions and thereby reduce oxidative stress.

The GSEA findings are further supported by the results from our systems biology analysis, which identified insulin (INS) and ATP synthase subunit gamma (ATP5C1) as top focus hubs. The mitochondrial ATP synthase is critical in regulating the production of adenosine triphosphate (ATP), which in turn is a key determinant for secretion of insulin from 13-cells in response to glucose. Mutations in ATP synthase leading to its impaired signaling have been shown to induce oxidative stress and impaired insulin secretion in 13-cells (35). Thus, RR practice may enhance mitochondrial energy producing pathways and modulate downward stress-related pathways that culminate in cell aging and apoptosis. By upregulating ATP synthase—with its central role in mitochondrial energy mechanics, and oxidative phosphorylation and cell aging—RR may act to buffer against cellular overactivation with overexpenditure of mitochondrial energy that results in excess reactive oxygen species production (36). We thus postulate that upregulation of the ATP synthase pathway may play an important role in translating the beneficial effects of the RR.

Gene sets identified by GSEA as progressively downregulated by RR practices are linked to pathways that play critical roles in the inflammatory response, including those connected with the pro-inflammatory transcription factors NF-κB and RELA, and TNFR2, IL7 and TCR signaling. Systems biology analysis supported these findings by identifying NF-κB associated molecules (e.g. MAPK14, HSPA5, PTK2B) as top focus hub genes. Downregulation of NF-κB inflammatory response gene sets may lead to reductions in oxidative stress, insulin resistance and apoptosis (37). NF-κB has been identified as a potential bridge between psychosocial stress and oxidative cellular activation (38). Induction of NF-κB in PBMCs was observed in 17 of 19 volunteers upon psychosocial stress exposure, correlating with elevated catecholamine and cortisol levels. Likewise, the stress of awaiting breast biopsy has been found to activate NF-κB in women (39). Furthermore, enhanced expression of stress-mediating MAPK14 was detected in PBMCs from graduate students under psychological stress (40). In a vicious cycle, psychosocial stress can cause chronic mitochondrial oxidative stress that can lead to the metabolic syndrome (hypertension, obesity, insulin resistant diabetes mellitus, and hyperlipidemia) (41, 42). This stress can lead to activation of NF-κB, which in turn can worsen oxidative stress and the metabolic syndrome.

Furthermore, NF-κB activation in PBMCs correlates with peripheral levels of oxidative stress and can be reduced by therapeutic interventions that decrease oxidative stress (43). Therefore, our finding that RR elicitation is associated with downregulation of the NF-κB node and its associated gene sets might be a key factor for explaining the clinical benefits of RR elicitation and provides a method for understanding the molecular mechanisms underlying the health benefits of RR through stress reduction.

Long-term RR practice, moreover, upregulated pathways associated with genomic stability such as telomere packing, telomere maintenance and tight junction interaction. Telomere dysfunction can cause disruption of mitochondrial regulators and cause mitochondrial compromise that ends in apoptosis (44). Increased telomerase activity is also considered to protect against cancer and other diseases. Findings of several recent studies support the notion that mind/body interventions such as RR may enhance telomerase pathways. 3 month of meditation in 30 participants resulted in increased immune cell telomerase activity when compared to 30 matched control subjects (45). Likewise, Practitioners of Sudarshan Kriya which is one form of RR demonstrated enhanced telomerase activity (46). In contrast, psychological stress has been linked to reduced telomerase activity, shortening of telomeres, and cell aging (49, 50). Telomere length has been linked to insulin resistance and the findings of insulin signaling as a key target that is upregulated progressively as the time of RR practice increases corroborates this association (51).

Systems biology analysis identified histone (HIST1H2BC), calcium channel (CACNA1C) and cytochrome C (CYC1) among top focus hubs of the Long-term Upregulated pathways. HIST1H2BC is a core component of the nucleosome and is thereby essential to transcriptional regulation, DNA repair, DNA replication and chromosomal stability. Cytochrome C is an important member of the mitochondrial respiratory and energy production complex that again may provide an insight into the role of RR in mitochondrial energy efficiency. CACNA1C, a calcium channel gene, mediates the entry of calcium ions into excitable cells and is also involved in a variety of calcium-dependent processes, including muscle contraction, hormone and neurotransmitter release, gene expression, cell motility, cell division and cell death. Therefore, the upregulation of HIST1H2BC, CACNA1C, and CYC1 that was associated with long-term RR practice might play an important role in translating the beneficial effects of RR.

Similarly, pathway enrichment and systems biology analysis on long-term RR downregulated genes revealed associations with pathways involved in immune response (e.g. IL6, IL10, CCR3, antigen processing and presentation, TCR signaling), apoptosis (e.g. Apoptosis, Ceramide, PML) and stress response (e.g. stress pathway, MTOR). Psychological effects on PBMC gene expression associated with DNA repair mechanisms and immune response have been observed in women with postpartum depression, thus linking psychological stress to deregulated immune function and DNA repair that could be impacted by RR (52). A most recent study, furthermore, links social environmental stress in rhesus macaques to distinct effects on immune system gene expression in PBMCs that enables to predict social status solely based on gene expression (53). Interestingly, similar immune response pathways such as T cell activation and chemokine signaling were modulated both by RR in the study and by social dominance in the rhesus macaques. These results demonstrate the possible multi-level effects of RR in modulating immune and stress responses that counter stress-induced transcriptome changes.

In summary, the first study to employ advanced genomic analysis methodology and systems biology analysis was conducted to examine temporal transcriptional changes during one session of RR practice and found that RR practice induced upregulation of ATPase and insulin function. This suggests that RR may enhance mitochondrial energy production and utilization. At the same time RR induced downregulation of NF-κB-dependent pathways, with effects on upstream and downstream targets that may mitigate oxidative stress. These findings, while preliminary, suggest that RR practice, by promoting what might be called mitochondrial resiliency, may be important at the cellular level for the downstream health benefits associated with reducing psychosocial stress. These findings provide a framework for further deciphering the in-depth molecular pathways associated with the clinical benefits of the RR. This molecular mechanism of RR will be confirmed using secondary biochemical testing.

### REFERENCES

### Example 2

1. H. Benson, J. F. Beary, M. P. Carol, The relaxation response. Psychiatry 37, 37 (February, 1974).
2. H. Benson, The Relaxation Response. (HarperCollins, 1975).
3. J. A. Dusek, H. Benson, Mind-body medicine: a model of the comparative clinical impact of the acute stress and relaxation responses. Minn Med 92, 47 (May, 2009).
4. S. Cohen, D. Janicki-Deverts, G. E. Miller, Psychological stress and disease. JAMA: the journal of the American Medical Association 298, 1685 (Oct. 10, 2007).
5. J. A. Dusek et al., Stress management versus lifestyle modification on systolic hypertension and medication elimination: a randomized trial. Journal of alternative and complementary medicine 14, 129 (March, 2008).
6. M. Nakao et al., Anxiety is a good indicator for somatic symptom reduction through behavioral medicine intervention in a mind/body medicine clinic. Psychother Psychosom 70, 50 (January-February, 2001).
7. H. Benson et al., Treatment of anxiety: a comparison of the usefulness of self-hypnosis and a meditational relaxation technique. An overview. Psychother Psychosom 30, 229 (1978).
8. G. D. Jacobs et al., Multifactor behavioral treatment of chronic sleep-onset insomnia using stimulus control and the relaxation response. A preliminary study. Behav Modif 17, 498 (October, 1993).
9. C. M. Morin, B. Gaulier, T. Barry, R. A. Kowatch, Patients' acceptance of psychological and pharmacological therapies for insomnia. Sleep 15, 302 (August, 1992).
10. S. V. Hegde et al., Effect of 3-month yoga on oxidative stress in type 2 diabetes with or without complications: a controlled clinical trial. Diabetes Care 34, 2208 (October, 2011).
11. J. A. Astin, W. Beckner, K. Soeken, M. C. Hochberg, B. Berman, Psychological interventions for rheumatoid arthritis: a meta-analysis of randomized controlled trials. Arthritis Rheum 47, 291 (Jun. 15, 2002).
12. J. A. Galvin, H. Benson, G. R. Deckro, G. L. Fricchione, J. A. Dusek, The relaxation response: reducing stress and improving cognition in healthy aging adults. Complement Ther Clin Pract 12, 186 (August, 2006).
13. C. N. Alexander, E. J. Langer, R. I. Newman, H. M. Chandler, J. L. Davies, Transcendental meditation, mindfulness, and longevity: an experimental study with the elderly. J Pers Soc Psychol 57, 950 (December, 1989).
14. H. Benson, Proctor, W., Relaxation Revolution. (Simon & Schuster, 2011).
15. J. A. Dusek et al., Association between oxygen consumption and nitric oxide production during the relaxation response. Med Sci Monit 12, CR1 (January, 2006).
16. R. K. Wallace, H. Benson, A. F. Wilson, A wakeful hypometabolic physiologic state. Am J Physiol 221, 795 (September, 1971).
17. H. B. Benson, John F.; Carol, Mark P., The relaxation response. Psychiatry: Journal for the Study of Interpersonal Processes, 37, 37 (1974).
18. D. W. Hoffman, S. K. Salzman, M. Marchi, E. Giacobini, Norepinephrine levels in peripheral nerve terminals during development and aging. J Neurochem 34, 1785 (June, 1980).
19. C. K. Peng et al., Heart rate dynamics during three forms of meditation. Int J Cardiol 95, 19 (May, 2004).
20. S. W. Lazar et al., Functional brain mapping of the relaxation response and meditation. NeuroReport 11, 1581 (May 15, 2000).
21. G. D. Jacobs, H. Benson, R. Friedman, Perceived benefits in a behavioral-medicine insomnia program: a clinical report. Am J Med 100, 212 (February, 1996).
22. J. A. Dusek et al., Genomic counter-stress changes induced by the relaxation response. PLoS One 3, e2576 (2008).
23. B. H. Chang, J. A. Dusek, H. Benson, Psychobiological changes from relaxation response elicitation: long-term practitioners vs. novices. Psychosomatics 52, 550 (November-December, 2011).
24. A. Subramanian, H. Kuehn, J. Gould, P. Tamayo, J. P. Mesirov, GSEA-P: a desktop application for Gene Set Enrichment Analysis. Bioinformatics 23, 3251 (Dec. 1, 2007).
25. A. Subramanian et al., Gene set enrichment analysis: a knowledge-based approach for interpreting genome-wide expression profiles. Proc Natl Acad Sci USA 102, 15545 (Oct. 25, 2005).
26. P. Tamayo et al., Interpreting patterns of gene expression with self-organizing maps: methods and application to hematopoietic differentiation. Proc Natl Acad Sci USA 96, 2907 (Mar. 16, 1999).
27. C. L. Wilson, C. J. Miller, Simpleaffy: a BioConductor package for Affymetrix Quality Control and data analysis. Bioinformatics 21, 3683 (Sep. 15, 2005).
28. W. Huang da et al., DAVID Bioinformatics Resources: expanded annotation database and novel algorithms to better extract biology from large gene lists. Nucleic Acids Res 35, W169 (July, 2007).
29. R. C. Zangar, D. R. Davydov, S. Verma, Mechanisms that regulate production of reactive oxygen species by cytochrome P450. Toxicol Appl Pharmacol 199, 316 (Sep. 15, 2004).
30. M. Jayapandian et al., Michigan Molecular Interactions (MiMI): putting the jigsaw puzzle together. Nucleic Acids Res 35, D566 (January, 2007).
31. T. S. Keshava Prasad et al., Human Protein Reference Database—2009 update. Nucleic Acids Res 37, D767 (January, 2009).
32. S. Mostafavi, D. Ray, D. Warde-Farley, C. Grouios, Q. Morris, GeneMANIA: a real-time multiple association network integration algorithm for predicting gene function. Genome Biol 9 Suppl 1, S4 (2008).
33. I. Xenarios et al., DIP: The Database of Interacting Proteins: 2001 update. Nucleic Acids Res 29, 239 (Jan. 1, 2001).
34. A. Wiederkehr, C. B. Wollheim, Minireview: implication of mitochondria in insulin secretion and action. Endocrinology 147, 2643 (June, 2006).
35. M. C. Saleh et al., Mutated ATP synthase induces oxidative stress and impaired insulin secretion in beta-cells of female BHE/cdb rats. Diabetes Metab Res Rev 24, 392 (July-August, 2008).
36. S. Zou, S. Meadows, L. Sharp, L. Y. Jan, Y. N. Jan, Genome-wide study of aging and oxidative stress response in Drosophila melanogaster. Proc Natl Acad Sci USA 97, 13726 (Dec. 5, 2000).
37. Y. Hasegawa et al., Blockade of the Nuclear Factor-kappaB Pathway in the Endothelium Prevents Insulin Resistance and Prolongs Life Spans. Circulation 125, 1122 (Mar. 6, 2012).
38. A. Bierhaus, P. M. Humpert, P. P. Nawroth, NF-kappaB as a molecular link between psychosocial stress and organ dysfunction. Pediatric nephrology 19, 1189 (November, 2004).
39. M. Nagabhushan, H. L. Mathews, L. Witek-Janusek, Aberrant nuclear expression of AP-1 and NFkappaB in lymphocytes of women stressed by the experience of breast biopsy. Brain, behavior, and immunity 15, 78 (March, 2001).
40. K. Morita et al., Expression analysis of psychological stress-associated genes in peripheral blood leukocytes. Neurosci Lett 381, 57 (Jun. 10-17, 2005).
41. N. G. Abraham, E. J. Brunner, J. W. Eriksson, R. P. Robertson, Metabolic syndrome: psychosocial, neuroendocrine, and classical risk factors in type 2 diabetes. Ann N Y Acad Sci 1113, 256 (October, 2007).
42. K. L. Tamashiro, Metabolic syndrome: links to social stress and socioeconomic status. Ann N Y Acad Sci 1231, 46 (August, 2011).
43. M. A. Hofmann et al., Peripheral blood mononuclear cells isolated from patients with diabetic nephropathy show increased activation of the oxidative-stress sensitive transcription factor NF-kappaB. Diabetologia 42, 222 (February, 1999).
44. E. Sahin et al., Telomere dysfunction induces metabolic and mitochondrial compromise. Nature 470, 359 (Feb. 17, 2011).
45. T. L. Jacobs et al., Intensive meditation training, immune cell telomerase activity, and psychological mediators. Psychoneuroendocrinology 36, 664 (June, 2011).
46. H. Sharma et al., Gene expression profiling in practitioners of Sudarshan Kriya. J Psychosom Res 64, 213 (February, 2008).
47. D. Ornish et al., Increased telomerase activity and comprehensive lifestyle changes: a pilot study. Lancet Oncol 9, 1048 (November, 2008).
48. D. Ornish et al., Changes in prostate gene expression in men undergoing an intensive nutrition and lifestyle intervention. Proc Natl Acad Sci USA 105, 8369 (Jun. 17, 2008).
49. E. Epel, J. Daubenmier, J. T. Moskowitz, S. Folkman, E. Blackburn, Can meditation slow rate of cellular aging? Cognitive stress, mindfulness, and telomeres. Ann N Y Acad Sci 1172, 34 (August, 2009).
50. E. S. Epel et al., Cell aging in relation to stress arousal and cardiovascular disease risk factors. Psychoneuroendocrinology 31, 277 (April, 2006).
51. A. Adaikalakoteswari, M. Balasubramanyam, R. Ravikumar, R. Deepa, V. Mohan, Association of telomere shortening with impaired glucose tolerance and diabetic macroangiopathy. Atherosclerosis 195, 83 (November, 2007).
52. R. H. Segman et al., Blood mononuclear cell gene expression signature of postpartum depression. Mol Psychiatry 15, 93 (January, 2010).
53. J. Tung et al., Social environment is associated with gene regulatory variation in the rhesus macaque immune system. Proc Natl Acad Sci USA 109, 6490 (Apr. 24, 2012).
54. C. Li, W. H. Wong, Model-based analysis of oligonucleotide arrays: expression index computation and outlier detection. Proc Natl Acad Sci USA 98, 31 (Jan. 2, 2001).
55. R. Fisher, On the Interpretation of x2 from Contingency Tables, and the Calculation of P. Journal of the Royal Statistical Society 85, 7 (1922).
56. D. Merico, R. Isserlin, O. Stueker, A. Emili, G. D. Bader, Enrichment map: a network-based method for gene-set enrichment visualization and interpretation. PLoS One 5, e13984 (2010).
57. P. Shannon et al., Cytoscape: a software environment for integrated models of biomolecular interaction networks. Genome Res 13, 2498 (November, 2003).
58. G. R. Mishra et al., Human protein reference database—2006 update. Nucleic Acids Res 34, D411 (Jan. 1, 2006).
59. T. S. Prasad, K. Kandasamy, A. Pandey, Human Protein Reference Database and Human Proteinpedia as discovery tools for systems biology. Methods Mol Biol 577, 67 (2009).
60. C. Y. Lin et al., Hubba: hub objects analyzer—a framework of interactome hubs identification for network biology. Nucleic Acids Res 36, W438 (Jul. 1, 2008).

### Example 3

### Impact of a GI Mind Body Intervention (MBI) Upon Mixed Group of Patients with Irritable Bowel Syndrome and Inflammatory Bowel Disease

Despite different pathophysiologies between Irritable Bowel Syndrome (IBS) and Inflammatory Bowel Disease (IBD), many patients suffer similar abdominal complaints with an impact on quality of life from chronic illness. Delivery of Mind Body Interventions (MBI) has been proven effective for some chronic illnesses; however, the impact of the outcome on a mixed patient group of IBS and IBD patients has not been examined. The impact of MBI on quality of life and inflammatory markers in patients with IBS and IBD was examined.

### Methods

This pilot prospective single center study utilizes MBI for the treatment of IBS and IBD using cognitive behavioral therapy, relaxation response and meditation techniques, as established by the Benson-Henry Institute for Mind Body Medicine, discussed above in Example 2. Patients with either IBS or IBD were enrolled prospectively as one cohort in a 10-week group program. Baseline, Week 5, 10 (effect from active intervention), and 13 (short term persistent effect after intervention) assessments with validated disease specific instruments, IBD Questionnaire (IBD-Q) and IBS Quality of Life (IBS-QOL), were used to assess disease specific impact. The Brief Pain Inventory (BPI) severity and interference, Pain Catastrophizing Scale (PCS), State Trait Anxiety Inventory (STAI) were given to both groups to evaluate generalized impact of the intervention in the group as a whole along with ESR and CRP.

### Results

19 IBS (Mean age 47 yrs, 15 F) and 29 IBD (Mean age 40 yrs, 17 F) were enrolled with 6 IBD/41BS dropout during the program. IBS-QOL score decreased suggesting clinical improvement from 34 at baseline to 26 at week 10 and 18 at week 13. (p=0.01). IBD-Q scores increased also suggesting clinical improvement from 170 to 184 at week 10 and remained at 184 at week 13 (p=0.01) BPI measures did not change in either group (p=0.43). PCS improved overall on average across both disease groups, but for 0 to 10 wks (p<0.001), the improvement was much greater for IBD patients. STAI state also improved overall, although here the effect was larger among IBS patients (p<0.001) Both groups improved for STAI trait in a similar amount (p<0.001). ESR and CRP did not change significantly and none of the measurements correlated with ESR/CRP changes.

### CONCLUSION

This pilot study demonstrated that a generalized GI MBI can make a clinical impact on a mixed group of IBS and IBD patients improving disease specific measures. Changes were seen in pain catastrophizing and anxiety in both groups. However, the results suggest effects that are independent of the gross inflammatory process.

### Example 4

### Genomic Expression Changes Underlying Mind-Body Practices

Genomic markers that reflect responsiveness to a relaxation response (RR) intervention have been detected in healthy volunteers, but have not been examined in clinical conditions for which RR is effective (e.g., hypertension). This study aimed to explore the impact of an RR intervention on gene expression in hypertensive individuals.

### Methods

Transcriptional profiling analyses was performed on the Peripheral Blood Mononuclear Cells (PBMCs) obtained before (Pre RR) and after (Post RR) 8 weeks of intervention from a pilot group of 4 hypertensives, by the methods discussed above in Example 2. Transcriptional profiling used Affymetrix HT HG-U133+ PM arrays, containing >47,000 transcripts corresponding to 38,500 genes. The arrays were normalized using the robust multi-chip analysis (RMA) algorithm. After normalization and preprocessing, differentially expressed genes were identified using the random variance model based t-test. To understand the underlying biological mechanisms associated with RR regulated genes, Gene ontology (GO), pathways and geneset enrichment analysis (GSEA) were performed, by the methods discussed above in Example 2.

### Results

A total of 474 transcripts were significantly differentially expressed (p<0.05) between the Pre and Post RR conditions. Hierarchical clustering of the top differentially expressed genes demonstrates striking segregation between Pre and Post RR conditions. The GO analysis identified significantly affected categories (p<0.05) that include “mRNA metabolic process,” “positive regulation of RNA metabolic process,” “Calcium signaling pathway” and “programmed cell death.” The pathway analysis identified the over-representation (p<0.05) of differentially expressed genes to “cell cycle G/M checkpoint regulation,” “P53 signaling,” “Inositol metabolism signaling,” “Apoptosis Signaling,” “MIF Regulation of Innate Immunity,” and “Cardiac β-adrenergic Signaling.” Furthermore, GSEA analysis identified upregulation of 263 genesets (p<0.05) in Post RR including phosphatidylinositol signaling system, IFN-γ endothelial up, B cell receptor signaling pathway, FAS pathway, VEGF signaling pathway, Cardiac EGF pathway and MAPK pathway.

### CONCLUSION

These results suggest specific biochemical pathways on which to expand future research in the study of mind/body therapies in clinical populations.

### Example 5

### Genomic Determinants of a Relaxation Response Resiliency Program in Inflammatory Bowel Disease and Irritable Bowel Syndrome

Stress has been implicated in exacerbations of inflammatory bowel diseases (IBD) and irritable bowel syndrome (IBS). The relaxation response (RR) is a physiological and psychological opposite to stress response and should potentially counteract stress-induced physiological changes. Genomic changes elicited by the RR are thought to counterregulate genes induced by stress and inflammation. The aim was of this study was to identify and compare genomic determinants of a relaxation response resiliency program (RRRP) in IBD and IBS using transcriptional profiling.

### Methods

The peripheral blood gene expression profile of 10 IBD and 9 IBS patients was assessed before and after a 10 week RRRP using HT U133+ PM arrayplates (Affymetrix), by the above-discussed methods (see Example 2). After normalization using the robust multi-chip analysis (RMA) algorithm, differentially expressed genes were identified using Student t-test. To understand the underlying biological mechanisms associated with RRRP regulated genes and the gene sets with concordant differences between the two groups, functional categories, canonical pathways, interactive network, and Geneset Enrichment Analysis (GSEA) was performed by the above-discussed methods (see Example 2).

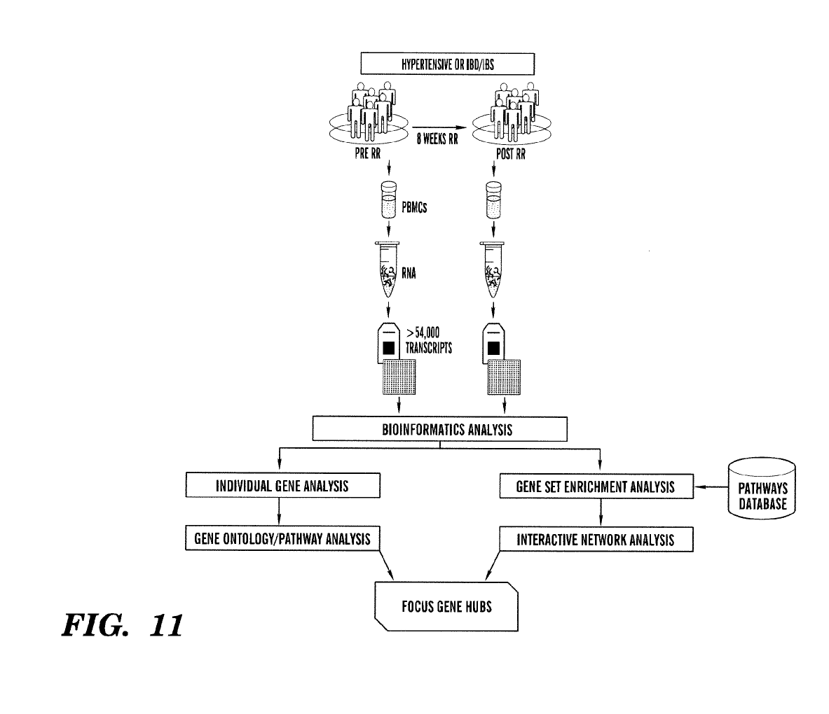

FIG. 11 shows a schematic view of this study design and analysis plans. The transcriptome profiling was performed on peripheral blood mononuclear cells (PBMCs) collected before and after 8 weeks weeks of RR. The global transcriptome of PBMCs was profiled using HT_U133 Plus PM arrays containing >54,000 transcripts. The transcriptome data were analyzed using high-level bioinformatics algorithms to identify differentially expressed transcripts, significantly affected pathways and systems biology networks that are related to RR elicitation.

### Results

IBS-QOL scores decreased (p=0.03), showing clinical improvement from 39.7 at baseline to 29.3 at week 10. IBD-Q scores increased (p=0.21), suggesting a trend towards clinical improvement from 163.6 at baseline to 185.8 at week 10. Principal component analysis of the baseline data separated IBD from most of the IBS patients. However, IBS samples appear to be more heterogeneous with some of them clustering together with IBD. At baseline, 392 genes (P value <0.01) significantly linked to immunological and inflammatory diseases with NF-κB and TGF-β as focus hubs differentiating IBS from IBD. Transcriptional profiling identified 512 differentially expressed transcripts for IBD and 280 for IBS (P value <0.01) between Pre and Post RRRP training. In IBD, GSEA identified significant (P<0.05, FDR<30%, 500 permutations) upregulation of genesets involved in ribosomal protein activity and serine type endopeptidase inhibitor activity and downregulation of FAS, TNFR1, Ill, apoptosis and TGF-13 pathways. Similarly in IBS, cell cycle regulation and chromosome segregation related genesets were significantly upregulated and serine metabolism and muscle contraction genesets were downregulated by RRRP. The regulatory hubs of the most comprehensive interactive network, meaning the genes with highest degrees of interactions, consisted of NF-κB, AKT, Insulin and tumor necrosis factor (TNF) for IBD and IBS, and TGFβ1 and PPARG in IBD only and BCL2, CSF1, and MAPK1 in IBS only.

### CONCLUSION

This data provide evidence that a RRRP in IBD and IBS patients elicits specific and consistent gene expression changes. NF-κB emerges as a common focus molecule that may counteract the harmful effects of stress in both IBS and IBD. Table 10, below lists genes that were identified as consistently and significantly changed in expression levels in subjects with IBD and IBS, following successful relaxation response practice. These genes are suitable for use in assaying for a RR in a subject diagnosed with or at risk for IBD and IBS.

TABLE 10
A preferred list of genes top focus genes identified from systems
biology analysis of genes that are significantly dysregulated after
8 weeks of RR practice in IBD/IBS patients.

| | | Directional |
|---|---|---|
| | | change (~1.2 |
| Gene Symbol | Description | fold) |
| NFKB | Nuclear Factor-KappaB | Down |
| MAPK1 | mitogen-activated protein kinase 1 | Down |
| NR3C1 | nuclear receptor subfamily 3, group C, | Down |
| | member 1 (glucocorticoid receptor) | |
| FOXO1 | forkhead box O1 | Down |
| PPARA | peroxisome proliferator-activated receptor | Down |
| | alpha | |
| EP300 | E1A binding protein p300 | Down |
| TNF | tumor necrosis factor | Down |
| TNFSF11 | tumor necrosis factor (ligand) superfamily, | Down |
| | member 11 | |
| JUN | jun oncogene | Down |
| ESR1 | Estrogen Receptor 1 | UP |
| SMAD1 | SMAD FAMILY MEMBER 1 | Down |
| GADD45A | growth arrest and DNA-damage-inducible, | Down |
| | alpha | |

### Example 6

### Genomics Determinants of Relaxation Response Responsiveness in Hypertensive Subjects

Essential hypertension affects about 35% of the adult US population and costs roughly $100 billion annually. When left untreated, it drastically increases the risks for stroke, cardiovascular disease and chronic renal failure. Various forms of stress management training, in particular eliciting the relaxation response (RR), have been demonstrated to reduce essential hypertension and may be an alternative or adjunct to antihypertensive drug therapy. Building upon all beneficial clinical effects of RR in stress management, this study was performed to identify genomic determinants of responsiveness to RR for controlling hypertension in uncontrolled, stage 1-hypertensive patients.

### Methods

Transcriptional profiling analyses was performed on the Peripheral Blood Mononuclear Cells (PBMCs) obtained before (Pre RR) and after (Post RR) 8 weeks of RR from a pilot group of 19 hypertensive, by the methods described above in Example 2. Transcriptional profiling used Affymetrix HT HG-U133+ PM arrays, containing >47,000 transcripts corresponding to 38,500 genes. The arrays were normalized using the robust multi-chip analysis (RMA) algorithm. After normalization and preprocessing, differentially expressed genes were identified using the random variance model based paired t-test. To understand the underlying biological mechanisms associated with RR regulated genes, Gene ontology (GO), pathways and systems biology analysis were performed by the methods described above in Example 2.

FIG. 11 shows a schematic view of this study design and analysis plans. The transcriptome profiling was performed on peripheral blood mononuclear cells (PBMCs) collected before and after 8 weeks weeks of RR. The global transcriptome of PBMCs was profiled using HT_U133 Plus PM arrays containing >54,000 transcripts. The transcriptome data were analyzed using high-level bioinformatics algorithms to identify differentially expressed transcripts, significantly affected pathways and systems biology networks that are related to RR elicitation.

### Results

The supervised analysis of Pre-RR and Post-RR transcriptome data from 19 hypertensive subjects identified 295 significantly differentially expressed genes (P value <0.01). The Gene Ontology enrichment analysis identified significantly affected categories (P value <0.05) that include “TGFβ signaling pathway”, “regulation of blood pressure”, “regulation of metabolic processes” and “cell motion and migration”. The pathways analysis identified the over-representation (P<0.05) of RR differentially expressed genes to “Mitochondrial Dysfunction”, “Oxidative Phosphorylation”, “Integrin signaling”, “Insulin receptor signaling”, “Inositol metabolism signaling” and “Cardiac β-adrenergic Signaling”. Interactive network analyses of RR-affected genes identified many a cardiovascular systems and metabolism related network with insulin receptor as one of the critical molecules (focus hubs). Additionally, the interactive network analysis also identified an immune response and inflammation related network with NF-kB and its upstream and downstream targets (e.g. PI3K, AKT, MAPK) as focus hub. These focus molecules depict overlap with system biology modules established by our group to depict beneficial effects of RR in healthy subjects.

### CONCLUSION

The data provide first evidence that RR in Hypertension patients elicits specific genomics determinants. Insulin (INS) and NF-κB surface as common focus molecules that may counteract the harmful effects of hypertension. Table 11 below lists genes that were identified as consistently and significantly changed in expression levels in subjects with hypertension, following successful relaxation response practice. These genes are suitable for use in assaying for a RR in a subject diagnosed with or at risk for hypertension.

TABLE 11
A preferred list of genes top focus genes identified from
systems biology analysis of genes that are significantly
dysregulated after 8 weeks of RR practice in Hypertensive
patients. Expression of these genes, alone or in combination
with a more complete RR signature (4 networks of 20 Focus
hubs each) can be evaluated.

| | | Directional |
|---|---|---|
| | | change (~1.2 |
| Gene Symbol | Description | fold) |
| IRS1 | Insulin receptor substrate 1 | UP |
| AKT | Protein Kinase B | UP |
| UBC | Ubiquitin C | Down |
| CDC42 | Cell division cycle 42 | UP |
| NFKB | Nuclear Factor-KappaB | Down |
| TP53 | tumor protein p53 | Down |
| MAPK1 | mitogen-activated protein kinase 1 | Down |
| ANGPT2 | Angiopoietin-2 | Down |
| INS | insulin | UP |
| UBC | ubiquitin C | Down |
| MAPK14/ | mitogen-activated protein kinase 14 | Down |
| P38MAPK | | |

### Example 7

### Combined Analysis of Healthy Patient, Hypertensive Patient, and IBD/IBS Patient Relaxation Response Profiles

The Interactive analysis of data from healthy subjects as well as from hypertension and IBD/IBS patients (in Example 5 above) identified NF-κB as the most interactive molecule (Top Focus Hub) regulating the activity of genes involved in blunting inflammation. The activity of the NF-κB complex can be measured by profiling expression of genes such as NF-κB, RELA, MAPK14, MAPK, JNK, and TNF. A list of genes reflective of NF-κB activity is shown below in Table 12.

TABLE 12
Genes reflective of NF-κB activity. The genes were selected
from following analysis i) RR long term and progressive network
enriched with NF-κB upstream and downstream genes generated
from systems biology analysis of temporal data of healthy
subjects after RR Practice, ii) highly interactive focus hub
genes identified from systems biology analysis of genes that are
significantly dysregulated after 8 weeks of RR practice in
Hypertensive or IBD/IBS patients.

| | | Directional |
|---|---|---|
| | | change |
| | | (~1.2 |
| Gene Symbol | Description | fold) |
| RELA/NFKB | v-rel reticuloendotheliosis viral oncogene | Down |
| | homolog A (avian), Nuclear | |
| | Factor-KappaB | |
| TP53 | tumor protein p53 | Down |
| MAPK1 | mitogen-activated protein kinase 1 | Down |
| MAPK14 | mitogen-activated protein kinase 14 | Down |
| JUN | jun oncogene | Down |
| MAP3K1 | mitogen-activated protein kinase kinase | Down |
| | kinase 1 | |
| IKBKG | inhibitor of kappa light polypeptide gene | Down |
| | enhancer in B-cells, kinase gamma | |
| MAPK8 | mitogen-activated protein kinase 8 | Down |
| TRAF6 | TNF receptor-associated factor 6 | Down |
| MYC | v-myc myelocytomatosis viral oncogene | Down |
| | homolog (avian) | |
| TNF | tumor necrosis factor | Down |
| TNFSF11 | tumor necrosis factor (ligand) superfamily, | Down |
| | member 11 | |

In addition, the expression or activity of ATPASE (ATP5C1, ATP5B) and Insulin (Ins) can be measured as these molecules were identified as top focus genes in both healthy subjects and hypertension patients. These molecules depict expression changes after 60 minutes, following listening to a RR CD, as well as after 8 weeks of RR practice, with significant changes in the latter. A list of genes reflective of insulin activity is shown below in Table 13.

TABLE 13
Genes reflective of insulin activity. The genes were identified
by the following analysis i) insulin and energy metabolism
network generated from systems biology analysis of temporal data
of healthy subjects after RR Practice, ii) highly interactive
focus hub genes identified from systems biology analysis of genes
that are significantly dysregulated after 8 weeks of RR in
Hypertensive or IBD/IBS patients.

| | | Directional |
|---|---|---|
| | | change |
| Gene | | (~1.2 |
| Symbol | Description | fold) |
| INS | insulin-like growth factor 2 (somatomedin A); | UP |
| | insulin; INS-IGF2 readthrough transcript | |
| PKM2 | similar to Pyruvate kinase, isozymes M1/M2 | UP |
| | (Pyruvate kinase muscle isozyme) | |
| | (Cytosolic thyroid hormone- | |
| | binding protein) (CTHBP) (THBP1); | |
| | pyruvate kinase, muscle | |
| ATP5C1 | ATP synthase, H+ transporting, mitochondrial | UP |
| | F1 complex, gamma polypeptide 1 | |
| ATP5B | ATP synthase, H+ transporting, mitochondrial | UP |
| | F1 complex, beta polypeptide | |
| CBL | Cas-Br-M (murine) ecotropic retroviral | UP |
| | transforming sequence | |
| UGDH | UDP-glucose dehydrogenase | UP |
| IRS1 | Insulin receptor substrate 1 | UP |

Table 14, below shows a list of prevalently dysregulated genes following RR practice, and the direction of change in expression, identified from the combined analysis of healthy subjects and also hypertension and IBD/IBS patients. Analysis of these genes in the methods discussed herein is expected to produce an indication of the relaxation response in a subject.

Table 15, below, shows a more comprehensive list of 72 genes identified as significantly dysregulated genes following RR practice, and the direction of change in expression, identified from the combined analysis of healthy subjects and also hypertension and IBD/IBS patients. Analysis of these genes in the methods discussed herein, or a subset thereof (e.g., as discussed herein), is expected to produce an indication of the relaxation response in a subject.

TABLE 14
Top most abundant focus gene hubs identified from analysis of
healthy subjects as well as from hypertension and IBD/IBS
patients. Expression of these genes can be evaluated alone or
in combination with other genes discussed herein (e.g. 4
networks of 20 Focus hubs each), to give a relaxation response
signature.

| | | Directional |
|---|---|---|
| | | change |
| Gene | | (~1.2 |
| Symbol | Description | fold) |
| NFKB | Nuclear Factor-KappaB | Down |
| RELA | v-rel reticuloendotheliosis viral oncogene | |
| | homolog A | |
| TP53 | tumor protein p53 | Down |
| MAPK1 | mitogen-activated protein kinase 1 | Down |
| MAPK14 | mitogen-activated protein kinase 14 | Down |
| JUN | jun oncogene | Down |
| MAPK8 | mitogen-activated protein kinase 8 | Down |
| MYC | v-myc myelocytomatosis viral oncogene | Down |
| | homolog (avian) | |
| TNF | tumor necrosis factor | Down |
| INS | insulin | UP |
| ATP5C1 | ATP synthase, H+ transporting, mitochondrial | UP |
| | F1 complex, gamma polypeptide 1 | |
| ATP5B | ATP synthase, H+ transporting, mitochondrial | UP |
| | F1 complex, beta polypeptide | |
| IRS1 | Insulin receptor substrate 1 | UP |

TABLE 15
Focus genes identified using systems biology analysis of Relaxation Response
data from healthy subjects, hypertensive and Inflammatory Bowel Diseases
(IBD)/inflammatory Bowel Syndrome (IBS) patients. The focus genes or their regulatory
targets depict upregulation and downregulation of gene expression/activity in temporal
manner during a session of RR after 8 week or long term of RR practice. This is RR
signature in healthy subjects consisting of 4 networks of 20 Focus hubs each.

| | | |
|---|---|---|
| | | Directional |
| | | change |
| | | (~1.2 |
| Gene Symbol | Description | fold) |

Focus hubs from RR Progressively upregulated pathways

| | | |
|---|---|---|
| PRKCA | protein kinase C, alpha | UP |
| FGFR1 | fibroblast growth factor receptor 1 | UP |
| STX1A | syntaxin 1A (brain) | UP |
| AHCY | adenosylhomocysteinase | UP |
| GNAI2 | guanine nucleotide binding protein (G protein), alpha inhibiting | UP |
| | activity polypeptide 2 | |
| GRB2 | growth factor receptor-bound protein 2 | UP |
| ATP5B | ATP synthase, H+ transporting, mitochondrial F1 complex, beta | UP |
| | polypeptide | |
| CBL | Cas-Br-M (murine) ecotropic retroviral transforming sequence | UP |
| UGDH | UDP-glucose dehydrogenase | UP |
| SRC | v-src sarcoma (Schmidt-Ruppin A-2) viral oncogene homolog | UP |
| | (avian) | |
| POLR2A | polymerase (RNA) II (DNA directed) polypeptide A, 220 kDa | UP |
| GNB2 | guanine nucleotide binding protein (G protein), beta polypeptide 2 | UP |
| RPL7 | ribosomal protein L7 pseudogene 26; ribosomal protein L7 | UP |
| | pseudogene 16; ribosomal protein L7; ribosomal protein L7 | |
| | pseudogene 32; ribosomal protein L7 pseudogene 23; ribosomal | |
| | protein L7 pseudogene 24; ribosomal protein L7 pseudogene 20 | |
| INS | insulin-like growth factor 2 (somatomedin A); insulin; INS-IGF2 | UP |
| | readthrough transcript | |
| PKM2 | similar to Pyruvate kinase, isozymes M1/M2 (Pyruvate kinase | UP |
| | muscle isozyme) (Cytosolic thyroid hormone-binding protein) | |
| | (CTHBP) (THBP1); pyruvate kinase, muscle | |
| ATP5C1 | ATP synthase, H+ transporting, mitochondrial F1 complex, gamma | UP |
| | polypeptide 1 | |
| PRKACA | protein kinase, cAMP-dependent, catalytic, alpha | UP |
| PDHA1 | pyruvate dehydrogenase (lipoamide) alpha 1 | UP |
| SSR4 | signal sequence receptor, delta (translocon-associated protein | UP |
| | delta) | |
| GAPDH | glyceraldehyde-3-phosphate dehydrogenase-like 6; hypothetical | UP |
| | protein LOC100133042; glyceraldehyde-3-phosphate | |
| | dehydrogenase | |

Focus hubs from RR Progressively Downregulated pathways

| | | |
|---|---|---|
| FUS | fusion (involved in t(12;16) in malignant liposarcoma) | Down |
| DHX9 | DEAH (Asp-Glu-Ala-His) box polypeptide 9 | Down |
| YWHAZ | tyrosine 3-monooxygenase/tryptophan 5-monooxygenase | Down |
| | activation protein, zeta polypeptide | |
| TP53 | tumor protein p53 | Down |
| SNRPD2 | small nuclear ribonucleoprotein D2 polypeptide 16.5 kDa; similar to | Down |
| | hCG2040270 | |
| SRC | v-src sarcoma (Schmidt-Ruppin A-2) viral oncogene homolog | Down |
| | (avian) | |
| YBX1 | Y box binding protein 1 | Down |
| PTPN11 | protein tyrosine phosphatase, non-receptor type 11; similar to | Down |
| | protein tyrosine phosphatase, non-receptor type 11 | |
| PLCG1 | phospholipase C, gamma 1 | Down |
| PTK2B | PTK2B protein tyrosine kinase 2 beta | Down |
| MAPK14 | mitogen-activated protein kinase 14 | Down |
| JUN | jun oncogene | Down |
| SOS1 | son of sevenless homolog 1 (Drosophila) | Down |
| SNRPB | small nuclear ribonucleoprotein polypeptides B and B1 | Down |
| JAK1 | Janus kinase 1 | Down |
| HSPA5 | hypothetical gene supported by AF216292; NM_005347; heat | Down |
| | shock 70 kDa protein 5 (glucose-regulated protein, 78 kDa) | |
| TRAF6 | TNF receptor-associated factor 6 | Down |
| MYC | v-myc myelocytomatosis viral oncogene homolog (avian) | Down |
| HSPA8 | heat shock 70 kDa protein 8 | Down |
| NFX1 | nuclear transcription factor, X-box binding 1 | Down |

Focus hubs from Progressive + Long-Term RR Downregulated pathways

| | | |
|---|---|---|
| POLR2H | polymerase (RNA) II (DNA directed) polypeptide H | Down |
| U2AF2 | U2 small nuclear RNA auxiliary factor 2 | Down |
| RELA/NFKB | v-rel reticuloendotheliosis viral oncogene homolog A | Down |
| | (avian), Nuclear Factor-KappaB | |
| TP53 | tumor protein p53 | Down |
| HLA-B | major histocompatibility complex, class I, C; major | Down |
| | histocompatibility complex, class I, B | |
| MAPK1 | mitogen-activated protein kinase 1 | Down |
| FYN | FYN oncogene related to SRC, FGR, YES | Down |
| MAPK14 | mitogen-activated protein kinase 14 | Down |
| JUN | jun oncogene | Down |
| MAP3K1 | mitogen-activated protein kinase kinase kinase 1 | Down |
| LCK | lymphocyte-specific protein tyrosine kinase | Down |
| IKBKG | inhibitor of kappa light polypeptide gene enhancer in B-cells, kinase | Down |
| | gamma | |
| RHOA | ras homolog gene family, member A | Down |
| PIK3CA | phosphoinositide-3-kinase, catalytic, alpha polypeptide | Down |
| MAPK8 | mitogen-activated protein kinase 8 | Down |
| SHC1 | SHC (Src homology 2 domain containing) transforming protein 1 | Down |
| TRAF6 | TNF receptor-associated factor 6 | Down |
| MYC | v-myc myelocytomatosis viral oncogene homolog (avian) | Down |
| PIK3R1 | phosphoinositide-3-kinase, regulatory subunit 1 (alpha) | Down |
| HSPA8 | heat shock 70 kDa protein 8 | Down |

Focus hubs from RR Long Term Upregulated pathways

| | | |
|---|---|---|
| PARD6A | par-6 partitioning defective 6 homolog alpha (C. elegans) | UP |
| HIST1H2BC | histone cluster 1, H2bi; histone cluster 1, H2bg; histone cluster 1, | UP |
| | H2be; histone cluster 1, H2bf; histone cluster 1, H2bc | |
| INADL | InaD-like (Drosophila) | UP |
| PARD3 | par-3 partitioning defective 3 homolog (C. elegans) | UP |
| BRF1 | BRF1 homolog, subunit of RNA polymerase III transcription | UP |
| | initiation factor IIIB (S. cerevisiae) | |
| IL8 | interleukin 8 | UP |
| POLR2K | polymerase (RNA) II (DNA directed) polypeptide K, 7.0 kDa | UP |
| CXCL2 | chemokine (C—X—C motif) ligand 2 | UP |
| CYC1 | cytochrome c-1 | UP |
| GNG12 | guanine nucleotide binding protein (G protein), gamma 12 | UP |
| PLG | plasminogen | UP |
| RPA1 | replication protein A1, 70 kDa | UP |
| GNB2 | guanine nucleotide binding protein (G protein), beta polypeptide 2 | UP |
| GNB1 | guanine nucleotide binding protein (G protein), beta polypeptide 1 | UP |
| F2 | coagulation factor II (thrombin) | UP |
| PCNA | proliferating cell nuclear antigen | UP |
| POU2F1 | POU class 2 homeobox 1 | UP |
| RUVBL1 | RuvB-like 1 (E. coli) | UP |
| H2AFX | H2A histone family, member X | UP |
| CACNA1C | calcium channel, voltage-dependent, L type, alpha 1C subunit | UP |

1.-38. (canceled)

39. A method of generating a relaxation gene expression profile of a subject, comprising:
a) measuring the expression levels of one or more relaxation responsive genes in a biological sample of the subject prior to and subsequent to relaxation practice by the subject;
b) calculating any difference in the measured expression levels of the one or more genes; and
c) recording the respective calculated differences in the measured expression levels of the one or more genes;
to thereby generate the relaxation gene expression profile of the subject.

40. The method of claim 39, wherein the one or more genes are selected from the group consisting of the genes shown in Table 15, or a subset thereof, the genes shown in Table 10, or a subset thereof, the genes shown in Table 11, or a subset thereof, the genes shown in Table 12, or a subset thereof, the genes shown in Table 13, or a subset thereof, the genes shown in Table 14 or a subset thereof, and combinations thereof.

41. The method of claim 39, wherein the one or more genes comprise:
a) NF-κB, RELA, MAPK14, MAPK, JNK, TNF, INS, ATP5C1, and ATP5B;
b) MAPK, JNK, NF-κB, IKBKB, TP53, MYC, and TNF;
c) MAPK, NJK, NF-κB, MAP3K and/or MYC;
d) ATPASE, INS and/or INSR, MAPK14, JUN and/or JNK, NF-κB, MAPK, IKBKG and/or IKBKB, and MAPK; or
e) ATP5C1, ATP5B, INS, PRKACA, MAPK14, JUN, RELA and/or NF-κB, MAPK8, IKBKG, MAP3K1, HSPA5, TP53, CCNA1C, CYC1, and RUVBL.

42. The method of claim 39, wherein the relaxation practice is at least about 20 minutes in duration.

43. The method of claim 39, wherein the biological sample is whole blood.

44. The method of claim 39, wherein the biological sample is peripheral blood mononuclear cells.

45. The method of claim 39, wherein the measuring prior to the relaxation practice is performed in the time period selected from the group consisting of within 2 minutes of relaxation practice by the subject, within 1 minute of completion of the relaxation practice by the subject, within 15 minutes post completion of the relaxation practice by the subject.

46. The method of claim 39, wherein the measuring subsequent to relaxation practice is performed at more than one time point.

47. The method of claim 39, wherein measuring the gene expression levels is performed by quantitative PCR, microarray analysis, luminex gene platform analysis, or next generation sequencing analysis.

48. A method of determining effectiveness of relaxation practice by a subject, comprising:
a) generating a relaxation gene expression profile of the subject in response to relaxation practice by the subject; and
b) comparing the relaxation gene expression profile generated to a standard relaxation gene expression profile indicative of a complete relaxation response;
wherein the similarity of the relaxation gene expression profile generated for the subject to the standard gene expression profile indicates effectiveness of the relaxation practice by the subject.

49. The method of claim 48, wherein the subject is diagnosed with, or at risk for, a disease or disorder caused or exacerbated by stress, selected from the group consisting of hypertension, anxiety, depression, infertility, insomnia, menopausal symptoms, premenstrual symptoms, pains, phobias, nausea, post-traumatic stress disorder, obesity, impotency related to stress or anxiety, tinnitus or sensation of sounds, postoperative swelling, allergic skin reactions, bronchial asthma, congestive heart failure, constipation, cough, diabetes mellitus, drowsiness, duodenal ulcers, fatigue and dizziness, herpes simplex, irritable bowel syndrome and inflammatory bowel disease, rheumatoid arthritis, and multiple sclerosis.

50. The method of claim 48, wherein the relaxation practice is selected from the group consisting of Qigong, yoga, meditation, repetitive prayer, tai chi, breathing exercises, progressive muscle relaxation, biofeedback, hypnosis, autogenic training and guided imagery.

51. A method for treating a subject diagnosed with, or at risk for a disease or disorder, comprising:
a) assaying for the relaxation response in the subject by the method of claim 48; and
b) prescribing relaxation response therapy, in lieu of, or in combination with, drug therapy appropriate for treatment of the disease or disorder, for the subject if identified as exhibiting the relaxation response, or prescribing drug therapy appropriate for treatment of the disease or disorder for the subject if identified as not exhibiting the relaxation response.

52. The method of claim 51, wherein the disease or disorder is caused or exacerbated by stress and is selected from the group consisting of hypertension, anxiety, depression, infertility, insomnia, menopausal symptoms, premenstrual symptoms, pains, phobias, nausea, post-traumatic stress disorder, obesity, impotency related to stress or anxiety, tinnitus or sensation of sounds, postoperative swelling, allergic skin reactions, bronchial asthma, congestive heart failure, constipation, cough, diabetes mellitus, drowsiness, duodenal ulcers, fatigue and dizziness, herpes simplex, irritable bowel syndrome and inflammatory bowel disease, rheumatoid arthritis, and multiple sclerosis.

53. A microarray for high throughput detection of gene expression levels in a biological sample comprising probes for one or more relaxation-responsive genes.

54. The microarray of claim 53, wherein the one or more relaxation-responsive genes are selected from the group consisting of the genes shown in Table 15, or a subset thereof, the genes shown in Table 10, or a subset thereof, the genes shown in Table 11, or a subset thereof, the genes shown in Table 12, or a subset thereof, the genes shown in Table 13, or a subset thereof, the genes shown in Table 14 or a subset thereof, and combinations thereof.

55. The microarray of claim 54, wherein the one or more genes comprise:
a) NF-κB, RELA, MAPK14, MAPK, JNK, TNF, INS, ATP5C1, and ATP5B;
b) MAPK, JNK, NF-κB, IKBKB, TP53, MYC, and TNF;
c) MAPK, NJK, NF-κB, MAP3K and/or MYC;
d) ATPASE, INS and/or INSR, MAPK14, JUN and/or JNK, NF-κB, MAPK, IKBKG and/or IKBKB, and MAPK; or
e) ATP5C1, ATP5B, INS, PRKACA, MAPK14, JUN, RELA and/or NF-κB, MAPK8, IKBKG, MAP3K1, HSPA5, TP53, CCNA1C, CYC1, and RUVBL.
